(19) United States
(12) Patent Application Publication
(10) Pub. No.: US 2011/0172295 A1
(43) Pub. Date: Jul. 14, 2011

(54) METHODS AND COMPOSITIONS FOR THE REGULATION OF microRNA PROCESSING

(75) Inventors: Scott M. Hammond, Pittsboro, NC (US); Martin A. Newman, Chapel Hill, NC (US); J. Michael Thomson, Nashville, TN (US)

(73) Assignee: THE UNIVERSITY OF NORTH CAROLINA AT CHAPEL HILL, Chapel Hill, NC (US)

(21) Appl. No.: 12/937,194

(22) PCT Filed: Apr. 6, 2009
(86) PCT No.: PCT/US09/39629

§ 371 Date: Dec. 20, 2010

Related U.S. Application Data
(60) Provisional application No. 61/123,988, filed on Apr. 11, 2008.

(51) Int. Cl. A61K 31/7088; C12N 5/10; C12N 5/071; C12N 1/00; C12N 15/63; C07H 21/02; A61P 35/00; A61K 48/00

(52) U.S. Cl. 514/44 R; 435/325; 435/375; 435/377; 435/419; 435/243; 435/320.1; 536/24.1

(57) ABSTRACT

Provided herein are methods and compositions for the regulation of microRNA processing. The invention presents methods for inhibiting the activity of a Lin-28-like polypeptide and methods for treating a subject with cancer comprising administering to a subject with cancer a compound that inhibits the activity of a Lin-28-like polypeptide. Compositions comprise isolated polynucleotides and expression cassettes comprising the same that promote the processing of at least one of a regulated primary microRNA transcript by Drosha and a regulated precursor microRNA by Dicer when introduced into or expressed in a cell. Compositions and methods are also provided for the selective suppression of target polynucleotides by a microRNA based on the level of expression of a Lin-28-like polypeptide within a cell.

### FEDERALLY SPONSORED RESEARCH OR DEVELOPMENT

This invention was made with United States Government support under grant number GM070674 awarded by the National Institutes of Health. The United States government has certain rights in the invention.

### FIELD OF THE INVENTION

The present invention relates to the field of molecular biology and the regulation of gene expression, in particular, the regulation of microRNA processing.

### BACKGROUND OF THE INVENTION

Diverse forms of double stranded RNA can act as triggers of RNA interference (RNAi) or related homology dependent gene silencing pathways (Chapman & Carrington (2007) Nat Rev Genet. 8:884-896). Among these triggers are microRNAs (miRNAs), noncoding polynucleotides of about 19 to about 25 nucleotides in length that are processed from primary transcripts encoded by endogenous genes. miRNAs are encoded in the genomes of most metazoans and function in a post-transcriptional layer of gene regulation through miRNA-directed degradation or translational inhibition of target mRNAs (see Bartel (2004) Cell 116:281-297 for a review).

The founding miRNA, lin-4, was discovered in C. elegans as a mutant that displayed heterochronic, or developmental timing defects (Lee, Feinbaum & Ambros (1993) Cell 75:843-854; Wightman, Ha & Ruvkun (1993) Cell 75:855-862). The activity of this small RNA is mediated largely through repression of two well established target mRNAs, lin-14 and lin-28 (Ambros (1989) Cell 57:49-57). A second miRNA, let-7, was later identified as a heterochronic mutant (Reinhart et al. (2000) Nature 403:901-906). Surprisingly, this miRNA has complete nucleotide conservation from C. elegans to humans, suggesting an ancient biological role (Pasquinelli et al. (2000) Nature 408:86-89). More recently, thousands of miRNAs have been identified across many phyla. Computational predictions suggest that each miRNA has tens to hundreds of targets, underscoring their immense potential for controlling gene expression (Bartel (2004) Cell 116:281-297).

### BRIEF SUMMARY OF THE INVENTION

The present invention provides isolated polynucleotides and expression cassettes comprising the same that promote the processing of at least one of regulated primary microRNA transcripts by Drosha and regulated precursor microRNAs by Dicer when introduced into or expressed in a cell. These isolated polynucleotides can be introduced into a cell as a method for differentiating a cell or inhibiting the growth of a cell. Further provided herein are methods for inhibiting the activity of a Lin-28-like polypeptide comprising contacting a cell with a compound that reduces the Lin-28-like polypeptide-mediated inhibition of at least one of regulated primary microRNA transcript processing by Drosha and regulated precursor microRNA processing by Dicer. The presently disclosed subject matter also provides methods for treating a cancer comprising administering to a subject in need thereof an effective amount of a compound that inhibits the activity of a Lin-28-like polypeptide. In addition, isolated polynucleotides comprising or encoding a stem-loop structure are provided herein, wherein the loop sequence is from a regulated primary microRNA transcript or a regulated precursor microRNA or an active variant or fragment thereof, and wherein the stem of the stem-loop structure comprises a miRNA. Such polynucleotides are useful for the treatment of diseases such as cancer and for the selective suppression of target polynucleotides in a population of cells, which in some embodiments can lead to the enrichment of a subpopulation of cells, such as stem cells.

### BRIEF DESCRIPTION OF THE FIGURES

FIG. 1A shows an autoradiograph, the quantitation of which is depicted in FIG. 1B, demonstrating that embryonic cells contain a Drosha inhibitor that regulates Let-7 processing. Radiolabeled primary miRNA (pri-miRNA) substrates corresponding to Let-7g and miR-17 were incubated in P19 or Hela nuclear extracts, as indicated. Drosha protein, or mock, was immunoprecipitated with a polyclonal antibody Immobilized protein was incubated with Let-7g and miR-17 primar miRNA (pri-miRNA) substrates, as indicated. Drosha endonuclease products were resolved on a denaturing polyacrylamide gel. The production of Let-7g and miR-17 precursors is indicated by arrows. A labeled RNA oligonucleotide ladder is shown for size reference. In FIG. 1B, the ratio of Let-7g product to miR-17 product was quantitated using ImageQuant software and is plotted as a bar graph.

FIG. 2 presents a sequence alignment of Let-7 family members. The mature miRNA sequence, complementary stem strand (not exactly the star strand), and loop region are indicated. Gray boxes indicate regions of homology. Arrows indicate nucleotides that were mutagenized. Sequence changes are indicated below the alignment. mmu: Mus musculus; gga: Gallus gallus; bta: Bos taurus; xtr: Xenopus tropicalis; cel: Caenorhabditis elegans.

FIG. 3 provides an autoradiograph indicating Drosha processing products. Wild type or mutant pri-Let-7g substrates were combined with the pri-miR-17 substrate and incubated in a P19 nuclear extract. Drosha products were resolved and are indicated.

FIG. 4 provides an autoradiograph indicating Drosha processing products. Pri-miRNA substrates for Let-7g and miR-17 were combined and incubated in P19 nuclear extracts. Competitor RNA transcripts corresponding to the loop plus 12 nucleotides of each stem, or competitor 2′-O methyl oligonucleotides, were included at 10, 50, and 250 nM final concentration. In one case, the oligonucleotide had a 3′ biotin moiety. The left lane had no competitor. Drosha products were resolved on a denaturing polyacrylamide gel. Precursor products are indicated by arrows.

FIG. 5 shows a Coomassie blue-stained polyacrylamide gel that has resolved proteins that have been captured with a capture probe comprising the Let-7d loop region. Oligonucleotide capture probes corresponding to the Let-7d loop or a random (control) sequence, fully 2′-O-methyl modified and 3′ biotin linked, were bound to streptavidin agarose. Proteins were captured from P19 nuclear extracts, were resolved on a 4-20% polyacrylamide gel, and stained with Coomassie blue. Proteins were isolated and identified by MALDI-TOF fingerprinting.

FIG. 6 presents an autoradiograph, wherein increasing concentrations of a cold Let-7g competitor oligonucleotide inhibited the interaction of Let-7d with Lin-28. Radiolabeled RNA probes corresponding to the Let-7d stem-loop or miR-20a stem-loop were incubated with P19 nuclear extracts. Non-labeled Let-7d loop oligonucleotide competitors were included as indicated. Proteins were crosslinked to probes with UV light. Lin-28 was immunoprecipitated from crosslink reactions with a polyclonal antibody. Total extract (IP input) and immunoprecipitates were resolved, as indicated, on a polyacrylamide gel.

FIG. 7 presents a heatmap from a microarray study, along with hierarchical clustering of microRNAs whose expression was reduced upon ectopic Lin-28 or Lin-28B expression. NIH-3T3 cells were transduced with MSCV retroviral constructs that drive expression of Lin-28 or Lin-28B or control, as indicated. Steady state miRNA expression levels were quantitated using a custom microarray platform 10 days post-infection. Normalized measurements were hierarchically clustered and are plotted as a heat map. Light gray indicates high expression and dark gray low expression, relative to the mean. Let-7 family members are underlined.

FIG. 8 provides steady state miRNA expression levels from NIH-3T3 cells (from FIG. 7) expressing Lin-28, Lin-28B, or control. The miRNA expression levels were quantitated by real time RT-PCR. U6 snRNA was used as the reference. Expression of pri-Let-7g was also quantitated by real time RT-PCR. β2-microglobulin was used as the reference.

FIG. 9 presents steady state miRNA expression levels from P19 cells transfected with siRNAs targeting Lin-28. Two effective siRNAs were used alone or in combination. Five days post-transfection, mature miRNA levels were measured by real time RT-PCR. U6 was used as a reference. Expression of pri-Let-7g was also quantitated by real time RT-PCR. β2-microglobulin was used as the reference.

FIG. 10 presents an autoradiograph demonstrating that purified recombinant Lin-28 inhibits Let-7g processing. Pri-miRNA substrates for Let-7g and miR-17 were combined and incubated in Hela nuclear extracts. Purified, recombinant NF-45 (control) or Lin-28 were included at 1, 10, and 100 ng/μl final concentration. Drosha products were resolved on a denaturing polyacrylamide gel. Precursor products are indicated by arrows. Recombinant protein was produced in E. coli.

FIG. 11 presents an autoradiograph demonstrating that purified recombinant Lin-28 inhibits Let-7g processing when endogenous Lin-28 has been immunodepleted from extracts. Polyclonal Lin-28 antibody, or mock, was bound to protein A sepharose. P19 nuclear extracts were incubated with immobilized antibody. Resultant immunodepleted extracts were incubated with pri-miRNA substrates for Let-7g and miR-17. In right lanes, recombinant Lin-28 was added back to immunodepleted reactions at 100 ng/μl final concentration. Drosha products were resolved on a denaturing polyacrylamide gel. Precursor products are indicated by arrows. Recombinant protein was produced in HEK-293 cells.

FIG. 12 presents data depicting Drosha processing of Let-7g and miR-17 in differentiated P19 extracts. P19 cells were differentiated in retinoic acid for 10 days. Nuclear extracts were made from differentiated and undifferentitiated P19 cells. Radiolabeled pri-miRNA substrates corresponding to Let-7g and miR-17 were incubated in nuclear extracts, as indicated. Drosha products were resolved on a denaturing polyacrylamide gel. The production of Let-7g and miR-17 precursors is indicated by arrows.

FIG. 13 provides verification of Drosha products. Hela nuclear extracts were incubated with labeled pri-Let-7g and pri-miR-17 (left panel) or unlabeled pri-Let-7g and pri-miR-17 (center and right panels, respectively). Drosha products were resolved on a denaturing polyacrylamide gel. Reaction products were visualized by direct autoradiography (left panel) or by northern blot analysis (center and right panel). Northern blot probes consisted of locked nucleic acid oligonucleotides complementary to the mature region of the indicated miRNAs.

FIG. 14 presents an autoradiograph demonstrating that the SD2 mutant partially releases the Drosha block. Wild type or mutant SD2 pri-Let-7g substrates were combined with the pri-miR-17 substrate and incubated in a P19 or Hela nuclear extract, as indicated. Drosha products were resolved and are indicated. The ratio of Let-7g product to miR-17 product was quantitated using ImageQuant software. SD2 led to a 2.2 fold increase in relative Let-7g processing.

FIG. 15 depicts a crosslinking analysis using non-specific control competitors. Radiolabeled RNA probes corresponding to the Let-7d stem-loop were incubated with P19 nuclear extracts. Non-labeled Let-7d loop oligonucleotide competitors, or non-specific oligonucleotide competitors were included as indicated. Proteins were crosslinked to probes with UV light. Lin-28 was immunoprecipitated from crosslink reactions with a polyclonal antibody. Immunoprecipitates were resolved on a polyacrylamide gel.

FIG. 16 provides a Coomassie blue-stained polyacrylamide gel demonstrating purified NF-45 and Lin-28 recombinant proteins. NF-45 and Lin-28 were expressed in E. coli and purified by metal ion affinity chromatography. Proteins were resolved on a polyacrylamide gel and stained with Coomassie blue. Lin-28 appears as multiple bands for unknown reasons. This is also observed with endogenous protein in mouse embryonic cells.

FIG. 17 depicts an immunoblot demonstrating the level of Lin-28 and actin (control) proteins in the presence of GAPDH (control) or Lin-28-targeted siRNAs. Multiple siRNAs against Lin-28 were designed and transfected into P19 cells alone, or in combination. Knockdown is represented by western blotting with a polyclonal Lin-28 antibody.

FIG. 18 shows the expression level of ectopically expressed Lin-28 protein. Lin-28 was ectopically expressed in NIH-3T3 cells by retroviral transduction. Expression level was quantitated by western blotting using a polyclonal Lin-28 antibody. P19 whole cell extract is shown for reference.

FIG. 19 shows an autoradiograph demonstrating that Lin-28 blocks Dicer-mediated processing of precursor miRNAs. RNA oligonucleotides comprising a PLK-1 miRNA sequence and a Let-7a2 loop sequence are not efficiently converted to miRNA duplexes in the presence of recombinant Lin-28 (left gel). A control RNA oligonucleotide comprising the identical PLK-1 miRNA but with an artificial loop sequence is converted to a miRNA duplex in the presence of identical concentrations of Lin-28 (right gel).

FIG. 20 provides an alignment between the microRNA loop sequences of Homo sapiens let-7a-3 (SEQ ID NO: 5), let-7f-1 (SEQ ID NO: 3), let-7b (SEQ ID NO: 11), let-7f-2 (SEQ ID NO: 7), let-7a-1 (SEQ ID NO: 9), let-7a-2 (SEQ ID NO: 2), let-7c (SEQ ID NO: 4), let-7e (SEQ ID NO: 10), let-7d (SEQ ID NO: 1), let-71 (SEQ ID NO: 6), and let-7g (SEQ ID NO: 8); Mus musculus let-7g (SEQ ID NO: 8) and let-71 (SEQ ID NO: 6); Gallus gallus let-7g (SEQ ID NO: 8) and let-71 (SEQ ID NO: 6), and Xenopus tropicalis let-71 (SEQ ID NO: 6). Also provided is the consensus sequence of these sequences, which is set forth in SEQ ID NO: 102. hsa: Homo sapiens; mmu: Mus musculus; gga: Gallus gallus; xtr: Xenopus tropicalis.

### DETAILED DESCRIPTION OF THE INVENTION

### I. General Overview

The presently disclosed subject matter relates to microRNA biogenesis. Specifically, the methods and compositions provided herein are useful in promoting the biogenesis of particular microRNAs whose processing into mature microRNAs is regulated at a certain step within the biogenesis pathway or to selectively suppress a target polynucleotide with a polynucleotide comprising or encoding a stem-loop structure having a microRNA and a loop sequence that is recognized by an inhibitor of microRNA processing.

As used herein, the terms “microRNA,” “miRNA,” “mature microRNA,” and “mature miRNA” refer to a non-coding single-stranded RNA molecule that is about 19 to about 25 nucleotides in length (including about 19, about 20, about 21, about 22, about 23, about 24, and about 25 nucleotides) that effectively reduces the expression level of target polynucleotides and polypeptides encoded thereby through the RNA interference pathway (i.e., through association with the RISC and subsequent degradation of target mRNA or translational inhibition). The term “microRNA” refers to both endogenous miRNAs that have been found in any organism (e.g., plants, animals) and artificial miRNAs that include single-stranded RNA molecules with sequences of about 19-25 nucleotides in length other than those found in endogenous miRNAs, that effectively reduce the expression of target polynucleotides through RNA interference. While artificial miRNAs have a sequence that is different from endogenous miRNAs, artificial miRNAs may exhibit sequence similarity to an endogenous miRNA and effectively mimic the endogenous miRNA. In some embodiments, the artificial miRNA has at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or greater sequence similarity to an endogenous miRNA. A microRNA may be generated from various precursors, including but not limited to, an endogenous primary microRNA transcript (pri-miRNA), a hairpin RNA comprising a miRNA that has been introduced into a cell (including shRNA molecules), or a transcript comprising a microRNA that has been encoded by plasmid DNA that has been introduced into a cell.

An entity such as a gene or an expression product thereof, is considered “endogenous” to a cell if it is naturally present within the cell in the absence of modification of the cell, or an ancestor of the cell, by the hand of man. It will be appreciated that the amount of an endogenous miRNA present within a cell can be increased above its naturally occurring level by introducing a polynucleotide comprising or encoding an endogenous miRNA sequence.

A. MicroRNA Biogenesis

Endogenous miRNAs are generated through a series of steps beginning with the transcription of a primary miRNA transcript (pri-miRNA) that is encoded by a microRNA gene. MicroRNA genes can be found throughout the genome, including within coding regions for polypeptides (see Kim (2005) Nat Rev Mol Cell Biol 6:376-385 for review on microRNA biogenesis). The pri-miRNA transcript is a single-stranded RNA molecule that can be recognized and cleaved by Drosha and that comprises at least one stem-loop structure with a microRNA sequence incorporated therein. Endogenous pri-miRNA transcripts are typically thousands of nucleotides long and are often capped, spliced, and poly-adenylated. Under some circumstances, the primary microRNA transcript can be found within the introns of a messenger RNA (mRNA) that encodes a polypeptide. In these instances, the primary microRNA transcript is spliced out of the mRNA through splicing mechanisms prior to processing of the resultant primary microRNA transcript by Drosha (as described herein below). The pri-miRNA transcript can be polycistronic, comprising multiple microRNAs of the same or different sequence.

A “stem-loop structure” refers to a polynucleotide having a secondary structure that includes a region of nucleotides which are known or predicted to form a double stranded portion (the stem portion or stem region) that is linked on one side by a region of predominantly single-stranded nucleotides (loop portion or loop region). The term “hairpin” structure is also used herein to refer to a stem-loop structure. The stem-loops of pri-miRNA and precursor miRNAs (pre-miRNAs) comprise the microRNA sequence, which is complementary (fully or partially) to the target mRNA, and base pairs with the microRNA* sequence (also referred to as the “star strand”). The microRNA and microRNA* sequences make up the stem of the stem-loop structure and can be fully or partially complementary to one another.

In addition to endogenous microRNA genes, primary microRNA transcripts can also be encoded by DNA (e.g., an expression cassette) that has been introduced into a cell and that comprises a sequence that encodes the primary microRNA transcript operably linked to regulatory sequences that control the expression of the transcript.

Alternatively, primary microRNA transcripts can also refer to hairpin-comprising single-stranded RNAs that have been introduced into a cell, that comprise a microRNA sequence, and can be recognized and cleaved by the enzyme Drosha to generate a precursor miRNA.

The primary microRNA transcript is processed by the RNase III enzyme Drosha, wherein the pri-miRNA is cleaved by Drosha to release the stem-loop structure comprising the microRNA, which is now referred to as the precursor miRNA or pre-miRNA. Thus, the terms “precursor microRNA,” “pre-miRNA,” and “precursor miRNA” refer to a nucleic acid that can be recognized and processed by Dicer and have a stem-loop structure with a microRNA sequence incorporated therein. Drosha asymmetrically cleaves the primary microRNA transcript at sites near the base of the primary stem loop, creating precursor miRNAs comprising a 5′ phosphate and a short (about 2 nucleotide) 3′ overhang. Precursor miRNAs can have a length of about 50 to about 90 nucleotides, including but not limited to about 50, about 55, about 60, about 65, about 70, about 75, about 80, about 85 nucleotides, or about 90 nucleotides.

Drosha is a protein of approximately 160 kDa in size with two tandem RNase III domains (RIIIDs) and a double-stranded RNA-binding domain (dsRBD) that are all necessary for catalysis. The region of the protein adajacent to the RIIIDs is also essential for pri-miRNA processing. Drosha is found in a large complex (approximately 500-650 kDa) called the Microprocessor complex, which in humans, includes the DiGeorge syndrome critical region gene 8 (DGCR8) protein (also known as Pasha in D. melanogaster and C. elegans). DGCR8/Pasha is an approximately 120 kDa polypeptide containing two dsRBDs and a putative WW domain, which typically interacts with proline-rich sequences. The exact contribution of DGCR8/Pasha to pri-miRNA processing is unclear, but it is believed to be involved in substrate recognition.

Pre-miRNAs are exported from the nuclear compartment into the cytoplasm by exportin-5, where the pre-miRNAs are further processed into miRNA duplexes by the cytoplasmic RNase III Dicer. Dicer is a protein of about 200 kDa, comprising two RIIIDs, a dsRBD, and an N-terminal segment comprising a DEAD-box RNA helicase domain, a DUF283 domain, and a PAZ domain. PAZ domains bind to the 3′ overhanging end of small RNAs, such as the short (about 2 nucleotide) 3′ overhang on pre-miRNAs created by Drosha cleavage of the pri-miRNA transcript. Unlike Drosha, which is restricted to animals, Dicer is found in almost all eukaryotic organisms, including yeast, plants, and animals.

Exogenously introduced small hairpin RNAs (shRNAs) can essentially mimic endogenous precursor miRNAs by comprising a stem-loop structure with a double-stranded stem comprising a miRNA, wherein the shRNA can be recognized by Dicer. As described above, recognition by Dicer occurs through the presence of 3′ overhanging ends (with approximately two nucleotides) on the shRNA molecule. The loop of the shRNA is cleaved by Dicer and the resulting miRNA duplex enters the RISC complex. mRNA duplexes produced from exogenously introduced shRNA molecules can also be referred to herein as small interfering RNA (siRNA).

The miRNA duplexes (double-stranded RNA) generated by Dicer comprise the mature microRNA, which is the strand that will bind to the complementary (fully or partially) target mRNA, and the microRNA* strand or “star strand,” which is complementary to the microRNA itself (fully or partially). The mature miRNA is incorporated into RNA-induced silencing complexes (RISC) and guides the RISC to complementary RNA molecules, wherein the RISC either nucleolytically degrades the target mRNA or blocks the translation of the target mRNA, thereby inhibiting the expression of the target polynucleotide. The strand that is chosen by RISC to direct the degradation or translational inhibition of a complementary target mRNA is dependent upon the degree of stability of the termini of the miRNA duplex. The strand with lower stability base pairing of the 2-4 nucleotides at the 5′ end of the duplex preferentially associates with RISC and thus, becomes the mature miRNA (Schwarz et al. (2003) Cell 115(2): 199-208.

B. Biological Effects of MicroRNAs

MicroRNAs direct the degradation or inhibit the translation of target messenger RNAs (mRNAs), effectively reducing the expression of target polynucleotides.

By “target polynucleotide” is intended a polynucleotide the expression of which is reduced by a microRNA that is complementary (fully or partially) to the target mRNA that is encoded by the target polynucleotide. In some cases, the target mRNA can comprise more than one region that is complementary to a particular microRNA. Likewise, a target polypeptide is the polypeptide encoded by the polynucleotide whose expression is reduced by a miRNA. Thousands of endogenous miRNAs have been identified across many phyla. While few validated mRNA targets have been assigned to these miRNAs, computational predictions suggest that each miRNA has tens to hundreds of target polynucleotides (Bartel (2004) Cell 116:281-297).

The term “expression” has its meaning as understood in the art and refers to the process of converting genetic information encoded in a DNA sequence (coding sequence) into RNA (e.g., mRNA, rRNA, tRNA, or snRNA) through “transcription” of a polynucleotide (e.g., via the enzymatic action of an RNA polymerase), and for polypeptide-encoding polynucleotides, into a polypeptide through “translation” of mRNA. Thus, an “expression product” is, in general, an RNA transcribed from the coding sequence (e.g., either pre- or post-processing) or a polypeptide encoded by an RNA transcribed from the DNA coding sequence (e.g., either pre- or post-modification).

A miRNA reduces or suppresses the expression of target polynucleotides or polypeptides encoded thereby. By “reduces,” “reducing,” “suppresses,” or “suppressing” the expression level of a polynucleotide or a polypeptide encoded thereby is intended to mean the level of the target mRNA that is encoded by the target polynucleotide or the encoded polypeptide is statistically lower than the target mRNA level or encoded polypeptide level in an appropriate control which is not exposed to the miRNA. In particular embodiments of the present invention, reducing or suppressing the target mRNA level and/or the encoded polypeptide level according to the presently disclosed subject matter results in less than 95%, less than 90%, less than 80%, less than 70%, less than 60%, less than 50%, less than 40%, less than 30%, less than 20%, less than 10%, or less than 5% of the target mRNA level, or the level of the polypeptide encoded thereby in an appropriate control. Methods to assay for the level of the target mRNA, the level of the encoded polypeptide, or the activity of the polynucleotide or polypeptide are well known in the art and can include various biochemical techniques, including but not limited to, RNA solution hybridization, nuclease protection, Northern hybridization, reverse transcription-polymerase chain reaction (RT-PCR), microarray, antibody binding, enzyme linked immunosorbent assay (ELISA), Western blotting, radioimmunoassay (RIA), other immunoassays, and fluorescence activated cell analysis (FACS). Also, the consequences of target polynucleotide suppression can be confirmed by examination of the phenotype of the cell or organism.

The target mRNA is nucleolytically degraded or translation of the target mRNA into the target polypeptide encoded thereby is inhibited by microRNA-directed RISC activity. The choice of mRNA degradation or translational inhibition is dictated somewhat by the complementarity between the microRNA and the complementary region of the target mRNA. For those target mRNAs that comprise region(s) that are fully complementary to a microRNA, the target mRNA will be degraded by the microRNA-directed RISC. On the other hand, for those target mRNAs that comprise region(s) that are only partially complementary to a microRNA, the translation of the target mRNA will be inhibited by RISC. In animals, translational inhibition of target mRNAs is the preferred mechanism of post-transcriptional regulation by endogenous microRNAs, whereas most microRNA-targeted mRNAs are degraded in plants.

The term “complementary” is used herein in accordance with its art-accepted meaning to refer to the capacity for pairing via hydrogen bonds (e.g., Watson-Crick base pairing or Hoogsteen base pairing) between two nucleosides, nucleotides or nucleic acids, and the like. For example, if a nucleotide at a certain position of a first nucleic acid is capable of stably hydrogen bonding with a nucleotide located opposite to that nucleotide in a second nucleic acid, when the nucleic acids are aligned in opposite 5′ to 3′ orientation (i.e., in anti-parallel orientation), then the nucleic acids are considered to be complementary at that position (where position may be defined relative to either end of either nucleic acid, generally with respect to a 5′ end). The nucleotides located opposite one another can be referred to as a “base pair.” A complementary base pair contains two complementary nucleotides, e.g., A and U, A and T, G and C, and the like, whereas a noncomplementary base pair contains two noncomplementary nucleotides (also referred to as a mismatch). Two polynucleotides are said to be complementary to each other when a sufficient number of corresponding positions in each molecule are occupied by nucleotides that hydrogen bond with each other, i.e., a sufficient number of base pairs are complementary.

MicroRNAs can be fully or partially complementary to target mRNAs. In some embodiments of the presently disclosed subject matter, microRNAs exhibit at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or higher complementarity to a target mRNA to effect target mRNA degradation or translational inhibition.

The microRNA can be complementary to any region of the target mRNA to effect degradation or translational inhibition. In animals, endogenous microRNAs are typically complementary to the 3′ untranslated region (3′ UTR) of target mRNAs. Although the microRNA can be only partially complementary with the target mRNA, in general, the first 2-8 nucleotides (counting from the 5′ end) of the miRNA is fully complementary with the target mRNA. In some instances, the region of precise sequence complementarity is interrupted by a bulge. See, Ruvkun (2001) Science 294: 797-799, Zeng et al. (2002) Molecular Cell 9:1-20, and Mourelatos et al. (2002) Genes Dev 16:720-728.

There is also evidence that in plants, and possibly in animals, in addition to post-transcriptional silencing mechanisms, miRNAs also might direct transcriptional silencing of the genes that encode miRNA-targeted mRNAs through methylation of the gene. In some circumstances, the polynucleotide encoding the target mRNA (target polynucleotide) is transcriptionally silenced by microRNA-directed chromatin methylation of the target polynucleotide (Bao et al. (2004) Dev. Cell 7:653-662; Mette et al. (2000) EMBO J 19:5194-5201; Hamilton et al. (2002) EMBO J 21:4671-4679; Zilberman et al. (2003) Science 299:716-719).

Data presented herein demonstrate the inhibition of Drosha-mediated processing of certain primary microRNA transcripts into precursor microRNAs by Lin-28-like polypeptides and the effective reversal of this inhibition through the introduction of polynucleotide sequences comprising a nucleotide sequence corresponding to the loop region of these regulated microRNAs into a cell. As is also demonstrated herein, Lin-28 can also inhibit Dicer-mediated processing of precursor-miRNAs into miRNA duplexes. Thus, the presently disclosed subject matter provides methods and compositions for promoting the processing of at least one of regulated primary microRNA transcripts and regulated precursor-miRNAs, methods for differentiating a cell or inhibiting its growth, and for the treatment of cancer with compounds that inhibit the activity of a Lin-28-like polypeptide. Also provided herein are methods for the selective suppression of target polynucleotides and the treatment of diseases such as cancer with stem-loop structures comprising a microRNA and a loop sequence from Lin-28-like polypeptide-regulated primary microRNA transcripts or Lin-28-like polypeptide-regulated precursor-microRNAs.

### II. Compositions

The presently disclosed subject matter provides compounds that promote the processing of at least one of a regulated primary microRNA transcript by Drosha and a regulated precursor microRNA by Dicer when introduced into a cell. As used herein, a “compound” can refer to a small molecule, a polynucleotide, or other naturally occurring or synthetically derived molecule. In some embodiments, the compounds are isolated polynucleotides that exhibit this activity or expression cassettes encoding polynucleotides with the activity. Also provided are host cells and pharmaceutical compositions comprising the isolated polynucleotides and expression cassettes. In some embodiments, such polynucleotides comprise loop regions from regulated primary microRNA transcripts, the Drosha-mediated processing of which is regulated by Lin-28-like polypeptides or loop regions from regulated precursor microRNAs, the Dicer-mediated processing of which is regulated by Lin-28-like polypeptides.

The term “regulated primary microRNA transcript” refers to a primary microRNA transcript that is capable of being processed by Drosha, but under certain physiological or pathological conditions, the processing or cleavage of the primary microRNA transcript by Drosha is inhibited or reduced. In some embodiments, the inhibition of the Drosha-mediated processing is due to the activity of a Lin-28-like polypeptide.

Similarly, the term “regulated precursor microRNA” or “regulated pre-miRNA” refers to a precursor microRNA that is capable of being processed by Dicer, but under certain physiological or pathological conditions, the processing or cleavage of the precursor microRNA by Dicer is inhibited or reduced. In some embodiments, the inhibition of the Dicer-mediated processing is due to the activity of a Lin-28-like polypeptide.

As used herein, when referring to a loop region or loop sequence of a primary microRNA transcript or a precursor microRNA, the loop region is the single-stranded loop of the stem-loop structure of the molecule, with the miRNA sequence comprised in the stem.

As used herein, the term “promote” in reference to Drosha or Dicer processing refers to an increase in or enhancement of Drosha or Dicer processing within a cell when compared to an appropriate control cell. The control cell can be the cell prior to the introduction of a compound that enhances Drosha or Dicer activity or a cell of the same type (e.g., same tissue of origin, developmental stage, phenotype) that does not comprise the compound. In those embodiments wherein the compound comprises a polynucleotide, the control cell is a cell of the same type that does not express the polynucleotide or that expresses the polynucleotide to a lesser degree. The increase in Drosha processing of the regulated primary microRNA transcripts can be due to a reversal of an inhibitory block in Drosha processing (e.g., due to the activity of a Lin-28-like polypeptide). Likewise, the increase in Dicer processing of the regulated precursor microRNAs can be due to the reversal of an inhibitory block in Dicer processing (e.g., due to the activity of a Lin-28-like polypeptide). In some embodiments, the promotion of Drosha processing can be an increase in the processing of a regulated primary microRNA transcript into a precursor miRNA, wherein the increase in activity is at least 5%, at least 10%, at least 20%, at least 30%, at least 40%, at least 50%, at least 60%, at least 70%, at least 80%, at least 90%, at least 95%, at least 96%, at least 97%, at least 98%, at least 99%, or higher. In certain embodiments, the promotion of Dicer processing can be an increase in the processing of a regulated precursor microRNA into a microRNA duplex, wherein the increase in activity is at least 5%, at least 10%, at least 20%, at least 30%, at least 40%, at least 50%, at least 60%, at least 70%, at least 80%, at least 90%, at least 95%, at least 96%, at least 97%, at least 98%, at least 99%, or higher. Drosha and Dicer activity can be measured using assays known in the art, including but not limited to, those described elsewhere herein.

In certain embodiments, the presently disclosed compounds promote at least one of Drosha processing of regulated primary microRNA transcripts and Dicer processing of regulated precursor microRNAs by inhibiting the activity of a Lin-28-like polypeptide, wherein the activity comprises the inhibition of the processing of at least one of a regulated primary microRNA transcript by Drosha and a regulated precursor microRNA by Dicer. In some embodiments, the compounds promote the processing of Drosha-mediated processing of regulated primary microRNA transcripts. In other embodiments, the compounds promote the processing of Dicer-mediated processing of regulated precursor microRNAs. In yet other embodiments, the compounds promote both the processing of Drosha-mediated processing of regulated primary microRNA transcripts and Dicer-mediated processing of regulated precursor microRNAs.

As used herein, the term “activity” in reference to a polypeptide refers to a biological function associated with the polypeptide. An agent that inhibits the activity of a polypeptide does so regardless of the agent's effects on the expression level of the protein and an agent that solely reduces the expression level of a polypeptide is not considered to be an agent that inhibits the activity of the polypeptide. In some embodiments, the activity of the Lin-28-like polypeptide (e.g., inhibition of the processing of a regulated primary microRNA transcript by Drosha, inhibition of the processing of a regulated precursor microRNA by Dicer) is inhibited by the polynucleotide or compound by at least 5%, at least 10%, at least 20%, at least 30%, at least 40%, at least 50%, at least 60%, at least 70%, at least 80%, at least 90%, at least 95%, at least 96%, at least 97%, at least 98%, at least 99%, or higher.

The compound may bind directly to the Lin-28-like polypeptide, inhibiting its activity, or it might indirectly inhibit the activity of the Lin-28-like polypeptide by binding to and regulating the activity of a biological molecule that regulates the activity of the Lin-28-like polypeptide.

A “Lin-28-like polypeptide” refers to a polypeptide having the ability to inhibit at least one of Drosha-mediated processing of particular primary microRNA transcripts (e.g., primary Let-7 microRNA transcripts) and Dicer-mediated processing of particular precursor microRNAs (e.g., precursor Let-7 microRNAs), an activity that has been ascribed to the Lin-28 and Lin-28-B polypeptides. In some embodiments, the Lin-28-like polypeptide is an animal polypeptide, including but not limited to a human polypeptide. The amino acid sequences of human Lin-28 and Lin-28-B are set forth in SEQ ID NO: 92 and 93, respectively. Lin-28 and Lin-28-B are RNA-binding proteins that comprise two retroviral-type CCHC zinc-finger motifs (corresponding to amino acids 138-154 and 160-176 of SEQ ID NO: 92 and amino acids 128-144 and 150-166 of SEQ ID NO: 93) and a cold-shock domain (corresponding to amino acids 41-112 of SEQ ID NO: 92 and amino acids 31-102 of SEQ ID NO: 93), which is a unique domain organization among all known RNA binding proteins (Moss et al. (1997) Cell 88:637-646). A Lin-28-like polypeptide can be a naturally occurring polypeptide from any type of organism or one that has been synthetically produced through recombination methods, mutagenesis, or chemical synthesis.

Without being bound to any theory or mechanism of action, it is believed that the Lin-28/Lin-28-B proteins interact directly with primary miRNA transcripts or precursor miRNAs through either the zinc-finger motifs, the cold-shock domain, or both domains of the polypeptides. The term “Lin-28-like polypeptide” can refer to variants and active fragments of Lin-28 or Lin-28B that retain the ability to inhibit at least one of Drosha processing of regulated primary microRNA transcripts and Dicer processing of regulated precursor microRNAs. In some embodiments, a “Lin-28-like polypeptide” refers to a polypeptide comprising an amino acid sequence having at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% sequence identity to SEQ ID NO: 92 or 93. In some embodiments, a Lin-28-like polypeptide comprises similar functional domains as the Lin-28 and Lin-28-B polypeptides. Accordingly, in some embodiments, a Lin-28-like polypeptide comprises at least one zinc-finger motif similar to the first or second zinc-finger motif found in Lin-28 or Lin-28-B. The first and second zinc-finger motifs of Lin-28 are set forth in SEQ ID NO: 94 and 95, respectively. The first and second zinc-finger motifs of Lin-28-B are set forth in SEQ ID NO: 96 and 97, respectively. Thus, in these embodiments, a Lin-28-like polypeptide is one comprising at least one zinc finger motif having an amino acid sequence having at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% sequence identity to SEQ ID NO: 94, 95, 96, or 97. In other embodiments, a Lin-28-like polypeptide comprises a cold-shock domain similar to the cold-shock domain of Lin-28 or Lin-28-B, which is set forth in SEQ ID NO: 98 and 99, respectively. In these embodiments, a Lin-28-like polypeptide is one comprising an amino acid sequence having at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% sequence identity to SEQ ID NO: 98 or 99. In some of these embodiments, the Lin-28-like polypeptide comprises at least one zinc-finger motif and a cold shock domain similar to these domains in Lin-28 and Lin-28-B. In these particular embodiments, a Lin-28-like polypeptide comprises at least one zinc-finger motif having an amino acid sequence having at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% sequence identity to SEQ ID NO: 94, 95, 96, or 97 and a cold shock domain having at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% sequence identity to SEQ ID NO: 98 or 99.

In some embodiments, the presently disclosed compounds can inhibit the interaction between the Lin-28-like polypeptide and at least one of a regulated primary microRNA transcript and a regulated precursor microRNA when introduced into a cell. As used herein, the terms “interact” and “interaction” refer to a transient or stable binding between two molecules. The two molecules may be directly bound to one another or bound indirectly through at least one intervening molecule. The ability of a compound to inhibit the interaction between a Lin-28-like-polypeptide and at least one of a regulated primary microRNA transcript and a regulated precursor microRNA can be measured using methods known in the art, including, but not limited to in vitro binding assays and UV cross-linking analysis (see, for example, Experimental Examples 1 and 2 and Myer, Fan & Steitz (1997) EMBO J. 16:2130-2139, which is herein incorporated by reference in its entirety). In some embodiments, the interaction between the Lin-28-like polypeptide and a regulated primary microRNA transcript or a regulated precursor microRNA is inhibited by the introduction of a compound by at least 5%, at least 10%, at least 20%, at least 30%, at least 40%, at least 50%, at least 60%, at least 70%, at least 80%, at least 90%, at least 95%, at least 96%, at least 97%, at least 98%, at least 99%, or higher.

Primary microRNA transcripts and precursor microRNAs that are regulated by Lin-28-like polypeptides, referred to herein as “Lin-28-like polypeptide-regulated primary microRNA transcripts” and “Lin-28-like polypeptide-regulated precursor microRNAs,” can be identified by overexpressing or ectopically expressing a Lin-28-like polypeptide in a cell and identifying those corresponding mature microRNA molecules whose expression/processing is reduced relative to those cells that do not express the Lin-28-like polypeptide or express it at lower levels using any method known to one of ordinary skill in the art that can measure the expression level or processing of one or more microRNAs, including a microRNA microarray format (see, for example, Thomson et al. (2004) Nature Methods 1:47-53, which is herein incorporated by reference in its entirety), quantitative PCR, Northern blots, or a cell-free Drosha or Dicer processing assay (see Experimental Example 1). Alternatively, primary microRNA transcripts and precursor microRNAs regulated by Lin-28-like polypeptides can be identified by reducing the expression of a Lin-28-like polypeptide using any technique known in the art, including but not limited to RNAi-mediated silencing with siRNA, and gene disruption techniques, followed by an analysis of the expression of a panel of mature miRNAs and the identification of those miRNAs present at increased levels upon reducing the expression of the Lin-28-like polypeptide.

In some embodiments, the regulated primary microRNA transcript comprises a primary Let-7 microRNA transcript or the regulated precursor microRNA comprises a precursor Let-7 microRNA. Data presented elsewhere herein demonstrate that during embryonic development, Let-7 microRNA processing is regulated at the level of Drosha processing and that Lin-28 and Lin-28B polypeptides are responsible for the inhibition of Drosha-mediated processing of primary Let-7 microRNA transcripts (see Experimental Example 1). Other data presented herein demonstrates that Lin-28 similarly inhibits Drosha-mediated processing of precursor miRNAs. Let-7 microRNAs have been identified in a wide array of organisms, from C. elegans to humans. The human let-7 miRNA family currently comprises 12 members, including Let-7a-1, Let-7a-2, Let-7a-3, Let-7b, Let-7c, Let-7d, Let-7e, Let-7f-1, Let-7f-2, Let-7g, Let-71, and miR-98. Other Let-7 family members from other species can be found on the online microRNA database miRBase (microrna.sanger.ac.uk/sequences/; Griffiths-Jones et al. (2008) Nucleic Acids Research 36(Database Issue):D154-D158; Griffiths-Jones et al. (2006) Nucleic Acids Research 34(Database Issue):D140-D144; Griffiths-Jones (2004) Nucleic Acids Research 32(Database Issue):D109-D111).

A. Polynucleotides

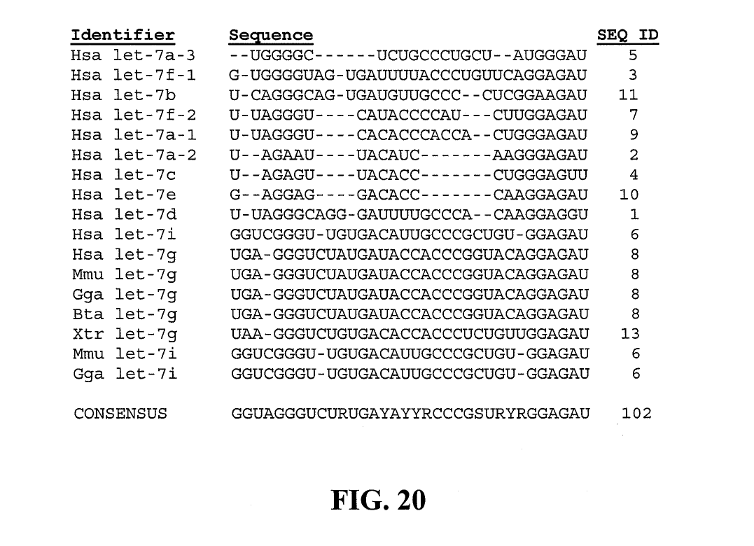

Data presented elsewhere herein demonstrates that Lin-28-like polypeptides inhibit the Drosha-mediated processing of particular primary microRNA transcripts, including but not limited to the Let-7 family of microRNAs. In addition, data is presented herein that demonstrates that Lin-28-like polypeptides inhibit Dicer-mediated processing of precursor miRNAs. Polynucleotides comprising the loop region sequence of these regulated primary microRNA transcripts or regulated precursor microRNAs (e.g., Let-7 family primary microRNA transcripts or pre-miRNAs) or active variants or fragments thereof can promote the processing of these regulated miRNAs. In some of these embodiments, the loop region sequence is from a Lin-28-like polypeptide-regulated primary microRNA transcript or Lin-28-like polypeptide-regulated precursor microRNA. Thus, in some embodiments, the present invention provides isolated polynucleotides comprising or encoding a nucleotide sequence corresponding to the loop region of Let-7 primary and precursor microRNAs or active variants or fragments thereof. Sequences of the loop regions of representative Let-7 microRNA family members are set forth in SEQ ID NOs 1-14 (see Table 1). A consensus sequence of some of the Let-7 family members is set forth in SEQ ID NO: 102 (see FIG. 20). Compositions of the invention include, but are not limited to, isolated polynucleotides comprising or encoding a nucleotide sequence having at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99% or more sequence identity to any one of the sequences set forth in SEQ ID NO: 1, 2, 3, 4, 5, 6, 7, 8, 9, 10, 11, 12, 13, 14, or 102, or an active fragment thereof, and wherein the polynucleotide promotes the processing of at least one of a regulated primary microRNA transcript by Drosha and a regulated precursor microRNA by Dicer when introduced into or expressed in a cell. In some embodiments, the polynucleotide or expression product thereof can not be processed to form a mature Let-7 miRNA. In other embodiments, the polynucleotide or expression product thereof can not be processed to form a mature endogenous miRNA molecule. In certain embodiments, the isolated polynucleotide comprises or encodes the nucleotide sequence set forth in SEQ ID NO: 102.

While not being bound to any theory or mechanism of action, it is believed the polynucleotides of the invention inhibit the activity of a Lin-28-like polypeptide by binding (directly or indirectly) to a Lin-28-like polypeptide and competing with primary microRNA transcripts or precursor microRNAs for the polypeptide. In the absence of the polynucleotides of the invention, these primary microRNA transcripts would not be processed by Drosha or the precursor microRNAs would not be processed by Dicer due to their association with the Lin-28-like polypeptide. Thus, the polynucleotides of the invention must be introduced or expressed in a sufficient amount to compete with regulated primary microRNA transcripts or regulated precursor microRNAs for binding to Lin-28-like polypeptides and to promote the processing of these regulated primary microRNA transcripts by Drosha or the processing of the regulated precursor microRNAs by Dicer.

The terms “nucleic acid,” “polynucleotide,” or “oligonucleotide” generally are used herein in their art-accepted manners to refer to a polymer of nucleotides. As used herein, an oligonucleotide is typically less than 100 nucleotides in length. Polynucleotides can be single-stranded (with or without a secondary structure, e.g., hairpin) or double-stranded. Naturally occurring nucleic acids include deoxyribonucleic acid (DNA) and ribonucleic acid (RNA). The polynucleotide or oligonucleotide may include natural nucleosides (e.g., adenosine, thymidine, guanosine, cytidine, uridine, deoxyadenosine, deoxythymidine, deoxyguanosine, and deoxycytidine), or synthetic nucleosides, such as, nucleoside analogs (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), and/or nucleosides comprising chemically or biologically modified bases, such as those ribonucleosides that are substituted at the 2′ position, for example, with an alkyl or alkyloxy group (e.g., methylated bases, such as those that are 2′-O-methylated, and 2′-β-methoxyethylated) or a fluoro group, intercalated bases, and/or modified sugars (e.g., 2′-fluororibose, ribose, 2′-deoxyribose, arabinose, and hexose). The phosphate groups in a polynucleotide or oligonucleotide are typically considered to form the internucleoside backbone of the polymer. In naturally occurring nucleic acids (e.g., DNA or RNA), the backbone linkage is via a 3′ to 5′ phosphodiester bond. Polynucleotides and oligonucletides containing modified backbones or non-naturally occurring internucleoside linkages, however, also can be used in the presently disclosed subject matter. Such modified backbones include backbones that have a phosphorus atom in the backbone and others that do not have a phosphorus atom in the backbone. Examples of modified linkages include, but are not limited to, phosphorothioate and 5′-N-phosphoramidite linkages. The polynucleotide also may be a locked nucleic acid molecule, which comprises a ribonucleotide that has a methylene bridge between the 2′-oxygen atom and the 4′-carbon atom. See, for example, Kurreck et al. (2002) Nucleic Acids Res. 30: 1911-1918; Elayadi et al. (2001) Curr. Opinion Invest. Drugs 2: 558-561; Orum et al. (2001) Curr. Opinion Mol. Ther. 3: 239-243; Koshkin et al. (1998) Tetrahedron 54: 3607-3630; Obika et al. (1998) Tetrahedron Lett. 39: 5401-5404. In other embodiments, the polynucleotide comprises peptide nucleic acid (PNA) moieties that comprise a base bonded to an amino acid residue as the backbone unit (Nielson (1999) Methods Enzymol. 313: 156-164; Elayadi, et al. (2001) Curr. Opinion Invest. Drugs 2: 558-561; Braasch et al. (2002) Biochemistry 41: 4503-4509, Nielsen et al. (1991) Science 254: 1497-1500). In other embodiments, the polynucleotide comprises at least one morpholino phosphoroamidate nucleotide moiety (Heasman (2002) Dev. Biol. 243: 209-214), at least one cyclohexene nucleotide moiety (Wang et al. (2000) J. Am. Chem. Soc. 122: 8595-8602, Verbeure et al. (2001) Nucleic Acids Res. 29: 4941-4947), or at least one tricyclo nucleotide moiety (Steffens et al. (1997) J. Am. Chem. Soc. 119: 11548-11549, Renneberg et al. (2002) J. Am. Chem. Soc. 124: 5993-6002. Polynucleotides and oligonucleotides need not be uniformly modified along the entire length of the molecule. For example, different nucleotide modifications, different backbone structures, and the like, may exist at various positions in the polynucleotide or oligonucleotide. Any of the polynucleotides described herein may utilize these modifications.

Thus, in some embodiments, the polynucleotide used to inhibit the activity of a Lin-28-like polypeptide is a single-stranded RNA oligonucleotide. In certain embodiments, the polynucleotide comprises modified nucleic acids (such as those defined elsewhere herein) to impart stability to the polynucleotide. In some of these embodiments, the polynucleotide comprises 2′-O-methylated ribonucleotides.

The invention encompasses isolated or substantially purified polynucleotide compositions. An “isolated” or “purified” polynucleotide, or biologically active portion thereof, is substantially or essentially free from components that normally accompany or interact with the polynucleotide as found in its naturally occurring environment. Thus, an isolated or purified polynucleotide is substantially free of other cellular material, or culture medium when produced by recombinant techniques, or substantially free of chemical precursors or other chemicals when chemically synthesized. In some embodiments, an “isolated” polynucleotide is free of sequences that naturally flank the polynucleotide (i.e., sequences located at the 5′ and 3′ ends of the polynucleotide) in the genomic DNA of the organism from which the polynucleotide is derived. In some embodiments, the presently disclosed isolated polynucleotides further comprise an additional 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 150, 200, 300, 400, 500, 600, 700, 800, 900, 1000, 1500, 2000, 3000, 4000, 5000 nucleotides or more on either the 5′ end, the 3′ end, or both ends of the genomic or RNA sequence.

Fragments and variants of the disclosed polynucleotides are also encompassed by the present invention. By “fragment” is intended a portion of the polynucleotide and include active fragments that retain the biological activity of the polynucleotide of the invention. Therefore, an active fragment can exhibit at least one of the following activities: promotes the processing of at least one of a regulated primary microRNA transcript by Drosha and a regulated precursor microRNA by Dicer (e.g., through the inhibition of the activity of a Lin-28-like polypeptide), and interacts with a Lin-28-like polypeptide. Alternatively, fragments of a polynucleotide that are useful as hybridization probes or PCR primers need not retain this biological activity. Thus, fragments of a nucleotide sequence may range from at least about 5 nucleotides, about 10 nucleotides, about 20 nucleotides, and up to the full-length polynucleotide of the invention.

Thus, a fragment of the polynucleotides of the invention may be biologically active or it may be a fragment that can be used as a hybridization probe or PCR primer using methods disclosed below. An active fragment of the disclosed sequences can be prepared by isolating a portion of one of the polynucleotides of the invention (e.g., by recombinant expression in vitro) or chemically synthesizing the polynucleotide and assessing the activity of the polynucleotide. Polynucleotides that are active fragments of the polynucleotides of the invention have a nucleotide sequence comprising at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33 contiguous nucleotides of the sequences of the invention, or up to the number of nucleotides present in a full-length polynucleotide disclosed herein (for example, 34 nucleotides for SEQ ID NO: 12).

“Variants” is intended to mean substantially similar sequences. A variant comprises a polynucleotide having deletions (i.e., truncations) at the 5′ and/or 3′ end; deletion and/or addition of one or more nucleotides at one or more internal sites in comparison to a particular polynucleotide of the invention; and/or substitution of one or more nucleotides at one or more sites in the polynucleotide of the invention. Naturally occurring allelic variants such as these can be identified with the use of well-known molecular biology techniques, for example, with polymerase chain reaction (PCR) and hybridization techniques as outlined below. Variant polynucleotides also include synthetically derived polynucleotides, such as those generated, for example, by using site-directed mutagenesis (but which still retain the activity of the polynucleotides of the invention). Generally, variants of a particular polynucleotide of the invention will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular polynucleotide as determined by sequence alignment programs and parameters as described elsewhere herein.

The presently disclosed isolated polynucleotides can encode a loop region nucleotide sequence. In some embodiments, the isolated polynucleotide encodes a nucleotide sequence having at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99% or more sequence identity to any one of the sequences set forth in SEQ ID NO: 1, 2, 3, 4, 5, 6, 7, 8, 9, 10, 11, 12, 13, 14, or 102 or an active fragment thereof, and wherein the polynucleotide promotes the processing of at least one of a regulated primary microRNA transcript by Drosha and a regulated precursor microRNA by Dicer when introduced into or expressed in a cell. In some of these embodiments, the polynucleotide can not be processed to form a mature let-7 miRNA when introduced into or expressed in a cell. In other embodiments, the polynucleotide can not be processed to form a mature endogenous miRNA. In some embodiments, the polynucleotide encodes the nucleotide sequence set forth in SEQ ID NO: 102.

In these embodiments, the polynucleotide comprises a coding sequence for the disclosed sequences. A “coding sequence” refers to a nucleotide sequence (e.g., DNA) that encodes a specific RNA or polypeptide.

In some of these embodiments, the isolated polynucleotides that encode for the disclosed RNA sequences comprise a nucleotide sequence having at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99% or more sequence identity to any one of the sequences set forth in SEQ ID NO: 78, 79, 80, 81, 82, 83, 84, 85, 86, 87, 88, 89, 90, or 91, or an active fragment thereof.

In some embodiments, the isolated polynucleotides of the invention or an expression product thereof comprise a stem-loop structure and can be recognized and processed by Dicer or Drosha, wherein the loop of the stem-loop structure has the loop sequence of a regulated primary microRNA transcript, a regulated precursor microRNA, or an active variant or fragment thereof. In particular embodiments, the loop sequence is from a loop of a Lin-28-like polypeptide-regulated primary microRNA transcript, a Lin-28-like polypeptide-regulated precursor microRNA or an active variant or fragment thereof. In some of these embodiments, the loop has a nucleotide sequence having at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99% or more sequence identity to any one of the sequences set forth in SEQ ID NO: 1, 2, 3, 4, 5, 6, 7, 8, 9, 10, 11, 12, 13, 14, or 102 or an active fragment thereof. In these embodiments, the stem of the stem-loop structures comprise a microRNA. The processing of the polynucleotide or expression product thereof by Drosha or Dicer is inhibited by a Lin-28-like polypeptide. Thus, the miRNA of the stem-loop structure will be selectively processed by Drosha or Dicer and the target polynucleotide suppressed in those cells that have a relatively low level of Lin-28-like polypeptides. In some embodiments, the loop has the nucleotide sequence set forth in SEQ ID NO: 102.

In some embodiments, the loop has a sequence that is heterologous to the sequence of the stem of the stem-loop structure. As used herein, “heterologous” in reference to a sequence is a sequence that originates from a foreign species, or, if from the same species, is substantially modified from its native form in composition and/or genomic locus by deliberate human intervention. For example, a stem sequence of a stem-loop structure (e.g., of a primary microRNA transcript or a precursor microRNA) is heterologous to the loop sequence of the same stem-loop structure if the stem sequence is from a species different from the species from which the loop sequence was derived, or, if from the same/analogous species, one or both are substantially modified from their original form.

In some embodiments, the loop sequence interacts with a Lin-28-like polypeptide (e.g., Lin-28, Lin-28B).

In some embodiments, a target polynucleotide of the miRNA of the stem-loop structure is a disease-causing gene. As used herein, a “disease-causing gene” is a gene that contributes to the initiation, progression, or maintenance of a pathological state. A disease-causing gene can refer to a gene that is overexpressed or mutated in such a manner as to activate the encoded polypeptide, or wherein the polypeptide encoded by the gene is modified in such a manner as to contribute to the disease state. In certain embodiments, the disease-causing gene comprises an oncogene. The term “oncogene” is used herein in accordance with its art-accepted meaning to refer to those polynucleotide sequences that encode a gene product that contributes to cancer initiation or progression. The term “oncogene” encompasses proto-oncogenes, which are genes that do not contribute to carcinogenesis under normal circumstances, but that have been mutated, overexpressed, or otherwise activated in such a manner as to function as an oncogene. Non-limiting examples of oncogenes include growth factors or mitogens (e.g., c-Sis), receptor tyrosine kinases (e.g., epidermal growth factor receptor (EGFR), platelet-derived growth factor receptor (PDGFR), vascular endothelial growth factor receptor (VEGFR), HER2/neu), cytoplasmic tyrosine kinases (e.g., src, Abl), cytoplasmic serine/threonine kinases (e.g., raf kinase, cyclin-dependent kinases), regulatory GTPases (e.g., ras), and transcription factors (e.g., myc).

In some embodiments, the reduction in the expression level of a target polynucleotide of the microRNA suppresses the growth or viability of the cells in which the polynucleotide comprising or encoding the stem-loop structure comprising the miRNA has been introduced or expressed. Non-limiting examples of target polynucleotides the suppression of which would result in growth suppression or a decrease in cellular viability can be found, for example, in Schlabach et al. (2008) Science 319(5863):620-624, which is herein incorporated by reference in its entirety.

In some of these embodiments, the polynucleotide or expression product thereof having the stem-loop structure can be recognized and processed by Drosha and is essentially a primary miRNA transcript as defined elsewhere herein, whereby the stem-loop structure is cleaved from the polynucleotide sequence, similar to the processing of endogenous primary microRNA transcripts. Flanking sequences that are found on either side of the stem-loop structure of particular sequences or structure can be used or added to the polynucleotide to allow the polynucleotide to be recognized and cleaved by Drosha. Such sequence and structural preferences of Drosha are known in the art (see, for example, Han et al. (2006) Cell 125:887-901 and Helvik et al. (2007) Bioinformatics 23:142-149, both of which are herein incorporated by reference in their entirety). In some embodiments, the polynucleotide or expression product thereof comprising the stem-loop structure can be recognized and processed by Dicer and is essentially a pre-miRNA as defined elsewhere herein, whereby the stem-loop structure is processed to generate a double-stranded miRNA duplex, similar to the processing of endogenous precursor miRNAs. Dicer binds to short 3′ overhanging end of small RNAs that mimic the short (about 2 nucleotide) 3′ overhang on endogenous pre-miRNAs created by Drosha cleavage of the pri-miRNA transcript.

The polynucleotides of the invention can be used to isolate variants of the presently disclosed polynucleotide sequences from any organism. In this manner, methods such as PCR, hybridization, and the like can be used to identify such sequences based on their sequence homology to the sequences set forth herein. Sequences isolated based on their sequence identity to the entire polynucleotides sequences set forth herein or to variants and fragments thereof are encompassed by the present invention. In a PCR approach, oligonucleotide primers can be designed for use in PCR reactions to amplify corresponding polynucleotide sequences from cDNA or genomic DNA extracted from any organism of interest. Methods for designing PCR primers and PCR cloning are generally known in the art and are disclosed in Sambrook et al. (1989) Molecular Cloning: A Laboratory Manual (2d ed., Cold Spring Harbor Laboratory Press, Plainview, N.Y.). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially-mismatched primers, and the like.

B. Assays to Measure Drosha-Mediated and Dicer-Mediated Processing

Drosha-mediated processing of a regulated primary microRNA transcript can be measured in vitro or in vivo through the measurement of the levels of pre-miRNA, miRNA duplexes, or mature miRNA. Cells which exhibit enhanced Drosha processing are expected to have higher levels of pre-miRNA, miRNA duplexes, or mature miRNA relative to a control cell. In some embodiments, these cells may also exhibit reduced levels of primary miRNA transcripts relative to a control cell. Similarly, Dicer-mediated processing of regulated precursor microRNAs can be measured in vitro or in vivo through the measurement of the levels of miRNA duplexes or mature miRNAs, the levels of which are expected to be higher in cells with enhanced Dicer activity. In some embodiments, these cells may also exhibit reduced levels of pre-miRNAs relative to a control cell. The levels of the miRNA or various precursors can be measured using techniques known in the art, including, but not limited to, PCR (e.g., quantitative RT-PCR), Northern blots, and microRNA microarrays (see, for example, Thomson et al. (2004) Nature Methods 1:47-53).

Alternatively, the level of Drosha or Dicer processing can be indirectly measured by monitoring the expression of a target polynucleotide (either the target mRNA or polypeptide encoded thereby) that is known to be regulated by the mature miRNA using techniques known in the art. For example, PCR (e.g., quantitative RT-PCR), Northern blots, or microarrays can be used to measure the level of target mRNAs and Western blots or an ELISA, for example, can be used to measure the level of target polypeptides. In addition, assays that measure the activity of target polypeptides can be used as an indirect measure of miRNA production.

It should be noted that the levels of miRNA and the expression of target polynucleotides can also be affected by various downstream mechanisms, including, but not limited to other post-transcriptional and translational regulatory mechanisms. Thus, the most direct measurement of the ability of a nucleotide sequence to promote Drosha processing of a particular regulated primary microRNA transcript is the levels of pre-miRNA in a cell. In vitro assays that measure Drosha activity such as the cell-free Drosha processing assay described elsewhere herein (see, for example, Experimental Example 1) and known in the art (Lee et al. (2002) EMBO J 21:4663-4670, which is herein incorporated by reference in its entirety), can also be used to assess the ability of a compound (e.g., polynucleotide) to promote Drosha processing. The cell-free Drosha processing assay involves preparing nuclear extracts from cells and incubating the extracts with a primary miRNA transcript in the presence or absence of the compound in question, resolving the pri-microRNA products on a sequencing gel and detecting the presence of processed pre-miRNA products (e.g., using radiolabeled primers specific for the pre-miRNA product). In some embodiments, the cell-free Drosha processing assay can further comprise immunoprecipitated Drosha protein (see, for example, Experimental Example 1). A similar experimental design can be used to measure the effect of a polynucleotide on Dicer processing in vitro, wherein cellular or cytoplasmic extracts are incubated with a precursor miRNA and processed miRNA duplexes (or mature miRNA or miRNA* sequences) are detected, such as the Dicer processing assay described elsewhere herein (see Experimental Example 2). The cell-free Dicer processing assay can further comprise recombinant Dicer or Dicer immunoprecipitates.

To determine if the Dicer-mediated processing of a given precursor miRNA or the Drosha-mediated processing of a given primary miRNA transcript is inhibited by a Lin-28-like polypeptide, various assays can be utilized. For example, the ability of a Lin-28-like polypeptide to inhibit the Drosha-mediated processing of a primary miRNA transcript or a miRNA-comprising polynucleotide can be measured in a cell-free Drosha processing assay known in the art or described herein. Likewise, the Dicer-mediated processing of a given polynucleotide can be measured using a cell-free Dicer processing assay known in the art or described herein. For example, the Lin-28-like polypeptide can be overexpressed or silenced within cells from which the nuclear or cytoplasmic extract is prepared. Alternatively, recombinantly produced or immunoprecipitated Lin-28-like polypeptide can be added to the Drosha or Dicer assay. The ability of a Lin-28-like polypeptide to inhibit the Drosha-mediated or Dicer-mediated processing of a polynucleotide can also be assayed by measuring the generation of precursor miRNAs or mature miRNAs using, for example, quantitative PCR in cells expressing the primary miRNA transcript, precursor miRNA, or miRNA-comprising polynucleotide wherein the Lin-28-like polypeptide has been overexpressed or silenced and comparing the results to an appropriate control.

C. Expression Cassettes

The presently disclosed subject matter also provides for expression cassettes comprising a polynucleotide encoding a nucleotide sequence corresponding to the loop region of microRNAs comprised within regulated primary microRNA transcripts or regulated precursor microRNAs. In some embodiments, the expression cassette comprises a polynucleotide encoding a nucleotide sequence having at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99% or more sequence identity to any one of the sequences set forth in SEQ ID NO: 1, 2, 3, 4, 5, 6, 7, 8, 9, 10, 11, 12, 13, 14, or 102, or an active fragment thereof, and wherein the polynucleotide promotes the processing of at least one of a regulated primary microRNA transcript by Drosha and a regulated precursor by Dicer when expressed in a cell. In some of these embodiments, the polynucleotide or an expression product thereof can not be processed to form a mature Let-7 miRNA when introduced into or expressed in a cell. In certain embodiments, the polynucleotide or expression product thereof can not be processed to form a mature endogenous miRNA when introduced into or expressed in a cell. In some embodiments, the expression cassette comprises a polynucleotide encoding the nucleotide sequence set forth in SEQ ID NO: 102.

In some of these embodiments, the isolated polynucleotides that encode for the disclosed RNA sequences comprise a nucleotide sequence having at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99% or more sequence identity to any one of the sequences set forth in SEQ ID NO: 78, 79, 80, 81, 82, 83, 84, 85, 86, 87, 88, 89, 90, or 91, or an active fragment thereof.

In some embodiments, the expression cassettes of the invention comprise a polynucleotide having a stem-loop structure that is recognized and processed by Drosha or Dicer, wherein the loop of the stem-loop structure has the loop sequence of a regulated primary microRNA transcript, a regulated precursor microRNA, or an active variant or fragment thereof. In particular embodiments, the loop sequence is from a loop of a Lin-28-like polypeptide-regulated primary microRNA transcript, a Lin-28-like polypeptide-regulated precursor microRNA, or an active variant or fragment thereof. In some of these embodiments, the loop has a nucleotide sequence having at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99% or more sequence identity to any one of the sequences set forth in SEQ ID NO: 1, 2, 3, 4, 5, 6, 7, 8, 9, 10, 11, 12, 13, 14, or 102, or an active fragment thereof. The stem of the stem-loop structures comprise a microRNA. The processing of the polynucleotide or expression product thereof by Drosha or Dicer is inhibited by a Lin-28-like polypeptide. In certain embodiments, the loop sequence is heterologous to the stem sequence. In some embodiments, the target polynucleotide of the miRNA comprises a disease-causing gene, such as an oncogene. In certain embodiments, the loop sequence interacts with a Lin-28-like polypeptide (e.g., Lin-28, Lin-28B). In some embodiments, the loop has the nucleotide sequence set forth in SEQ ID NO: 102.

The expression cassette comprises one or more regulatory sequences operably linked to the polynucleotide encoding the active polynucleotide. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. See, for example, Goeddel (1990) in Gene Expression Technology: Methods in Enzymology 185 (Academic Press, San Diego, Calif.). Regulatory sequences include those that direct constitutive expression of a nucleotide sequence in many types of host cells and those that direct expression of the nucleotide sequence only in certain host cells (e.g., tissue-specific regulatory sequences). Regulatory sequences may include promoters, translation leader sequences, introns, and polyadenylation recognition sequences.

“Operably linked” is intended to mean a functional linkage between two or more elements. For example, an operable linkage between a polynucleotide that encodes a loop region sequence or a stem-loop structure comprising the same and a regulatory sequence (i.e., a promoter) is a functional link that allows for expression of the loop region sequence or stem-loop structure comprising the same. Operably linked elements may be contiguous or non-contiguous. Polynucleotides may be operably linked to regulatory sequences in sense or antisense orientation. The regulatory regions (i.e., promoters, transcriptional regulatory regions, and translational termination regions) and/or the presently disclosed polynucleotides may be native/analogous to the host cell or to each other. Alternatively, the regulatory regions and/or the presently disclosed polynucleotides may be heterologous to the host cell or to each other. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide.

“Promoter” refers to a polynucleotide capable of controlling the expression of a polynucleotide. In general, the polynucleotide to be transcribed is located 3′ to a promoter sequence. The promoter sequence may comprise proximal and more distal upstream elements; the latter elements often referred to as enhancers. Accordingly, an “enhancer” is a polynucleotide, which can stimulate promoter activity, and may be an innate element of the promoter or a heterologous element inserted to enhance the level or tissue-specificity of a promoter. Promoters may be derived in their entirety from a native gene, or be composed of different elements derived from different promoters found in nature, or even comprise synthetic nucleotide segments. It is recognized that since in most cases the exact boundaries of regulatory sequences have not been completely defined, polynucleotide fragments of different lengths may have identical promoter activity.

In some embodiments, the promoter utilized to direct intracellular expression of a loop region sequence or a stem-loop structure comprising the same is a promoter recognized by RNA polymerase II (Pol II). In other embodiments, the expression of a loop region sequence or a stem-loop structure comprising the same is directed by a promoter recognized by a RNA polymerase III (Pol III), such as the U6 promoter (e.g., human, murine) or H1 promoter (e.g., human, murine).

It will be appreciated by those skilled in the art that the design of the expression cassette can depend on such factors as the choice of the host cell to be transformed, the level of expression of the presently disclosed polynucleotides, and the like. Such expression cassettes typically include one or more appropriately positioned sites for restriction enzymes, to facilitate introduction of the nucleic acid into a vector. It will further be appreciated that appropriate promoter and/or regulatory elements can readily be selected to allow expression of the presently disclosed polynucleotides in the cell of interest. In some embodiments, a promoter can be selected that confers inducible, constitutive, environmentally- or developmentally-regulated, or cell- or tissue-specific/selective expression. Chemical-regulated promoters can be used to express the presently disclosed polynucleotide through the application of an exogenous chemical regulator. Depending upon the objective, the promoter may be a chemical-inducible promoter, where application of the chemical induces gene expression, or a chemical-repressible promoter, where application of the chemical represses gene expression. Other chemical-regulated promoters of interest include steroid-responsive promoters (see, for example, the glucocorticoid-inducible promoter in Schena et al. (1991) Proc. Natl. Acad. Sci. USA 88:10421-10425 and McNellis et al. (1998) Plant J. 14(2):247-257) and tetracycline-inducible and tetracycline-repressible promoters (see, for example, Gatz et al. (1991) Mol. Gen. Genet. 227:229-237, and U.S. Pat. Nos. 5,814,618 and 5,789,156), herein incorporated by reference.

Additional regulatory sequences found within expression cassettes include a 3′ non-coding region. The “3′ non-coding region” or “terminator region” refers to DNA or RNA sequences located downstream of a coding sequence and may include polyadenylation recognition sequences and other sequences encoding regulatory signals capable of affecting mRNA processing or gene expression. The polyadenylation signal is usually characterized by effecting the addition of polyadenylic acid tracts to the 3′ end of the mRNA precursor. The use of different 3′ non-coding sequences is exemplified by Ingelbrecht et al. (1989) Plant Cell 1:671-680.

Regulatory sequences can be provided by viral regulatory elements. For example, commonly used promoters are derived from polyoma, Adenovirus 2, cytomegalovirus, and Simian Virus 40. For other suitable expression systems for both prokaryotic and eukaryotic cells, see Chapters 16 and 17 of Sambrook et al. (1989) Molecular Cloning: A Laboratory Manual (2d ed., Cold Spring Harbor Laboratory Press, Plainview, N.Y.). See, Goeddel (1990) in Gene Expression Technology: Methods in Enzymology 185 (Academic Press, San Diego, Calif.).

The expression cassette can also comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT). Additional selectable markers include phenotypic markers such as β-galactosidase and fluorescent proteins such as green fluorescent protein (GFP) (Su et al. (2004) Biotechnol Bioeng 85:610-9 and Fetter et al. (2004) Plant Cell 16:215-28), cyan florescent protein (CYP) (Bolte et al. (2004) J. Cell Science 117:943-54 and Kato et al. (2002) Plant Physiol 129:913-42), and yellow florescent protein (PhiYFP™ from Evrogen, see, Bolte et al. (2004) J. Cell Science 117:943-54). For additional selectable markers, see generally, Yarranton (1992) Curr. Opin. Biotech. 3:506-511; Christopherson et al. (1992) Proc. Natl. Acad. Sci. USA 89:6314-6318; Yao et al. (1992) Cell 71:63-72; Reznikoff (1992) Mol. Microbiol. 6:2419-2422; Barkley et al. (1980) in The Operon, pp. 177-220; Hu et al. (1987) Cell 48:555-566; Brown et al. (1987) Cell 49:603-612; Figge et al. (1988) Cell 52:713-722; Deuschle et al. (1989) Proc. Natl. Acad. Aci. USA 86:5400-5404; Fuerst et al. (1989) Proc. Natl. Acad. Sci. USA 86:2549-2553; Deuschle et al. (1990) Science 248:480-483; Gossen (1993) Ph.D. Thesis, University of Heidelberg; Reines et al. (1993) Proc. Natl. Acad. Sci. USA 90:1917-1921; Labow et al. (1990) Mol. Cell. Biol. 10:3343-3356; Zambretti et al. (1992) Proc. Natl. Acad. Sci. USA 89:3952-3956; Baim et al. (1991) Proc. Natl. Acad. Sci. USA 88:5072-5076; Wyborski et al. (1991) Nucleic Acids Res. 19:4647-4653; Hillenand-Wissman (1989) Topics Mol. Struc. Biol. 10:143-162; Degenkolb et al. (1991) Antimicrob. Agents Chemother. 35:1591-1595; Kleinschnidt et al. (1988) Biochemistry 27:1094-1104; Bonin (1993) Ph.D. Thesis, University of Heidelberg; Gossen et al. (1992) Proc. Natl. Acad. Sci. USA 89:5547-5551; Oliva et al. (1992) Antimicrob. Agents Chemother. 36:913-919; Hlavka et al. (1985) Handbook of Experimental Pharmacology, Vol. 78 (Springer-Verlag, Berlin); Gill et al. (1988) Nature 334:721-724. Such disclosures are herein incorporated by reference.

The above list of selectable marker genes is not meant to be limiting. Any selectable marker gene can be used in the present invention.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

In some embodiments, the expression cassettes can be used for in vitro transcription of the coding sequence into an RNA oligonucleotide. In vitro transcription may be performed using a variety of available systems including the T7, SP6, and T3 promoter/polymerase systems (e.g., those available commercially from Promega, Clontech, New England Biolabs, and the like). Vectors including the T7, SP6, or T3 promoter are well known in the art and can readily be modified to direct transcription of the presently disclosed polynucleotides.

Such expression cassettes can be contained in a vector which allow for the introduction of the expression cassette into a cell. In specific embodiments, the vector allows for autonomous replication of the expression cassette in a cell or may be integrated into the genome of a cell. Such vectors are replicated along with the host genome (e.g., nonepisomal mammalian vectors). In general, expression vectors of utility in recombinant DNA techniques are often in the form of plasmids (vectors). However, the invention is intended to include such other forms of expression vectors, such as viral vectors (e.g., replication defective retroviruses, adenoviruses, and adeno-associated viruses).

D. Methods of Expression

According to the present invention, polynucleotides or expression cassettes can be introduced into a cell. “Introducing” is intended to mean presenting to the cell the polynucleotide in such a manner that the sequence gains access to the interior of the cell. The methods of the invention do not depend on a particular method for introducing a sequence into a cell, only that the polynucleotide gains access to the interior of the cell. Methods for introducing polynucleotides into cells are known in the art including, but not limited to, stable transformation methods, transient transformation methods, and virus-mediated methods.

“Stable transformation” is intended to mean that the polynucleotide introduced into a cell integrates into the genome of the cell and is capable of being inherited by the progeny thereof “Transient transformation” is intended to mean that a polynucleotide is introduced into the cell and does not integrate into the genome of the cell.

Exemplary art-recognized techniques for introducing foreign polynucleotides into a host cell, including calcium phosphate or calcium chloride co-precipitation, DEAE-dextran-mediated transfection, lipofection, particle gun, or electroporation and viral vectors. Suitable methods for transforming or transfecting host cells can be found in U.S. Pat. No. 5,049,386, U.S. Pat. No. 4,946,787; and U.S. Pat. No. 4,897,355, Sambrook et al. (1989) Molecular Cloning: A Laboratory Manual (2d ed., Cold Spring Harbor Laboratory Press, Plainview, N.Y.) and other standard molecular biology laboratory manuals. One of skill will recognize that depending on the method by which a polynucleotide is introduced into a cell, the polynucleotide can be stably incorporated into the genome of the cell, replicated on an autonomous vector or plasmid, or present transiently in the cell. In some embodiments, transient expression may be desired. In those cases, standard transient transformation techniques may be used. Such methods include, but are not limited to viral transformation methods, and microinjection of DNA or RNA, as well other methods well known in the art.

Viral vector delivery systems include DNA and RNA viruses, which have either episomal or integrated genomes after delivery to the cell. For a review of viral vector procedures, see Anderson (1992) Science 256:808-813; Haddada et al. (1995) Current Topics in Microbiology and Immunology Doerfler and Bohm (eds); and Yu et al. (1994) Gene Therapy 1:13-26.

Conventional viral based systems for the delivery of the presently disclosed polynucleotides could include retroviral, lentivirus, adenoviral, adeno-associated and herpes simplex virus vectors for gene transfer. Viral vectors are currently the most efficient and versatile method of gene transfer in target cells and tissues. Integration in the host genome is possible with the retrovirus, lentivirus, and adeno-associated virus gene transfer methods, often resulting in long term expression of the inserted transgene.

Viral vectors can be delivered in vivo by administration to an individual patient, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient, followed by reimplantation of the cells into a patient, usually after selection for cells which have incorporated the vector.

Host organisms containing the introduced polynucleotide are referred to as “transgenic” organisms. By “host cell” is meant a cell that contains an introduced polynucleotide construct and supports the replication and/or expression of the construct. Host cells may be prokaryotic cells such as E. coli, or eukaryotic cells such as fungi, yeast, insect, amphibian, nematode, or mammalian cells. Mammalian cells of interest include cells from humans or from agricultural or domestic animals. Alternatively, the host cells are monocotyledonous or dicotyledonous plant cells. Examples of methods of plant transformation include Agrobacterium-mediated transformation (De Blaere et al. (1987) Meth. Enzymol. 143:277) and particle-accelerated or “gene gun” transformation technology (Klein et al. (1987) Nature (London) 327:70-73; U.S. Pat. No. 4,945,050), among others.

The skilled artisan will recognize that different independent transformation events will result in different levels and patterns of expression (Jones et al. (1985) EMBO J. 4:2411-2418; De Almeida et al. (1989) Mol. Gen. Genetics 218:78-86), and thus that multiple events may have to be screened in order to obtain cells displaying the desired expression level and pattern. Such screening may be accomplished by PCR or Southern analysis of DNA to determine if the introduced polynucleotide is present in complete form, and then northern analysis or RT-PCR to determine if the expected RNA is indeed expressed.

E. Pharmaceutical Compositions

The compounds (e.g., polynucleotides and expression cassettes) described herein are useful in mammalian tissue culture systems, in animal studies, and for therapeutic purposes. The presently disclosed compositions can be formulated for delivery, i.e., administering to the subject, by any available route including, but not limited, to parenteral (e.g., intravenous), intradermal, subcutaneous, oral, nasal, bronchial, opthalmic, transdermal (topical), transmucosal, rectal, and vaginal routes. In some embodiments, the route of delivery is intravenous, parenteral, transmucosal, nasal, bronchial, vaginal, or oral.

The presently disclosed pharmaceutical compositions also can include a compound with a pharmaceutically acceptable carrier. As used herein the term “pharmaceutically acceptable carrier” includes solvents, dispersion media, coatings, antibacterial and antifungal agents, isotonic and absorption delaying agents, and the like, compatible with pharmaceutical administration. Supplementary active compounds also can be incorporated into the compositions.

The presently disclosed subject matter contemplates pharmaceutical compositions comprising a pharmaceutical carrier and isolated polynucleotides (or expression cassettes comprising the same) comprising or encoding a nucleotide sequence of a loop region of regulated primary microRNA transcripts or loop regions of regulated precursor microRNAs or active variants or fragments thereof that promote the processing of at least one of primary microRNA transcripts by Drosha and precursor microRNAs by Dicer. In some of these embodiments, the loop region sequence is from a Lin-28-like polypeptide-regulated primary microRNA transcript or Lin-28-like polypeptide-regulated precursor microRNA. In particular embodiments, the isolated polynucleotide comprises or encodes a nucleotide sequence having at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99% or more sequence identity to any one of the sequences set forth in SEQ ID NO: 1, 2, 3, 4, 5, 6, 7, 8, 9, 10, 11, 12, 13, 14, or 102, or an active fragment thereof, and wherein the polynucleotide promotes the processing of at least one of a regulated primary microRNA transcript by Drosha and a regulated precursor miRNA by Dicer when introduced into or expressed in a cell. In some of these embodiments, the polynucleotide or an expression product thereof can not be processed to form a mature Let-7 miRNA when introduced into or expressed in a cell. In certain embodiments, the polynucleotide or expression product thereof can not be processed to form a mature endogenous miRNA. In some embodiments, the polynucleotide encodes the nucleotide sequence set forth in SEQ ID NO: 102.

In some of these embodiments, the isolated polynucleotide of the pharmaceutical composition or an expression product thereof comprises a stem-loop structure and can be recognized and processed by Dicer or Drosha, wherein the loop of the stem-loop structure has the loop sequence of a regulated primary microRNA transcript, a regulated precursor microRNA, or an active variant or fragment thereof. In particular embodiments, the loop sequence is from a loop of a Lin-28-like polypeptide-regulated primary microRNA transcript, a Lin-28-like polypeptide-regulated precursor microRNA, or an active variant or fragment thereof. In some of these embodiments, the loop has a nucleotide sequence having at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99% or more sequence identity to any one of the sequences set forth in SEQ ID NO: 1, 2, 3, 4, 5, 6, 7, 8, 9, 10, 11, 12, 13, 14, or 102, or an active fragment thereof. In these embodiments, the stem of the stem-loop structures comprises a microRNA. The processing of the polynucleotide or expression product thereof by Drosha or Dicer is inhibited by a Lin-28-like polypeptide. Thus, the miRNA of the stem-loop structure will be selectively processed by Drosha or Dicer and the target polynucleotide suppressed in those cells that have a relatively low level of Lin-28-like polypeptides. In certain embodiments, the loop sequence is heterologous to the stem sequence. In particular embodiments, the loop sequence interacts with a Lin-28-like polypeptide (e.g., Lin-28, Lin-28B). In certain embodiments, the loop has the nucleotide sequence set forth in SEQ ID NO: 102. In some embodiments, the target polynucleotide of the miRNA comprises a disease-causing gene, such as an oncogene.

As one of ordinary skill in the art would appreciate, a presently disclosed pharmaceutical composition is formulated to be compatible with its intended route of administration. Solutions or suspensions used for parenteral (e.g., intravenous), intramuscular, intradermal, or subcutaneous application can include the following components: a sterile diluent such as water for injection, saline solution, fixed oils, polyethylene glycols, glycerine, propylene glycol or other synthetic solvents; antibacterial agents, such as benzyl alcohol or methyl parabens; antioxidants, such as ascorbic acid or sodium bisulfate; chelating agents, such as ethylenediaminetetraacetic acid; buffers, such as acetates, citrates or phosphates; and agents for the adjustment of tonicity, such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use typically include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL™ (BASF, Parsippany, N.J.) or phosphate buffered saline (PBS). The composition should be sterile and should be fluid to the extent that easy syringability exists. In some embodiments, the pharmaceutical compositions are stable under the conditions of manufacture and storage and should be preserved against the contaminating action of microorganisms, such as bacteria and fungi. In general, the relevant carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In some embodiments, isotonic agents, for example, sugars, polyalcohols, such as manitol or sorbitol, or sodium chloride are included in the formulation. Prolonged absorption of the injectable formulation can be brought about by including in the formulation an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. In certain embodiments, solutions for injection are free of endotoxin. Generally, dispersions are prepared by incorporating the active compound into a sterile vehicle which contains a basic dispersion medium and the required other ingredients from those enumerated above. In those embodiments in which sterile powders are used for the preparation of sterile injectable solutions, the solutions can be prepared by vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions also can be prepared using a fluid carrier for use as a mouthwash. Pharmaceutically compatible binding agents, and/or adjuvant materials can be included as part of the composition. The tablets, pills, capsules, troches, and the like can contain any of the following ingredients, or compounds of a similar nature: a binder, such as microcrystalline cellulose, gum tragacanth or gelatin; an excipient, such as starch or lactose, a disintegrating agent, such as alginic acid, Primogel, or corn starch; a lubricant, such as magnesium stearate or Sterotes; a glidant, such as colloidal silicon dioxide; a sweetening agent, such as sucrose or saccharin; or a flavoring agent, such as peppermint, methyl salicylate, or orange flavoring. Compositions for oral delivery can advantageously incorporate agents to improve stability within the gastrointestinal tract and/or to enhance absorption.

For administration by inhalation, the presently disclosed compositions can be delivered in the form of an aerosol spray from a pressured container or dispenser which contains a suitable propellant, e.g., a gas such as carbon dioxide, or a nebulizer. Liquid aerosols, dry powders, and the like, also can be used.

Systemic administration of the presently disclosed compositions also can be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds also can be prepared in the form of suppositories (e.g., with conventional suppository bases such as cocoa butter and other glycerides) or retention enemas for rectal delivery.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical or cosmetic carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of individuals. Guidance regarding dosing is provided elsewhere herein.

Depending on the route of administration, the agent may be coated in a material to protect it from the action of enzymes, acids and other natural conditions which may inactivate the agent. For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

To administer an agent by other than parenteral administration, it may be necessary to coat the agent with, or co-administer the agent with, a material to prevent its inactivation. Enzyme inhibitors include pancreatic trypsin inhibitor, diisopropylfluorophosphate (DEP) and trasylol.

When administering a polynucleotide, the polynucleotide can be injected directly as naked DNA or RNA, by infection using defective or attenuated retrovirals or other viral vectors, or can be coated with lipids or cell-surface receptors or transfecting agents, encapsulated in liposomes, microparticles, or microcapsules, or by administering them in linkage to a peptide which is known to enter the nucleus, by administering it in linkage to a ligand subject to receptor-mediated endocytosis (see, e.g., Wu and Wu (1987) J. Biol. Chem. 262:4429-4432) (which can be used to target cell types specifically expressing the receptors) and so on. In another embodiment, polynucleotide-ligand complexes can be formed in which the ligand comprises a fusogenic viral peptide to disrupt endosomes, allowing the polynucleotide to avoid lysosomal degradation. In yet another embodiment, the polynucleotide can be targeted in vivo for cell specific uptake and expression, by targeting a specific receptor. Alternatively, the polynucleotide can be introduced intracellularly and incorporated within host cell DNA for expression, by homologous recombination (Koller and Smithies (1989) Proc. Natl. Acad. Sci. USA 86:8932-8935; Zijistra et al. (1989) Nature 342:435-438).

### III. Methods of Use

The presently disclosed subject matter provides methods for inhibiting the activity of a Lin-28-like polypeptide, methods for differentiating a cell or inhibiting the growth of a cell, methods for selectively suppressing the expression of a target polynucleotide in a population of cells, methods for enriching a population of cells for stem cells, and methods for treating subjects having a disease or unwanted condition, such as cancer.

Methods for inhibiting the activity of a Lin-28-like polypeptide comprise contacting a cell with a compound, wherein the compound inhibits the Lin-28-like polypeptide-mediated inhibition of at least one of regulated primary microRNA transcript processing by Drosha and regulated precursor microRNA processing by Dicer, thereby inhibiting the activity of the Lin-28-like polypeptide.

As described elsewhere herein, a “compound” can refer to a small molecule, a polynucleotide, or other naturally occurring or synthetically derived molecule that is capable of inhibiting the activity of a Lin-28-like polypeptide. The compound may bind directly to the Lin-28-like polypeptide, inhibiting its activity, or it might indirectly inhibit the activity of the Lin-28-like polypeptide by binding to and regulating the activity of a biological molecule that regulates the activity of the Lin-28-like polypeptide. In some embodiments, the compound inhibits the interaction between the Lin-28-like polypeptide and at least one of a regulated primary microRNA transcript and a precursor microRNA. In certain embodiments, the Lin-28-like polypeptide comprises Lin-28 or Lin-28-B. In some embodiments, the regulated primary microRNA transcript comprises a primary Let-7 microRNA transcript or the regulated precursor microRNA comprises a precursor Let-7 microRNA, wherein the Let-7 miRNA can include, but is not limited to, Let-7, Let-7a-1, Let-7a-2, Let-7a-2, Let-7a-3, Let-7b, Let-7c, Let-7d, Let-7e, Let-7f-1, Let-7f-2, Let-7g, Let-71, and miR-98.

The activity of a Lin-28-like polypeptide (e.g., inhibition of Drosha processing of regulated primary microRNA transcripts or Dicer processing of precursor microRNAs) can be measured using assays known in the art and described elsewhere herein. In some embodiments, the activity of a Lin-28-like polypeptide is inhibited by a compound by at least 5%, at least 10%, at least 20%, at least 30%, at least 40%, at least 50%, at least 60%, at least 70%, at least 80%, at least 90%, at least 95%, at least 96%, at least 97%, at least 98%, at least 99%, or higher.

In certain embodiments, the compound that inhibits the Lin-28-like polypeptide is a polynucleotide that comprises or encodes a sequence corresponding to the loop region of a regulated primary microRNA transcript that is introduced into the cell. Methods for identifying primary microRNA transcripts that are regulated by Lin-28-like polypeptides have been described elsewhere herein. Variants and active fragments of the loop region sequence can also be used to inhibit the activity of a Lin-28-like polypeptide. In some embodiments, the introduced polynucleotide or expression product thereof can not be processed to form a mature endogenous miRNA. In certain embodiments, the polynucleotide comprises or encodes a sequence corresponding to the loop region of the primary microRNA transcript or precursor microRNA of the microRNA selected from the group consisting of: hsa-miR-487h, hsa-miR-193a, hsa-miR-34c, hsa-mAR-34a, hsa-mi6-18a, hsa-miR-18a, hsa-miR-29b, hsa-miR-888, hsa-miR-63, hsa-miR-638, hsa-miR-373, hsa-miR-199h, hsa-miR-874, hsa-miR-556, hsa-miR-29b, hsa-miR-29a, hsa-miR-18b, hsa-miR-1, hsa-miR-605, hsa-miR-374, hsa-miR-193a, hsa-miR-34b, hsa-miR-26b, hsa-miR-944, hsa-miR-106h, hsa-miR-10fjb, hsa-miR-34b, hsa-miR-58, hsa-miR-27a, hsa-miR-891a, hsa-miR-4871:1, hsa-miR-563, hsa-miR-30e-E9, hsa-miR-488, hsa-miR-195, hsa-miR-143, hsa-miR-125, hsa-miR-98, hsa-Let-7, hsa-Let-7a-1, hsa-Let-7a-2, hsa-Let-7a-2, hsa-Let-7a-3, hsa-Let-7b, hsa-Let-7c, hsa-Let-7d, hsa-Let-7e, hsa-Let-7f-1, hsa-Let-7f-2, hsa-Let-7g, and hsa-Let-71, including any variant or fragment thereof that retains the ability to inhibit the Lin-28-like polypeptide-mediated inhibition of Drosha processing of regulated primary microRNA transcripts. The sequence of these mature microRNAs and corresponding loop regions are known in the art and can be found on the online microRNA database miRBase (microrna.sanger.ac.uk/sequences/; Griffiths-Jones et al. (2008) Nucleic Acids Research 36(Database Issue):D154-D158; Griffiths-Jones et al. (2006) Nucleic Acids Research 34(Database Issue):D140-D144; Griffiths-Jones (2004) Nucleic Acids Research 32(Database Issue):D109-D111).

In particular embodiments, the polynucleotide comprises or encodes a nucleotide sequence having at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or more sequence identity to any one of the sequences set forth in SEQ ID NO: 1, 2, 3, 4, 5, 6, 7, 8, 9, 10, 11, 12, 13, 14, or 102, or an active fragment thereof. In some of these embodiments, the polynucleotide or an expression product thereof can not be processed to form a mature Let-7 miRNA when introduced into or expressed in a cell. In other embodiments, the polynucleotide or expression product thereof can not be processed to form a mature endogenous miRNA. In some embodiments, the polynucleotide comprises or encodes the nucleotide sequence set forth in SEQ ID NO: 102.

In some embodiments, the polynucleotide used to inhibit the activity of a Lin-28-like polypeptide is a single-stranded RNA oligonucleotide. In certain embodiments, the polynucleotide comprises modified nucleic acids (such as those defined elsewhere herein) to impart stability to the polynucleotide. In some of these embodiments, the polynucleotide comprises 2′-O-methylated ribonucleotides.

In particular embodiments, the cell that is contacted with the compound comprises a eukaryotic cell. In some of these embodiments, the cell comprises an animal cell, and in certain embodiments, the cell comprises a human cell.

Studies suggest Lin-28 plays a role in maintaining pluripotency in stem cells (Yu et al. (2007) Science 318:1917-1920) and the Let-7 family of microRNAs have been implicated in the regulation of the expression of key genetic regulators of cellular growth and differentiation, including the oncogenes Ras, Myc, and HMGA2 (Esquela-Kerscher and Slack (2006) Nat Rev Cancer 6:259-269; Mayr et al. (2007) Science 315:1576-1579; Lee and Dutta (2007) Genes Dev 21:1025-1030). Thus, the presently disclosed subject matter provides methods for differentiating a cell or inhibiting the growth of a cell comprising introducing into a cell a compound that inhibits the activity of a Lin-28-like polypeptide. In some of these embodiments, the methods comprise introducing into a cell a polynucleotide comprising or encoding a nucleotide sequence corresponding to the loop region of a regulated primary microRNA transcript (e.g., a Lin-28-like polypeptide-regulated primary microRNA transcript) or a loop region of a regulated precursor microRNA (e.g., a Lin-28-like polypeptide-regulated precursor microRNA). In particular embodiments, the polynucleotide comprises or encodes a nucleotide sequence having at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or more sequence identity to any one of the sequences set forth in SEQ ID NO: 1, 2, 3, 4, 5, 6, 7, 8, 9, 10, 11, 12, 13, 14, or 102, or an active fragment thereof, and wherein the polynucleotide promotes the processing of at least one of a regulated primary microRNA transcript by Drosha and a regulated precursor microRNA by Dicer when introduced into or expressed in a cell. In some of these embodiments, the polynucleotide or an expression product thereof can not be processed to form a mature Let-7 miRNA when introduced into or expressed in a cell. In certain embodiments, the polynucleotide or expression product thereof can not be processed to form a mature endogenous miRNA. In some embodiments, the polynucleotide comprises or encodes the nucleotide sequence set forth in SEQ ID NO: 102.

In some of these embodiments, the introduced polynucleotide inhibits the activity of a Lin-28-like polypeptide when introduced into or expressed in a cell, wherein the activity comprises the inhibition of the processing of at least one of a regulated primary microRNA transcript by Drosha and a regulated precursor microRNA by Dicer. In these embodiments, the cell to which the polynucleotide is being introduced expresses a Lin-28-like polypeptide. In some of these embodiments, the polynucleotide inhibits the interaction between the Lin-28-like polypeptide and at least one of a regulated primary microRNA transcript and a regulated precursor microRNA when introduced into or expressed in a cell.

In some embodiments, the regulated primary microRNA transcript comprises a primary Let-7 microRNA transcript or the regulated precursor microRNA comprises a precursor Let-7 microRNA, wherein the Let-7 microRNA includes, but is not limited to, Let-7, Let-7a-1, Let-7a-2, Let-7a-2, Let-7a-3, Let-7b, Let-7c, Let-7d, Let-7e, Let-7f-1, Let-7f-2, Let-7g, Let-71, and miR-98.

According to the presently disclosed methods, the cell wherein the polynucleotide has been introduced exhibits a more differentiated state or reduced growth in comparison to a control cell. In some embodiments, the control cell comprises the cell prior to the introduction of the polynucleotide. In other embodiments, the control cell comprises a cell of the same type with a similar growth rate or differentiation state as the cell of interest prior to the introduction of the polynucleotide.

Any method known in the art can be used to measure the growth rate of a cell, including, but not limited to, optical density (OD₆₀₀), CO₂ production, O₂ consumption, assays that measure mitochondrial function, such as those utilizing tetrazolium salts (e.g., MTT, XTT), or other colorimetric reagents (e.g., the WST-1 reagent available from Roche), assays that measure or estimate DNA content, including, but not limited to fluoremetric assays such as those utilizing the fluorescent dye Hoechst 33258, assays that measure or estimate protein content, including, but not limited to, the sulforhodamine B (SRB) assay, manual or automated cell counts (with or without the Trypan Blue stain to distinguish live cells), and clonogenic assays with manual or automated colony counts. In some embodiments, the growth rate of a cell is inhibited by a compound by at least 5%, at least 10%, at least 20%, at least 30%, at least 40%, at least 50%, at least 60%, at least 70%, at least 80%, at least 90%, at least 95%, at least 96%, at least 97%, at least 98%, at least 99%, or higher.

Any method known in the art can be used to measure the differentiation state of a particular cell and one of skill in the art would recognize that the particular method will depend on the cell of interest, and can include, for example, measuring the expression of various cell surface markers (using, for example, an ELISA or flow cytometric methods), an enzymatic activity, metabolic function, gene expression, or other biological marker associated with particular stages of differentiation.

Data presented elsewhere herein demonstrate that the Lin-28 and Lin-28B polypeptides inhibit the Drosha-mediated processing of primary microRNA transcripts through the recognition of the loop sequence of the primary microRNA transcript. Heo et al. and Ryka et al. have demonstrated that Lin-28 regulates the processing of particular miRNAs at the level of Dicer as well. Thus, the presently disclosed subject matter provides methods for selectively suppressing a target polynucleotide in a population of cells comprising introducing a polynucleotide into the cells, wherein the polynucleotide or an expression product thereof comprises a stem-loop structure that can be recognized and processed by Drosha or Dicer. The loop of the stem-loop structure comprises a sequence of a loop of a regulated primary microRNA transcript (e.g., a Lin-28-like polypeptide-regulated primary microRNA transcript), a loop of a regulated precursor microRNA (e.g., a Lin-28-like polypeptide-regulated precursor microRNA), or an active variant or fragment thereof. The stem of the stem-loop structure comprises a miRNA. The level of Drosha-mediated or Dicer-mediated processing of the polynucleotide or expression product thereof and therefore, the level of suppression of the polynucleotide(s) targeted by the miRNA is negatively correlated with the expression level of a Lin-28-like polypeptide in the cell. A negative correlation defines a relationship between two variables (e.g., level of suppression of target polynucleotide and expression level of Lin-28-like polypeptide), wherein a change in one variable in one direction results in a change in the second variable in the opposite direction. Thus, the expression of the target polynucleotide is suppressed more efficiently in those cells within the population that express relatively low levels of Lin-28-like polypeptides and less efficiently in those cells with relatively high levels of Lin-28-like polypeptides. In other words, in a population of cells wherein a polynucleotide comprising a stem-loop structure with a Lin-28-like polypeptide-regulated loop sequence and an miRNA has been introduced, those cells with higher expression levels of Lin-28-like polypeptides in comparison to other cells will have a lower level of suppression of target polynucleotides (and, therefore, a higher level of expression of the target polynucleotides) in comparison to other cells within the population. Conversely, those cells within a population comprising or expressing the polynucleotide that have lower expression levels of Lin-28-like polypeptides in comparison to other cells will have a higher level of suppression of target polynucleotides and, therefore, a lower level of expression of the target polynucleotides. The expression of the target polynucleotide is selectively suppressed by the miRNA in those cells that have relatively low levels of Lin-28-like polypeptides because the polynucleotide is able to be processed by Drosha or Dicer.

As used herein, a population of cells can refer to a homogeneous or heterogeneous population of cells. The population of cells may comprise one or more cell lines, one or more primary cell cultures, a tissue comprising one or more types of cells. The population may also comprise cells of the same genotype (e.g., from the same cell line) that differ in the growth rate, differentiation state, pathological state, or environmental conditions.

In some embodiments, the population of cells can comprise stem cells. As used herein, a “stem cell” refers to a cell with the capability of multi-lineage differentiation and self-renewal. Stem cells can comprise any type of cell with these properties, including but not limited to stem cells that are pluripotent, multipotent, oligopotent, and unipotent. As used herein, a “pluripotent stem cell” refers to those cells that can differentiate or can be induced to differentiate into cells of any of the three germ layers (endoderm, mesoderm, or ectoderm). Pluripotent stem cells can include both naturally occurring pluripotent stem cells, such as embryonic stem (ES) cells that are isolated from the inner cell mass of the blastocyst, embryonic germ (EG) cells, and embryonic carcinoma (EC) cells, as well as induced or “reprogrammed” pluripotent stem (iPS) cells. Induced pluripotent stem (iPS) cells can be generated by reprogramming adult cells into pluripotent stem cells through the introduction and expression of particular genes (Yu et al. (2007) Science 318:1917-1920; Takahashi et al. (2007) Cell 131:861-872; Nakagawa et al. (2008) Nature Biotech 26:101-106; Kim et al. (2008) Nature 454:646-650). A “multipotent stem cell” or “oligopotent stem cell” refers to those stem cells that can differentiate into multiple cell types of a limited number. For example, hematopoietic stem cells can give rise to various types of blood cells. A “unipotent stem cell” is a stem cell that can only differentiate into one cell type, but still retains self-renewal capabilities.

In particular embodiments, the loop of the stem-loop structure has a nucleotide sequence having at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or more sequence identity to any one of the sequences set forth in SEQ ID NO: 1, 2, 3, 4, 5, 6, 7, 8, 9, 10, 11, 12, 13, 14, or 102, or an active fragment thereof. In some of these embodiments, active fragments and variants of the disclosed sequences are those that are capable of interacting (directly or indirectly) with a Lin-28-like polypeptide such that the Lin-28-like polypeptide inhibits the processing of the polynucleotide comprising the fragment or variant of the disclosed sequence. In some embodiments, the stem sequence is heterologous to the loop sequence. In particular embodiments, the polynucleotide can not be processed to form a mature Let-7 miRNA. In certain embodiments, the loop has the nucleotide sequence set forth in SEQ ID NO: 102.

Lin28 is expressed at the early stages of embryonic development, including undifferentiated ES cells, and embryonic carcinoma cells (Polesskaya et al. (2007) Genes Dev 21:1125-1138; Richards et al. (2004) Stem Cells 22:51-64; Yang and Moss (2003) Gene Expr. Patterns 3:719-726). Lin28B is also highly expressed in poorly differented hepatocellular carcinomas and in cell lines derived therefrom, including Huh7, HepG2, and Hep3B (Guo et al. (2006) Gene 384:51-61). Yu et al. demonstrated that the introduction of Lin28, along with Oct4, Sox2, and Nanog are sufficient to reprogram human somatic fibroblasts into pluripotent stem cells, suggesting an important role for Lin28 in the maintenance of pluripotency. Thus, the presently disclosed subject matter provides methods of enriching for stem cells in populations of cells comprising the same. The methods comprise introducing a polynucleotide into the cells of the population, wherein the polynucleotide or expression product thereof comprises a stem-loop structure that can be recognized and processed by Drosha or Dicer and wherein the loop of the stem-loop structure has a nucleotide sequence of a loop of a regulated primary microRNA transcript or a regulated precursor microRNA transcript, or an active variant or fragment thereof. In particular embodiments, the loop sequence is from a loop of a Lin-28-like polypeptide-regulated primary microRNA transcript, a Lin-28-like polypeptide-regulated precursor microRNA or an active variant or fragment thereof. In some of these embodiments, the loop has a nucleotide sequence having at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99% or more sequence identity to any one of the sequences set forth in SEQ ID NO: 1, 2, 3, 4, 5, 6, 7, 8, 9, 10, 11, 12, 13, 14, or 102, or an active fragment thereof. The stem of the stem-loop structures comprises a microRNA, wherein the suppression of a target polynucleotide of the microRNA leads to a suppression of cellular growth or viability. In some embodiments, the loop sequence is heterologous to the stem sequence. In certain embodiments, the loop sequence can interact with a Lin-28-like polypeptide. In certain embodiments, the loop has the nucleotide sequence set forth in SEQ ID NO: 102.

The processing of the polynucleotide or expression product thereof by Drosha or Dicer is inhibited by a Lin-28-like polypeptide. Thus, the miRNA of the stem-loop structure will be selectively processed by Drosha or Dicer based on the level of expression of Lin-28-like polypeptides. Thus, the target polynucleotide will be suppressed in those cells that have a relatively low level of Lin-28-like polypeptides. The level of suppression of a target polynucleotide by the miRNA within the stem-loop structure is negatively correlated with the expression level of a Lin-28-like polypeptide in the cell into which the polynucleotide has been introduced or expressed. Due to the high level of Lin-28 expression in stem cell populations (ES and EC cells), this method can be used to selectively enrich a population of cells for stem cells. In some embodiments, the population of cells comprise pluripotent stem cells, and in particular embodiments, induced pluripotent stem cells, and these types of stem cells can be enriched using the presently disclosed methods.

The presently disclosed methods for the enrichment and isolation of pluripotent stem cells provide an advantage over the methods presently in use, wherein adult cells are reprogrammed into pluripotent stem cells through the introduction of particular genes, many of which are oncogenic (e.g., Oct4, c-Myc).

Target polynucleotides of miRNAs whose suppression would lead to a suppression of cellular growth or viability include those genes that are essential for or contribute to cellular survival or growth. Non-limiting examples of such genes that contribute to or are essential for cellular survival or growth include those listed in Schlabach et al. (2008) Science 319(5863):620-624. In some embodiments, the target polynucleotide of the miRNA is a polo-like kinase-1 (PLK1) gene, an origin recognition complex-2 (ORC2) gene, or a cell-divisional cycle-2 (CDC2) gene.

In some embodiments, the introduction of the presently disclosed polynucleotides comprising microRNAs leads to the suppression of target polynucleotides of the microRNA, leading to the suppression of cellular growth or viability, wherein the growth rate of a cell or the survival of cells within a population of cells is inhibited by at least 5%, at least 10%, at least 20%, at least 30%, at least 40%, at least 50%, at least 60%, at least 70%, at least 80%, at least 90%, at least 95%, at least 96%, at least 97%, at least 98%, at least 99%, or higher. Assays to measure cellular growth or viability are known in the art and described elsewhere herein.

The presently disclosed subject matter also provides for methods for treating a disease or unwanted condition in a subject comprising administering to the subject a compound, wherein the compound inhibits the activity of a Lin-28-like polypeptide, wherein the activity comprises the inhibition of the processing of a regulated primary microRNA transcript by Drosha. In other embodiments, the subject in need thereof can be administered a polynucleotide, wherein the polynucleotide or expression product thereof comprises a stem-loop structure with a loop sequence from a regulated primary microRNA transcript, a precursor microRNA, or an active variant or fragment thereof, and a microRNA, wherein a target polynucleotide of the microRNA comprises a disease-causing gene and the polynucleotide or expression product thereof can be recognized by Dicer or Drosha and wherein the Drosha-mediated processing or Dicer-mediated processing is inhibited by a Lin-28-like polypeptide.

As used herein, the terms “treatment” or “prevention” refer to obtaining a desired pharmacologic and/or physiologic effect. The effect may be prophylactic in terms of completely or partially preventing a particular infection or disease or sign or symptom thereof and/or may be therapeutic in terms of a partial or complete cure of an infection or disease and/or adverse effect attributable to the infection or the disease. Accordingly, the method “prevents” (i.e., delays or inhibits) and/or “reduces” (i.e., decreases, slows, or ameliorates) the detrimental effects of a disease or disorder in the subject receiving the compositions of the invention. “Treatment” can also mean prolonging survival as compared to expected survival if not receiving treatment. Survival can be prolonged by at least about one month, at least about 2 months, at least about 6, at least about 9 months, at least about a year, at least about 2 years, at least about 5 years, at least about 10 years, at least about 20 years, at least about 30 years or longer following treatment with the compound described herein.

By “subject” or “patient” is intended an animal, including a mammal, such as a human, and including, but by no means limited to, domestic animals, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, cats, etc., avian species, such as chickens, turkeys, songbirds, etc., i.e., for veterinary medical use. In particular embodiments, the subject is a human.

The subject to be treated can have any disease or unwanted condition. In some embodiments, the disease is one that would benefit from cell growth inhibition or promotion of cellular differentiation. In some embodiments, the subject to be treated has a cancer. The terms “cancer” or “cancerous” refer to or describe the pathological condition that is typically characterized by unregulated cell growth. The term “cancer” encompasses all types of cancers, including, but not limited to, all forms of carcinomas, melanomas, sarcomas, lymphomas and leukemias, including without limitation, bladder carcinoma, brain tumors, breast cancer, cervical cancer, colorectal cancer, esophageal cancer, endometrial cancer, hepatocellular carcinoma, laryngeal cancer, lung cancer, osteosarcoma, ovarian cancer, pancreatic cancer, prostate cancer, renal carcinoma and thyroid cancer.

In some embodiments, the compound binds to and inhibits the activity of the Lin-28-like polypeptide. In other embodiments, the compound binds to and regulates the activity of a biological molecule that regulates the activity of the Lin-28-like polypeptide. In some embodiments, the compound inhibits the interaction between the Lin-28-like polypeptide and at least one of a regulated primary microRNA transcript and a regulated precursor microRNA, as defined elsewhere herein. In particular embodiments, the Lin-28-like polypeptide comprises Lin-28 or Lin-28B.

In some embodiments of the presently disclosed methods, the regulated primary microRNA transcript comprises a primary Let-7 microRNA transcript or the regulated precursor microRNA comprises a precursor Let-7 microRNA, wherein the Let-7 microRNA can include, but is not limited to, Let-7, Let-7a-1, Let-7a-2, Let-7a-3, Let-7b, Let-7c, Let-7d, Let-7e, Let-7f-1, Let-7f-2, Let-7g, Let-71, and miR-98.

In certain embodiments, the compound that inhibits the Lin-28-like polypeptide is a polynucleotide that when introduced into or expressed in a cell inhibits the activity of the Lin-28-like polypeptide. In some of these embodiments, the polynucleotide comprises or encodes a sequence corresponding to the loop region of a microRNA of a regulated primary microRNA transcript. Variants and active fragments of the loop region sequence can also be used to inhibit the activity of a Lin-28-like polypeptide. In certain embodiments, the introduced polynucleotide or an expression product thereof can not be processed to form a mature endogenous miRNA. In some embodiments, the polynucleotide comprises or encodes a sequence corresponding to the loop region of a microRNA selected from the group consisting of: hsa-miR-487h, hsa-miR-193a, hsa-miR-34c, hsa-mAR-34a, hsa-mi6-18a, hsa-miR-18a, hsa-miR-29b, hsa-miR-888, hsa-miR-63, hsa-miR-638, hsa-miR-373, hsa-miR-199h, hsa-miR-874, hsa-miR-556, hsa-miR-29b, hsa-miR-29a, hsa-miR-18b, hsa-miR-1, hsa-miR-605, hsa-miR-374, hsa-miR-193a, hsa-miR-34b, hsa-miR-26b, hsa-miR-944, hsa-miR-106h, hsa-miR-10fjb, hsa-miR-34b, hsa-miR-58, hsa-miR-27a, hsa-miR-891a, hsa-miR-4871:1, hsa-miR-563, hsa-miR-30e-E9, hsa-miR-488, hsa-miR-195, hsa-miR-143, hsa-miR-125, hsa-miR-98, hsa-Let-7, hsa-Let-7a-1, hsa-Let-7a-2, hsa-Let-7a-2, hsa-Let-7a-3, hsa-Let-7b, hsa-Let-7c, hsa-Let-7d, hsa-Let-7e, hsa-Let-7f-1, hsa-Let-7f-2, hsa-Let-7g, and hsa-Let-71, including any variant or fragment thereof that retains the ability to inhibit the Lin-28-like polypeptide-mediated inhibition of at least one of Drosha processing of regulated primary microRNA transcripts and Dicer processing of regulated precursor microRNAs. The sequence of these mature microRNAs and corresponding loop regions are known in the art and can be found on the online microRNA database miRBase (microrna.sanger.ac.uk/sequences/; Griffiths-Jones et al. (2008) Nucleic Acids Research 36(Database Issue):D154-D158; Griffiths-Jones et al. (2006) Nucleic Acids Research 34(Database Issue):D140-D144; Griffiths-Jones (2004) Nucleic Acids Research 32(Database Issue):D109-D111).

In particular embodiments, the polynucleotide comprises or encodes a nucleotide sequence having at least 40%, at least 45%, at least 50%, at least 55%, at least 60%, at least 65%, at least 70%, at least 80%, at least 90%, at least 95%, at least 96%, at least 97%, at least 98%, at least 99% or more sequence identity to any one of the sequences set forth in SEQ ID NO:1, 2, 3, 4, 5, 6, 7, 8, 9, 10, 11, 12, 13, 14, or 102 or an active fragment thereof. In some embodiments, the polynucleotide or an expression product thereof can not be processed to form a mature Let-7 miRNA when introduced into or expressed in a cell. In other embodiments, the polynucleotide or expression product thereof can not be processed to form a mature endogenous miRNA. In certain embodiments, the polynucleotide used to inhibit the activity of a Lin-28-like polypeptide is a single-stranded RNA oligonucleotide. In certain embodiments, the polynucleotide comprises or encodes the nucleotide sequence set forth in SEQ ID NO: 102.

In some embodiments, the isolated polynucleotide useful for the treatment of diseases comprises or encodes a stem-loop structure that can be recognized and processed by Dicer or Drosha, wherein the loop of the stem-loop structure has the loop sequence of a regulated primary microRNA transcript, a regulated precursor microRNA, or an active variant or fragment thereof. In particular embodiments, the loop sequence is from a loop of a Lin-28-like polypeptide-regulated primary microRNA transcript, a Lin-28-like polypeptide-regulated precursor microRNA or an active variant or fragment thereof. In some of these embodiments, the loop has a nucleotide sequence having at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99% or more sequence identity to any one of the sequences set forth in SEQ ID NO: 1, 2, 3, 4, 5, 6, 7, 8, 9, 10, 11, 12, 13, 14, or 102, or an active fragment thereof. The stem of the stem-loop structures comprise a microRNA, wherein a target polynucleotide of the miRNA is a disease-causing gene. The processing of the polynucleotide or expression product thereof by at least one of Drosha and Dicer is inhibited by a Lin-28-like polypeptide. In some embodiments, the loop has a sequence that is heterologous to the sequence of the stem of the stem-loop structure. In certain embodiments, the loop sequence can interact with a Lin-28-like polypeptide. In some embodiments, the loop has the nucleotide sequence set forth in SEQ ID NO: 102.

In these embodiments, a target polynucleotide of the miRNA is a disease-causing gene, such as an oncogene. In those embodiments wherein a target polynucleotide is an oncogene, the methods are useful for the treatment of a cancer. Thus, the methods provide for the selective suppression of target polynucleotides (e.g., oncogenes) in those tissues that express relatively low levels of Lin-28-like polypeptides. Lin-28 is expressed at high levels in stem cells, wherein it plays a role in maintaining pluripotency (Yu et al. (2007) Science 318:1917-1920). Therefore, a polynucleotide having a stem-loop structure comprising a loop sequence from a Lin-28-like polypeptide-regulated primary miRNA transcript or Lin-28-like polypeptide-regulated precursor miRNA (e.g., Let-7) would not be efficiently processed by at least one of Drosha and Dicer and the target polynucleotide would not be effectively suppressed in those tissues that express relatively high levels of Lin-28, such as pluripotent stem cells. Selectively suppressing disease-causing genes (e.g., oncogenes) in those cells with relatively low levels of Lin-28 will reduce unwanted side effects of disease-causing gene-targeting miRNAs, improving their therapeutic index (described in more detail hereinbelow). These methods will be most effective in those subjects having a disease, wherein the diseased cells express Lin-28-like polypeptides at a lower level than normal cells (e.g., stem cells).

In certain embodiments wherein a pharmaceutical composition comprising a polynucleotide is administered to a subject, the polynucleotide comprises modified nucleic acids (such as those defined elsewhere herein) to impart stability to the polynucleotide. In some of these embodiments, the polynucleotide comprises 2′-O-methylated ribonucleotides.

The presently disclosed subject matter also provides for the use of the presently disclosed compounds and polynucleotides for the manufacture of a medicament for the treatment of various diseases (e.g., cancer). The polynucleotide can be one that promotes at least one of Drosha-mediated processing of regulated primary microRNA transcripts and Dicer-mediated processing of regulated precursor microRNAs. In other embodiments, the polynucleotide can be one that comprises a stem-loop structure and can be recognized and processed by Dicer or Drosha, wherein the loop of said stem-loop structure has a nucleotide sequence from the loop of a regulated primary miRNA transcript or precursor miRNA or an active fragment thereof, wherein the stem of said stem-loop structure comprises a miRNA that has a target polynucleotide that is a disease-causing gene, wherein the loop has a sequence that is heterologous to the sequence of said stem of said stem-loop structure, and wherein the processing of said polynucleotide or expression product thereof by at least one of Dicer and Drosha is inhibited by a Lin-28-like polypeptide.

The ability of a compound to effectively treat a cancer may be measured by many parameters, for instance, a reduced tumor size or tumor load, reduced development or growth of primary or secondary tumors, or reduced severity of secondary effects of the disease.

It will be understood by one of skill in the art that the treatment modalities described herein may be used alone or in conjunction with other therapeutic modalities (i.e., as adjuvant therapy), including, but not limited to, surgical therapy, radiotherapy, chemotherapy. The Lin-28 inhibitory compound or polynucleotide comprising or encoding a stem-loop structure comprising a microRNA can be administered to the subject prior to, following, or in some cases, simultaneously with other therapeutic modalities.

Delivery of a therapeutically effective amount of a compound that inhibits the activity of a Lin-28-like polypeptide can be obtained via administration of a pharmaceutical composition comprising a therapeutically effective dose of this agent. By “therapeutically effective amount” or “dose” is meant the concentration of a compound that is sufficient to elicit the desired therapeutic effect.

As used herein, “effective amount” is an amount sufficient to effect beneficial or desired clinical or biochemical results. An effective amount can be administered one or more times.

The effective amount of the compound will vary according to the weight, sex, age, and medical history of the subject. Other factors which influence the effective amount can include, but are not limited to, the severity of the subject's condition, the disorder being treated, the stability of the polynucleotide or compound, and, if desired, the adjuvant therapeutic agent being administered along with the compound. Methods to determine efficacy and dosage are known to those skilled in the art. See, for example, Isselbacher et al. (1996) Harrison's Principles of Internal Medicine 13 ed., 1814-1882, herein incorporated by reference.

Toxicity and therapeutic efficacy of such compounds can be determined by standard pharmaceutical methods in cell cultures or experimental animals, e.g., for determining the LD₅₀ (the dose lethal to 50% of the population) and the ED₅₀ (the dose therapeutically effective in 50% of the population).

The dose ratio between toxic and therapeutic effects is the therapeutic index and it can be expressed as the ratio LD₅₀/ED₅₀. Compounds which exhibit high therapeutic indices are preferred. While compounds that exhibit toxic side effects can be used, care should be taken to design a delivery system that targets such compounds to the site of affected tissue to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used in the presently disclosed methods, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

A therapeutically effective amount of a pharmaceutical formulation typically ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The pharmaceutical formulation can be administered at various intervals and over different periods of time as required, e.g., multiple times per day, daily, every other day, once a week for between about 1 to 10 weeks, between 2 to 8 weeks, between about 3 to 7 weeks, about 4, 5, or 6 weeks, and the like. The skilled artisan will appreciate that certain factors can influence the dosage and timing required to effectively treat a subject, including but not limited to the severity of the disease, disorder, or unwanted condition, previous treatments, the general health and/or age of the subject, and other diseases or unwanted conditions present. Generally, treatment of a subject can include a single treatment or, in many cases, can include a series of treatments. Further, treatment of a subject can include a single cosmetic application or, in some embodiments, can include a series of cosmetic applications.

Exemplary doses include milligram or microgram amounts of the inventive compound per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram.) For local administration (e.g., intranasal), smaller doses can be used. It is furthermore understood that appropriate doses of a compound depend upon its potency and can optionally be tailored to the particular recipient, for example, through administration of increasing doses until a preselected desired response is achieved. It is understood that the specific dose level for any particular animal subject can depend on a variety of factors including the activity of the specific compound employed, the age, body weight, general health, gender, and diet of the subject, the time of administration, the route of administration, the rate of excretion, any drug combination, and the degree of expression or activity to be modulated.

One of ordinary skill in the art upon review of the presently disclosed subject matter would appreciate that the presently disclosed compounds, including pharmaceutical compositions thereof, can be administered directly to a cell, a cell culture, a cell culture medium, a tissue, a tissue culture, a tissue culture medium, and the like. When referring to the compounds of the invention, the term “administering,” and derivations thereof, comprises any method that allows for the compound to contact a cell. The presently disclosed compounds can be administered to (or contacted with) a cell or a tissue in vitro or ex vivo. The presently disclosed compounds also can be administered to (or contacted with) a cell or a tissue in vivo by administration to an individual subject, e.g., a patient, for example, by systemic administration (e.g., intravenous, intraperitoneal, intramuscular, subdermal, or intracranial administration) or topical application, as described elsewhere herein.

### IV. Hybridization Techniques

In hybridization techniques, all or part of a known polynucleotide is used as a probe that selectively hybridizes to other corresponding polynucleotides present in a population of cloned genomic DNA fragments or cDNA fragments (i.e., genomic or cDNA libraries) from a chosen organism. The hybridization probes may be genomic DNA fragments, cDNA fragments, RNA fragments, or other oligonucleotides, and may be labeled with a detectable group such as ³²P, or any other detectable marker. Thus, for example, probes for hybridization can be made by labeling synthetic oligonucleotides based on any one of the polynucleotides of the invention. Methods for preparation of probes for hybridization and for construction of cDNA and genomic libraries are generally known in the art and are disclosed in Sambrook et al. (1989) Molecular Cloning: A Laboratory Manual (2d ed., Cold Spring Harbor Laboratory Press, Plainview, N.Y.).

For example, the entire sequence of any of the polynucleotides disclosed herein, or one or more portions thereof, may be used as a probe capable of specifically hybridizing to corresponding polynucleotides. To achieve specific hybridization under a variety of conditions, such probes include sequences that are unique among the polynucleotide sequences and are optimally at least about 10 nucleotides in length, and most optimally at least about 20 nucleotides in length. Such probes may be used to amplify corresponding polynucleotides from a chosen organism by PCR. Hybridization techniques include hybridization screening of plated DNA libraries (either plaques or colonies; see, for example, Sambrook et al. (1989) Molecular Cloning: A Laboratory Manual (2d ed., Cold Spring Harbor Laboratory Press, Plainview, N.Y.).

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, optimally less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1× SSC at 55 to 60° C. Exemplary high stringency conditions include hybridization in 50% formamide, 1 M NaCl, 1% SDS at 37° C., and a wash in 0.1×SSC at 60 to 65° C. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

Specificity is typically the function of post-hybridization washes, the critical factors being the ionic strength and temperature of the final wash solution. For DNA-DNA hybrids, the Tₘ can be approximated from the equation of Meinkoth and Wahl (1984) Anal. Biochem. 138:267-284: Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization, and/or wash conditions can be adjusted to hybridize to sequences of the desired identity. For example, if sequences with >90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution), it is optimal to increase the SSC concentration so that a higher temperature can be used. An extensive guide to the hybridization of nucleic acids is found in Tijssen (1993) Laboratory Techniques in Biochemistry and Molecular Biology—Hybridization with Nucleic Acid Probes, Part I, Chapter 2 (Elsevier, N.Y.); and Ausubel et al., eds. (1995) Current Protocols in Molecular Biology, Chapter 2 (Greene Publishing and Wiley-Interscience, New York). See Sambrook et al. (1989) Molecular Cloning: A Laboratory Manual (2d ed., Cold Spring Harbor Laboratory Press, Plainview, N.Y.).

### V. Sequence Identity

The following terms are used to describe the sequence relationships between two or more polynucleotides or polypeptides: (a) “reference sequence”, (b) “comparison window”, (c) “sequence identity”, and, (d) “percentage of sequence identity.”

(a) As used herein, “reference sequence” is a defined sequence used as a basis for sequence comparison. A reference sequence may be a subset or the entirety of a specified sequence; for example, as a segment of a transcript, or the complete mRNA transcript.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two polynucleotides. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Thus, the determination of percent sequence identity between any two sequences can be accomplished using a mathematical algorithm. Non-limiting examples of such mathematical algorithms are the algorithm of Myers and Miller (1988) CABIOS 4:11-17; the local alignment algorithm of Smith et al. (1981) Adv. Appl. Math. 2:482; the global alignment algorithm of Needleman and Wunsch (1970) J. Mol. Biol. 48:443-453; the search-for-local alignment method of Pearson and Lipman (1988) Proc. Natl. Acad. Sci. 85:2444-2448; the algorithm of Karlin and Altschul (1990) Proc. Natl. Acad. Sci. USA 872264, modified as in Karlin and Altschul (1993) Proc. Natl. Acad. Sci. USA 90:5873-5877.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the GCG Wisconsin Genetics Software Package, Version 10 (available from Accelrys Inc., 9685 Scranton Road, San Diego, Calif., USA). Alignments using these programs can be performed using the default parameters. The CLUSTAL program is well described by Higgins et al. (1988) Gene 73:237-244 (1988); Higgins et al. (1989) CABIOS 5:151-153; Corpet et al. (1988) Nucleic Acids Res. 16:10881-90; Huang et al. (1992) CABIOS 8:155-65; and Pearson et al. (1994) Meth. Mol. Biol. 24:307-331. The ALIGN program is based on the algorithm of Myers and Miller (1988) supra. A PAM120 weight residue table, a gap length penalty of 12, and a gap penalty of 4 can be used with the ALIGN program when comparing amino acid sequences. The BLAST programs of Altschul et al (1990) J. Mol. Biol. 215:403 are based on the algorithm of Karlin and Altschul (1990) supra. BLAST nucleotide searches can be performed with the BLASTN program, score=100, wordlength=12, to obtain nucleotide sequences homologous to a nucleotide sequence encoding a protein of the invention. BLAST protein searches can be performed with the BLASTX program, score=50, wordlength=3, to obtain amino acid sequences homologous to a protein or polypeptide of the invention. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, PSI-BLAST, the default parameters of the respective programs (e.g., BLASTN for nucleotide sequences, BLASTX for proteins) can be used. See www.ncbi.nlm.nih.gov. Alignment may also be performed manually by inspection.

Unless otherwise stated, sequence identity/similarity values provided herein refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by GAP Version 10.

GAP uses the algorithm of Needleman and Wunsch (1970) J. Mol. Biol. 48:443-453, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the GCG Wisconsin Genetics Software Package for protein sequences are 8 and 2, respectively. For nucleotide sequences the default gap creation penalty is 50 while the default gap extension penalty is 3. The gap creation and gap extension penalties can be expressed as an integer selected from the group of integers consisting of from 0 to 200. Thus, for example, the gap creation and gap extension penalties can be 0, 1, 2, 3, 4, 5, 6, 7, 8, 9, 10, 15, 20, 25, 30, 35, 40, 45, 50, 55, 60, 65 or greater.

GAP presents one member of the family of best alignments. There may be many members of this family, but no other member has a better quality. GAP displays four figures of merit for alignments: Quality, Ratio, Identity, and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the GCG Wisconsin Genetics Software Package is BLOSUM62 (see Henikoff and Henikoff (1989) Proc. Natl. Acad. Sci. USA 89:10915).

(c) As used herein, “sequence identity” or “identity” in the context of two polynucleotides or polypeptide sequences makes reference to the residues in the two sequences that are the same when aligned for maximum correspondence over a specified comparison window.

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

It is to be noted that the term “a” or “an” entity refers to one or more of that entity; for example, “a polynucleotide” is understood to represent one or more polynucleotides. As such, the terms “a” (or “an”), “one or more,” and “at least one” can be used interchangeably herein.

Throughout this specification and the claims, the words “comprise,” “comprises,” and “comprising” are used in a non-exclusive sense, except where the context requires otherwise.

As used herein, the term “about,” when referring to a value is meant to encompass variations of, in some embodiments ±50%, in some embodiments ±40%, in some embodiments ±30%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

The following examples are offered by way of illustration and not by way of limitation.

### EXPERIMENTAL

### Example 1

### Lin-28 regulates the processing of Let-7 microRNA

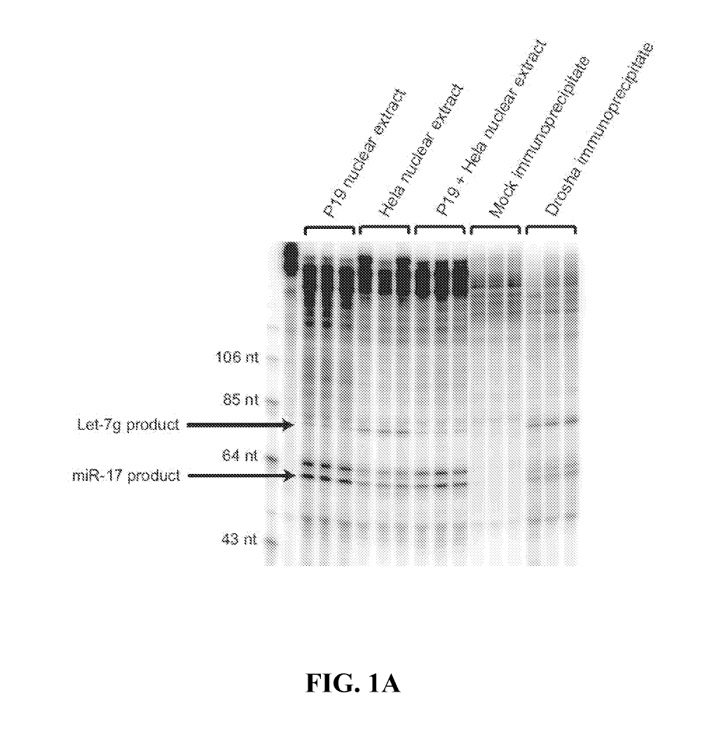

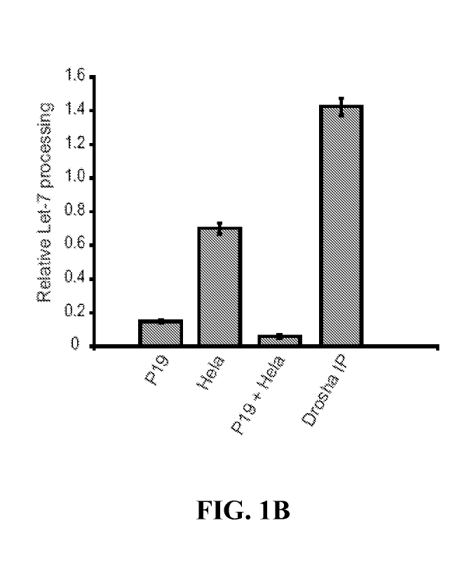

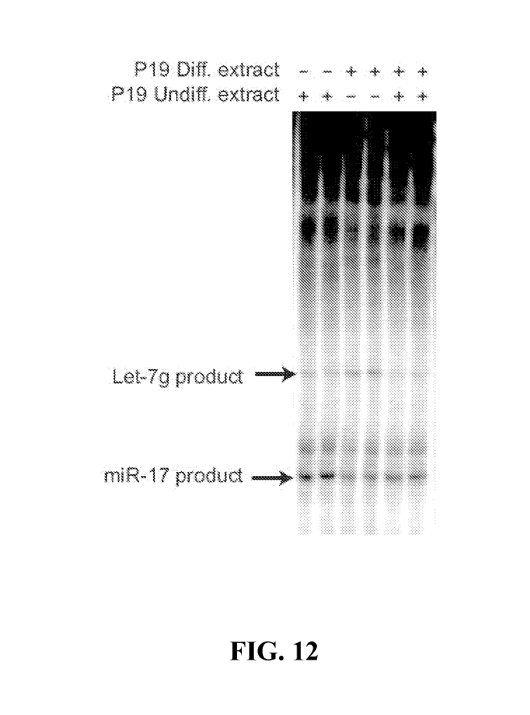

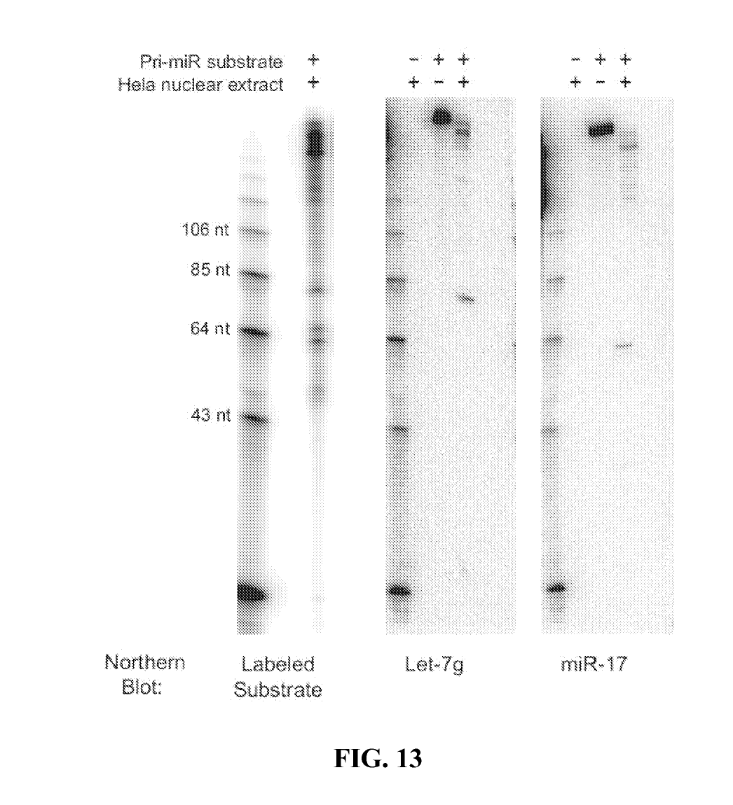

The widespread alterations in miRNA expression in cancer prompted an investigation into the regulatory mechanisms responsible for their production. Initial studies focused on the Let-7 family, where it was previously demonstrated that induction of this miRNA family during mouse embryogenesis occurs at a post-transcriptional stage (Thomson et al. (2006) Genes Dev 20:2202-2207). The presence of a large amount of primary transcript in the absence of precursor and mature species suggested that a block was in place at the Drosha endonuclease processing step. This was tested directly using an established cell-free assay that reports Drosha activity (Lee et al. (2007) Faseb J21:415-426). The processing efficiency of a regulated miRNA, Let-7g, was compared with a miRNA that is readily processed in all known cell types, miR-17. Radiolabeled primary transcripts for these miRNAs were incubated in nuclear extracts prepared from the mouse embryonal carcinoma cell line P19. It has previously been shown that this cell line exhibits regulated processing of Let-7, as it has abundant pri-Let-7 but no detectable mature Let-7 (Thomson et al. (2006) Genes Dev 20:2202-2207). As a control, assays were performed in extracts derived from differentiated P19 cells and Hela cells, two cell types that are competent for Let-7 processing. Nuclear extracts derived from both control cell types were efficient at processing both Let-7g and miR-17, with approximately equal amounts of each precursor product being generated (FIG. 1A and FIG. 12). While several RNA species are generated after the Drosha reaction, the identity of the correct precursor reaction products were confirmed by Northern blot hybridization of unlabeled products (FIG. 13).

While the control nuclear extracts were competent for both Let-7g and miR-17 processing, extracts derived from undifferentiated P19 cells were inefficient at processing Let-7g (FIG. 1A). The ratio of Let-7g product to miR-17 product was calculated to generate a Let-7 processing efficiency. Undifferentiated P19 cells were ˜5 fold less efficient at processing Let-7g compared to miR-17 (FIG. 1B).

This data demonstrates that Drosha processing of Let-7 is less efficient in embryonic cells, but it does not discriminate between an activator present in the Hela extract or an inhibitor present in the P19 extract. Therefore, the same assay was performed in a 1:1 mixed extract of P19 and Hela cells. The mixed extract assay yielded product ratios similar to the P19 extract alone, indicating that the regulatory factor is dominant in P19 cells and therefore an inhibitor of Let-7 processing (FIG. 1A). To rule out the possibility that the regulatory event was due to the modification of Drosha itself, the Drosha protein was immunoprecipitated from undifferentiated P19 nuclear extracts. This purified protein was no longer subject to regulated processing as it was fully competent for Let-7 processing (FIG. 1A).

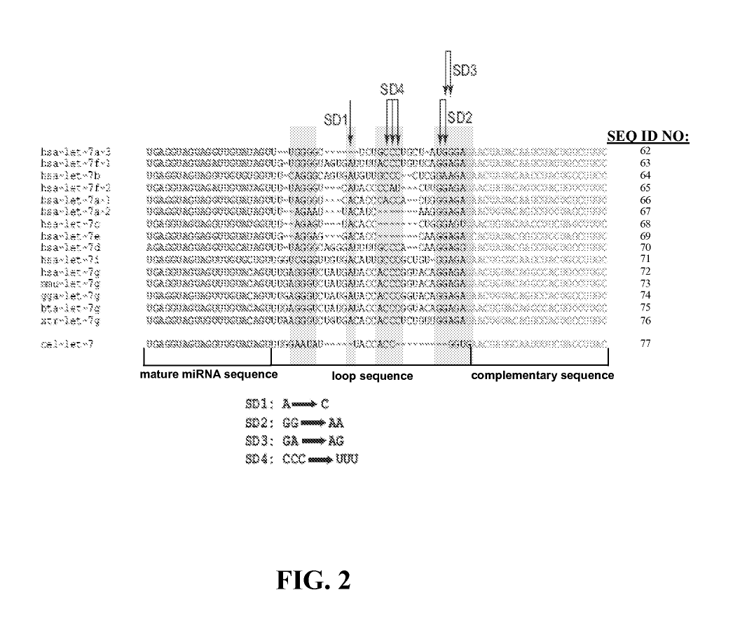

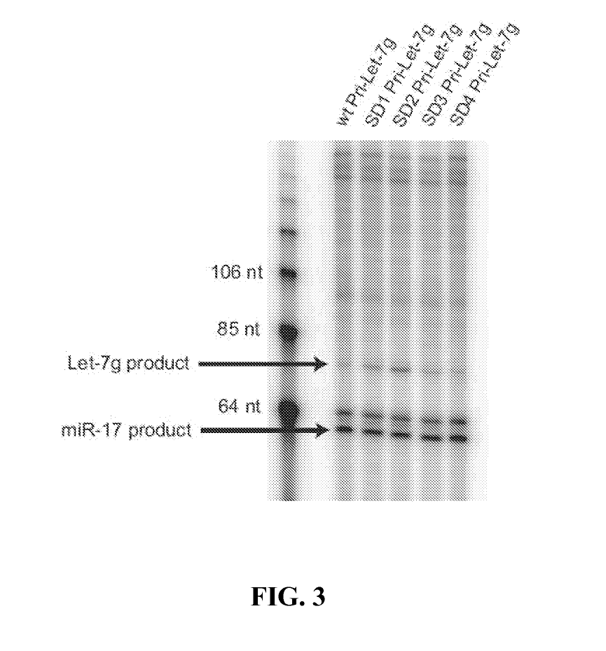

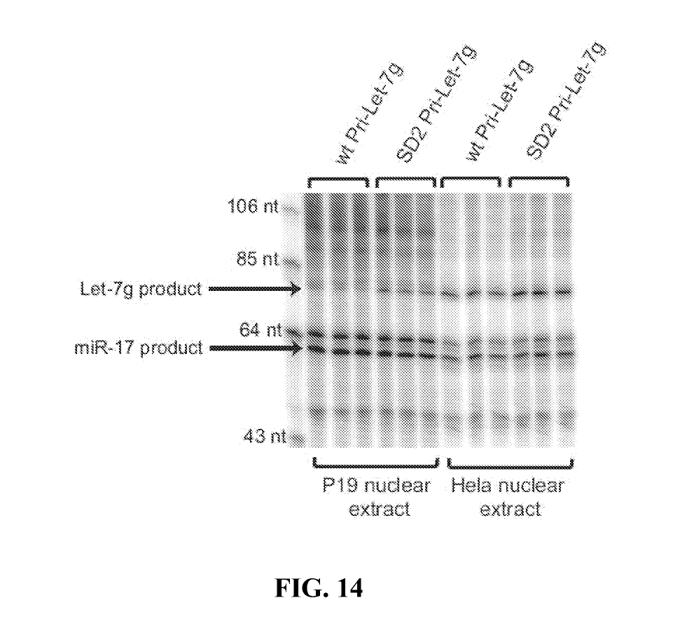

Since Drosha protein that had been immunopurified had lost the regulatory factor, the most logical conclusion of the data was that a Drosha inhibitor, present in the P19 extract, was interacting directly with the Let-7 primary transcript. Alignment of the stem-loop precursors for the Let-7 family revealed highly conserved nucleotides within the loop region (FIG. 2). Several approaches were undertaken to test whether the loop sequences of Let-7 were essential for regulated Drosha processing. The first strategy was to build chimeric Let-7g primary transcripts that contained loop regions from other, unregulated miRNAs. Weak processing was detected in P19 cells, though it was unclear if these altered stem-loops were interacting properly with the processing machinery (data not shown). As an alternative, site-directed mutagenesis was employed to alter conserved residues in the loop (see FIG. 2 for sites). Importantly, these mutations did not affect folding of the stem-loop structure based on computational folding algorithms (mFOLD). Pri-Let-7g transcript containing the SD2 mutation was partially released from the Drosha processing block as evidenced by a 2-fold increase in cleavage in P19 nuclear extracts (FIG. 3 and FIG. 14).

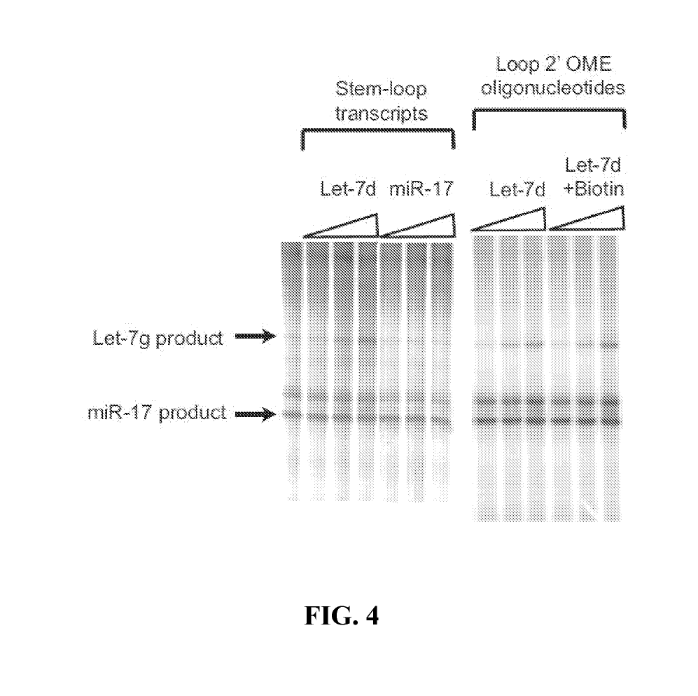

While this data suggests the requirement for these loop nucleotides, it does not formally prove that the Drosha Inhibitor interacts with the loop. To establish this, competitor RNAs based on Let-7 stem loop sequences were designed. If the Drosha inhibitor was binding to the loop region, these competitor RNAs should divert the inhibitor away from the Let-7 primary transcript, restoring processing in P19 cells. Competitors corresponding to all 11 Let-7 family members (data not shown for miR-98) were tested. Competitors that matched Let-7 stem loop sequences restored Drosha processing of Let-7g without affecting production of miR-17 (data not shown). FIG. 4 illustrates restoration of Let-7g processing in P19 nuclear extracts in the presence of Let-7d stem loop competitor. Competitors based on an unregulated miRNA, miR-17, had no effect on Let-7 processing (FIG. 4). Interestingly, an oligonucleotide corresponding to the loop alone was highly effective, suggesting that a stem loop structure is not essential for interaction with regulatory proteins (FIG. 4).

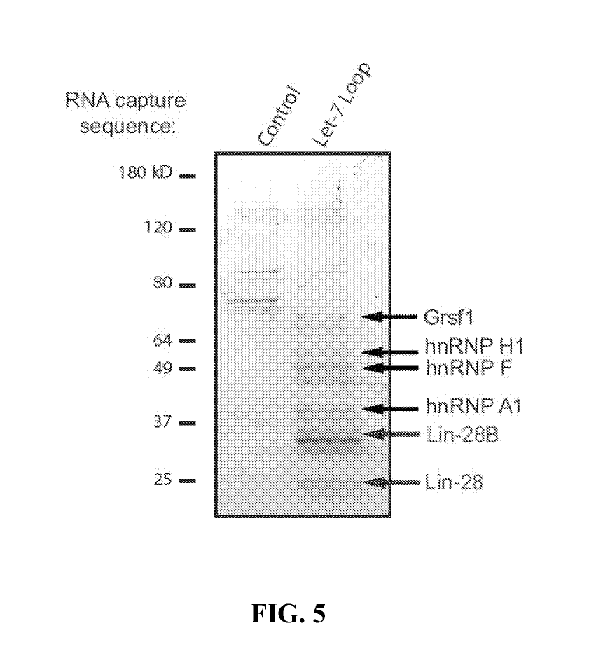

Next, the nature of the Drosha inhibitor was investigated. An affinity purification strategy was designed using the Let-7d loop as a capture reagent. A linear sequence, comprised of loop sequence only, fully 2′-O-methyl modified (for stability), and linked to a biotin moiety, was able to restore Drosha processing of Let-7g when used as a competitor (FIG. 4). This capture probe was bound to streptavidin-agarose and binding proteins were isolated from P19 nuclear extracts. Proteins were identified by peptide mass fingerprinting. A large number of proteins specifically bound to the Let-7 loop probe, as shown in FIG. 5. Many of the proteins belong to the hnRNP family. This family of RNA binding proteins has diverse roles in RNA splicing and has been implicated in the regulation of miR-18a processing (Guil & Caceres (2007) Nat Struct Mol Biol 14:591-596). Interestingly, the RNA binding proteins Lin-28 and Lin-28B were also captured. This protein family was originally identified in C. elegans as a genetic mutant that exhibited abnormal cell lineage (Ambros & Horvitz (1984) Science 226:409-416). Family members are characterized as having a cold shock domain and two zinc-finger domains; a unique domain organization among all known RNA binding proteins (Moss, Lee, and Ambros (1997) Cell 88:637-646).

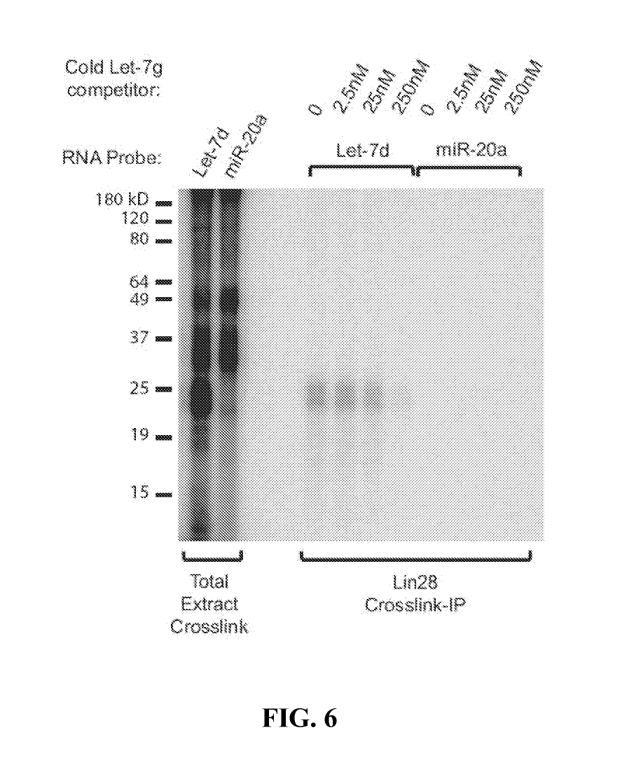

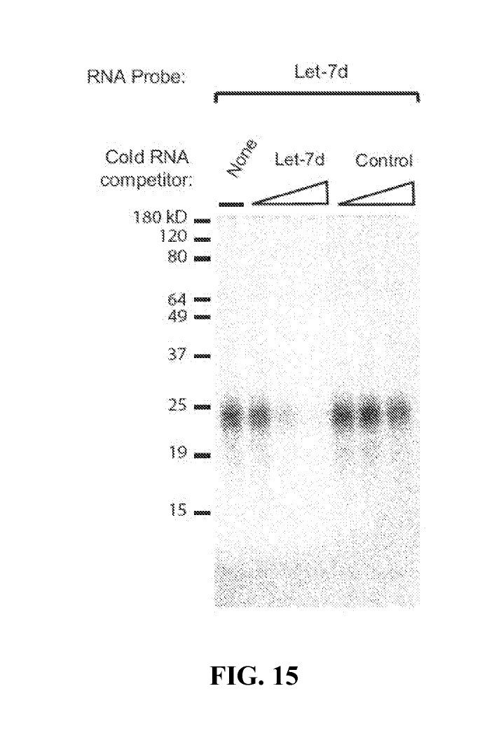

The interaction between Lin-28 and the Let-7 loop was confirmed by UV-crosslink analysis. Radiolabeled Let-7 stem loop RNA specifically crosslinked a protein in P19 nuclear extracts that matched the size of Lin-28 (FIG. 6). The identity of the protein was confirmed by immunoprecipitation of crosslink reactions with an antibody to Lin-28. As evident in FIG. 6, one labeled protein of the correct size was recovered from Let-7 crosslink reactions, but no detectable protein was immunoprecipitated from the control reaction. Importantly, the Let-7/Lin-28 interaction was sensitive to the presence of excess competitor Let-7 stem loop RNA. Control competitors did not disrupt the Lin-28/Let-7 interaction (FIG. 15).

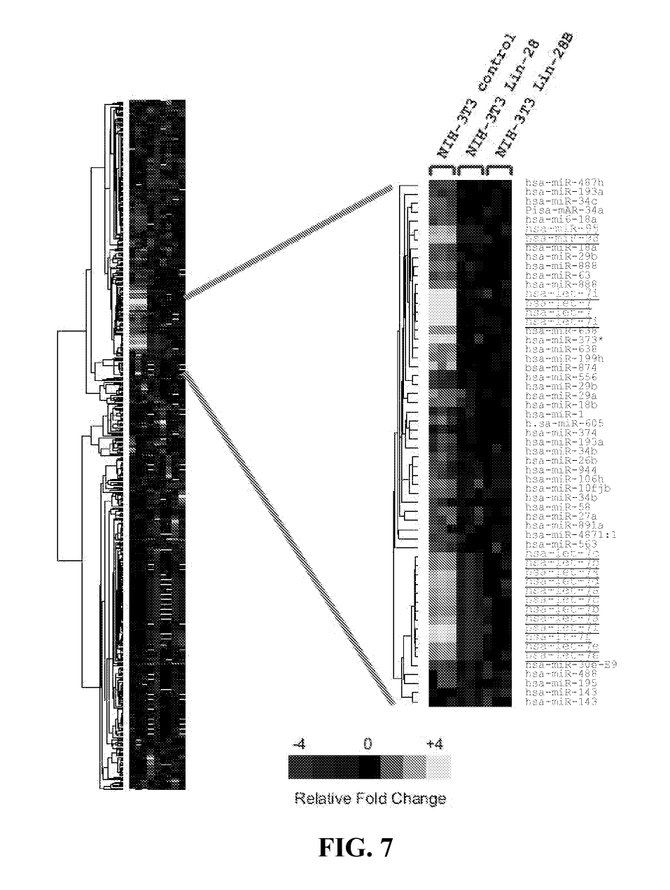

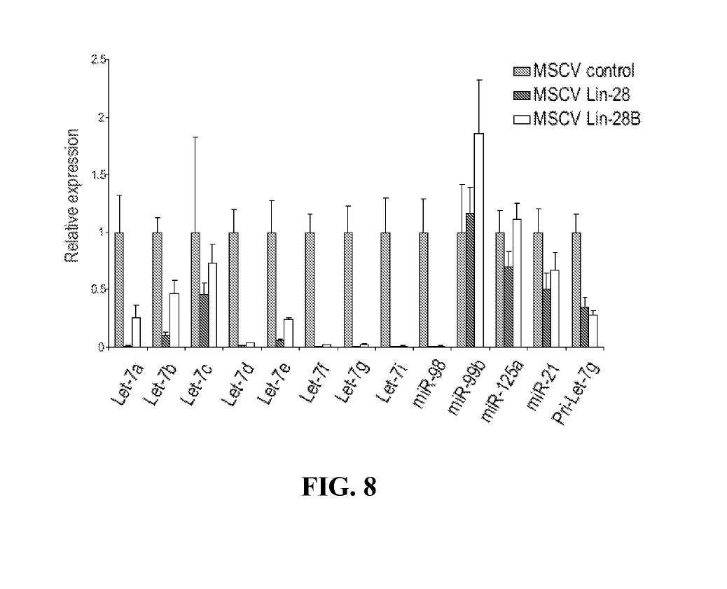

While this data demonstrated interaction of Lin-28 with the Let-7 stem-loop, it did not demonstrate a regulatory role. To test this, Lin-28 and Lin-28B were introduced into NIH-3T3 cells by retroviral transduction and steady state levels of mature miRNAs were measured. Microarray analysis indicated that most miRNAs were unchanged, except for a cluster of miRNAs reduced in cells expressing either Lin-28 or Lin-28B (FIG. 7). As predicted, this cluster included most Let-7 family members. MiRNA expression changes were confirmed by RT-PCR (FIG. 8). This method is more sensitive and provides greater discrimination among the highly related Let-7 family members. Let-7f, Let-7g, and Let-71 were repressed 240, 90, and 195 fold, respectively, with other family members repressed to a lesser degree. Steady state levels of unrelated miRNAs were measured and exhibited no significant change. Importantly, the reduction in Let-7g was at a post-transcriptional step, as the level of the primary transcript was not decreased to the same magnitude (2.8 fold).

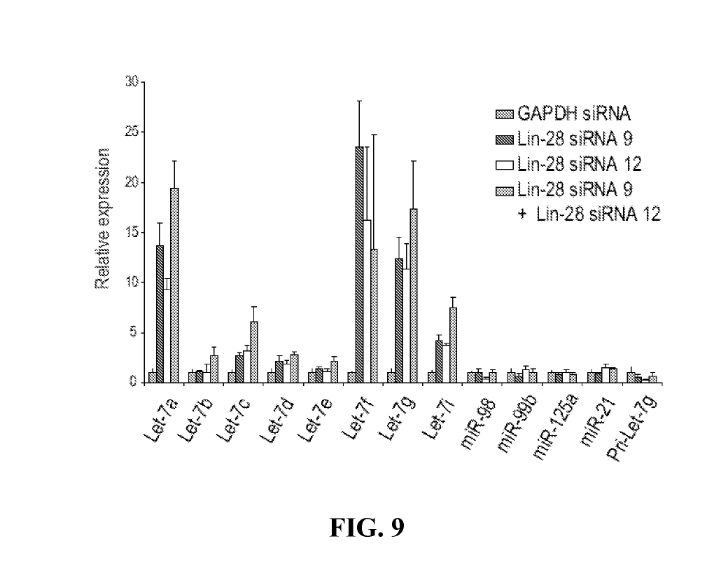

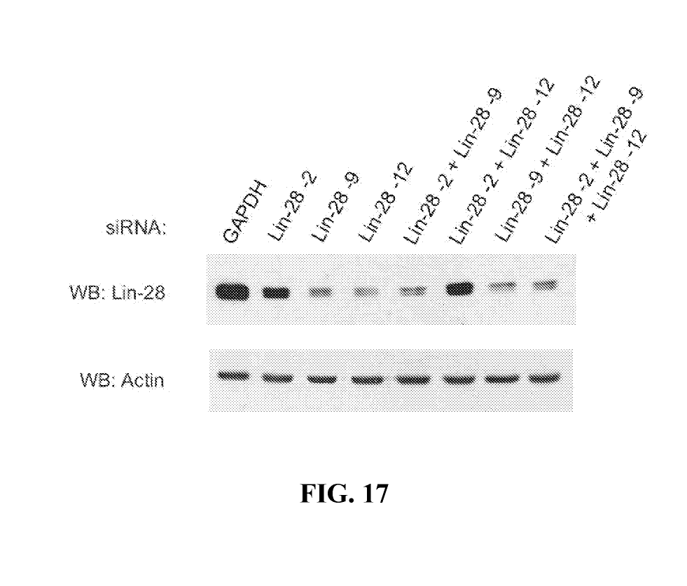

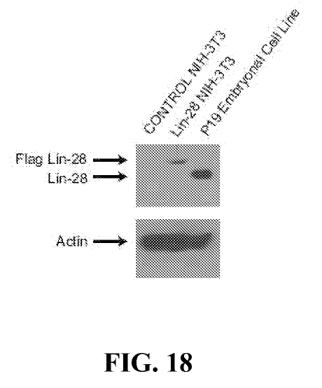

To further establish a role for Lin-28 in the repression of Let-7 expression, RNAi knockdown of Lin-28 was performed in P19 embryonic cells. Two siRNAs that reduced protein levels >90% released the processing block and led to increased mature Let-7 (FIG. 9 and FIG. 17).

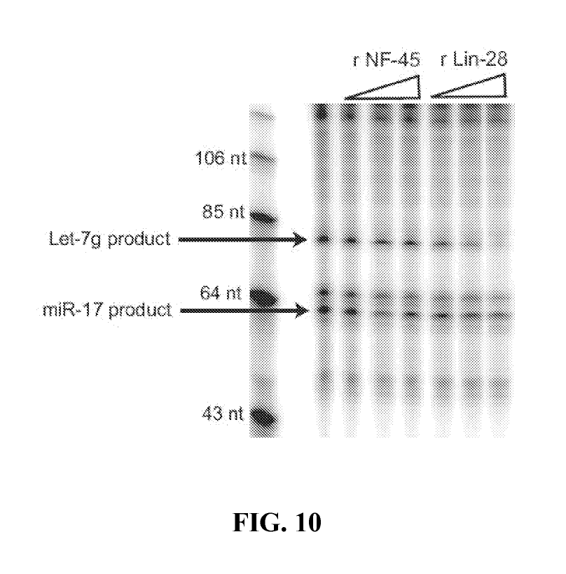

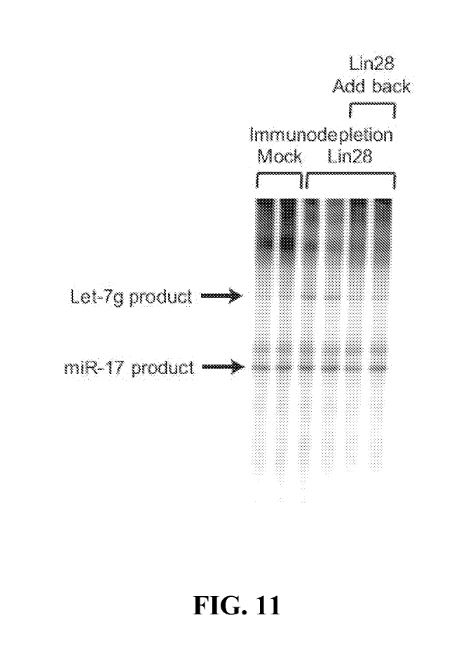

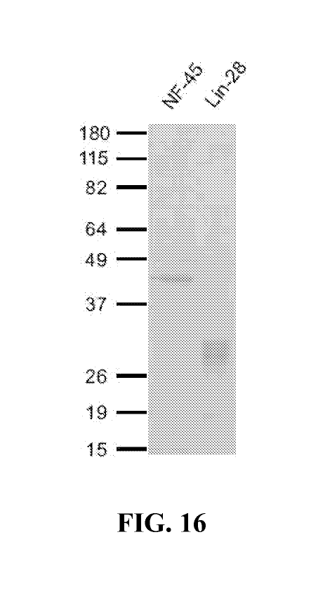

The final goal was to demonstrate the direct regulation of Let-7 processing by Lin-28. Recombinant Lin-28 and an unrelated RNA binding protein NF-45 were generated in E. coli and these purified proteins were introduced into our cell free Drosha assay (see FIG. 16 for recombinant proteins). As described above, Hela extracts will process Let-7g with similar efficiency as miR-17. As shown in FIG. 10, Lin-28 inhibited processing of Let-7g without affecting processing of miR-17. This was not a non-specific affect of RNA binding as the control protein (NF-45) had no effect on the processing of either substrate. To further support our conclusions, endogenous Lin-28 was removed from the P19 embryonic cell extract by immunodepletion. This enabled processing of Let-7 (FIG. 11).

These results outline a regulatory mechanism for the production of the Let-7 family of miRNAs. The primary transcripts for these miRNAs are uniformly expressed during embryonic development. The interaction of a Drosha inhibitor with the loop structure of the pri-miRNAs leads to a block at the Drosha processing step. Lin-28 is identified as a protein that blocks Let-7 processing. This RNA binding protein is highly expressed in early development and has been proposed as a marker for ES cells (Yang & Moss (2003) Gene Expr Patterns 3:719-726; Richards et al. (2004) Stem Cells 22:51-64). Differentiation of P19 cells with retinoic acid leads to robust induction of Let-7; Lin-28 protein decays at the time point that Let-7 processing is enabled (Lee et al. (2005) J Biol Chem 280:16635-16641; Wu & Belasco (2005) Mol Cell Biol 25:9198-9208). It is proposed that a similar decay of this protein during embryonic development is what allows Drosha processing of Let-7 and eventual production of the mature species.

In C. elegans the expression of lin-28 is regulated by the miRNAs lin-4 and let-7 (Moss, Lee & Ambros (1997) Cell 88:637-646; Morita & Han (2006) Embo J 25:5794-5804). Likewise, the mammalian Lin-28 mRNA is regulated by Let-7 and the lin-4 ortholog, miR-125 (Wu & Belasco (2005) Mol Cell Biol 25:9198-9208; Nelson et al. (2004) Rna 10:387-394; Moss & Tang (2003) Dev Biol 258:432-442). This suggests the presence of a positive feedback loop. The down-regulation of Lin-28 expression in differentiating P19 cells is largely due to transcriptional repression (Wu & Belasco (2005) Mol Cell Biol 25:9198-9208). After Lin-28 protein levels decay to a critical threshold, Let-7 primary transcripts escape the Drosha processing block and are converted to the mature form. Let-7 targeting leads to further repression of Lin-28, ensuring the continued production of Let-7. This is important for cell homeostasis, since aberrant expression of Lin-28 in differentiated cells would lead to a reduction of Let-7, an event that is frequently associated with carcinogenesis.

In cancer, as in normal development, the regulation of miRNA expression occurs in part at a post-transcriptional step (Thomson (2006) Genes Dev 20:2202-2207). Is this a result of Lin-28 re-expression? Published expression data has shown that Lin-28 is most abundant in stem cells (Richards et al. (2004) Stem Cells 22:51-64). In contrast, Lin-28B is expressed outside the stem compartment, and its expression has been observed in at least one tumor cell line (Guo et al. (2006) Gene 384:51-61).

The regulation and developmental expression of the mammalian Lin-28 closely parallels that of the C. elegans ortholog, suggesting it performs a similar function in that organism. Expression of let-7 during C. elegans larval development is known to be regulated at transcription (Johnson, Lin & Slack (2003) Dev Biol 259:364-379). In lin-28 mutants, however, the production of mature let-7 is accelerated by one larval stage. Similarly, in lin-4 mutants, which have elevated expression of lin-28, the production of mature let-7 is delayed (Johnson, Lin & Slack (2003) Dev Biol 259:364-379). Thus, it is possible that the mechanism we report here is conserved from C. elegans to mammals.

Let-7 has been connected to multiple cell growth and differentiation pathways (Esquela-Kerscher & Slack (2006) Nat Rev Cancer 6:259-269). The robust induction of this miRNA family during mammalian development suggests a role in promoting the differentiated state, and ectopic expression of Let-7 depletes progenitor cells out of tumor populations (Ibarra et al. (2007) Genes Dev 21:3238-3243; Yu et al. (2007) Cell 131:1109-1123). By this logic, Lin-28 would be essential to prevent precocious expression of Let-7 in stem cells, an event which would lead to differentiation and loss of pluripotency.

But does Lin-28 play an essential role in maintaining pluripotency? Loss of function studies in the mouse or in pluripotent cell lines have not been reported. However, a combination of Lin-28, Nanog, Oct-3/4, and Sox2 are sufficient to reprogram human somatic cells to pluripotent stem cells (Yu et al. (2007) Cell 131:1109-1123). The role of Nanog, Oct-3/4, and Sox2 in maintaining pluripotency is well established (Pan & Thomson (2007) Cell Res 17:42-49). The additional requirement for Lin-28 in these studies suggests that restoration of the Let-7 processing block is an essential step for reprogramming stem cells. These findings strongly implicate miRNA expression patterns as important determinants of stem cell fate.

### Materials and Methods

Drosha assays were performed essentially as described (Lee et al. (2002) Embo J 21:4663-4670). Assays were performed in nuclear extracts derived from P19 cells or Hela cells (Dignam (1990) Methods Enzymol 182:194-203). Pri-miRNA substrates were in vitro transcripts internally labeled with ³²P. In all assays, Let-7g and miR-17 primary transcripts were combined in the reaction to delivery internally controlled data. Products were isolated using Trizol reagent and were resolved on 8M urea/TBE/12.5% polyacrylamide gel electrophoresis. Identity of products was confirmed by size determination and by northern blotting. In some assays, RNA competitors were preincubated in the reaction. These RNAs were in vitro transcripts encoding the full loop region plus 12 nucleotides of duplexed stem. In some assays, recombinant purified protein was preincubated in the reaction. These proteins were expressed as 6× Histidine fusion proteins in E. coli or Flag-fusion proteins in HEK-293 cells, and affinity purified on Talon sepharose (Clontech) or M2-agarose (Sigma). In some assays, Lin-28 was immunodepleted from extracts before the reaction. Lin-28 polyclonal antibody (Abcam) was prebound to protein A sepharose. After extensive washing, resin was incubated with P19 extract for 18 hours at 4° C. The resultant extract was used for Drosha assays as described above. In some assays, immunopurified Drosha was used in place of extracts. Affinity purified Drosha polyclonal antibody (peptide sequence CPEEAEDIKK; SEQ ID NO: 15) was prebound to protein A sepharose. Resin was incubated in P19 nuclear extract 18 hours at 4° C. Beads were washed extensively in IP buffer. Let-7g and miR-17 pri-miRNA substrates were incubated with beads for 1 hour at 37° C. Drosha products were resolved as above.

Let-7 interacting proteins were isolated by RNA affinity capture. RNA oligonucleotide capture probes corresponding to the Let-7d loop or a random (control) sequence, fully 2′-O-methyl modified, 3′ Biotin linked, were bound to streptavidin agarose. Proteins were captured by incubation with P19 nuclear extract at 25° C. for 2 hours. Beads were washed extensively. Proteins were isolated and separated on a 4-20% polyacrylamide gel. Excised bands were identified by MALDI-TOF fingerprinting. Specific binding of Lin-28 to Let-7 was tested by crosslink analysis. Reactions were performed essentially as described (Myer, Fan & Steitz (1997) Embo J 16:2130-2139). In some cases, Lin-28 was isolated from crosslink reactions by denaturing immunoprecipitation (Hofmann et al. (1996) Genes Dev 10:2949-2959).

Lin-28 and Lin-28B expression was modulated using MSCV retroviral expression constructs or siRNA knockdown. siRNAs were obtained from Dharmacon and were transfected into P19 cells using Lipofectamine 2000. MSCV retrovirus was transduced into NIH-3T3 cells and cells were selected with puromycin. miRNA expression levels were characterized by microarray analysis and real time RT-PCR essentially as described (Thomson (2006) Genes Dev 20:2202-2207; Thomson et al. (2004) Nat Methods 1:47-53).

### Pri-microRNA Substrates

Substrate constructs (stem-loop with ˜10 nts of single-stranded RNA flanking region) were created by the polymerase chain reaction (PCR) using oligonucleotides as the DNA templates:

pri-Let-7g:
(SEQ ID NO: 16)
5′-TGC CTG ATT CCA GGC TGA GGT AGT CGT TTG TAC
AGT TTG AGG GTC TAT GAT ACC ACC CGG TAC AGG AGA
TAA CTG TAC AGG CAA CTG CCT TGC CAG GAA CAG CGC
G-3′
pri-miR-17:
(SEQ ID NO: 17)
5′-GTC AGA ATA ATG TCA AAG TGC TTA CAG TGC AGG
TAG TGA TAT GTG CAT CTA CTG CAG TGA AGG CAC TTG
TAG CAT TAT GGT GAC-3′

Forward and reverse primers added single-stranded RNA (ssRNA) flanks as well as 5′ Bgl-II and 3′ Xho-I restriction sites.

Primers for pri-Let-7g:
Forward
(SEQ ID NO: 18)
5′-GTC AAG ATC TCG TTT CCT TTT GCC TGA TTC CAG
GCT GA-3′
Reverse
(SEQ ID NO: 19)
5′-GTC ACT CGA GGG CAG CTG GCG CGC TGT TCC TGG
C-3′
Primers for pri-miR-17:
Forward-
(SEQ ID NO: 20)
5′-GTC AAG ATC TAT TGT GAC CAG TCA GAA TAA TGT
CAA AGT GCT TAC AG-3′
Reverse
(SEQ ID NO: 21)
5′-GTC ACT CGA GCG AGG CAG CTG TCA CCA TAA TGC
TAC AAG TGC CT-3′

Resulting PCR products were digested and cloned into a MSCV-splice-donor/splice-acceptor vector (SDSA3.0; J. M. Thomson and S. M. Hammond, unpublished results) based on the MSCV-puro vector (Clontech).

Pri-microRNA transcription templates were created from the pri-microRNA constructs described above; PCR was employed to add on a 5′ T7 promoter (5′-TCG TAA TAC GAC TCA CTA TAG GGT CCG CTA GCC TAG CTA CTA CCA-3′; SEQ ID NO: 22; and 5′-ATA AGT ATG ATA TTG TCA AGG AAA CCC-3′; SEQ ID NO: 23). Radiolabeled pri-microRNAs were created using T7 RNA polymerase (NEB) in accordance with the manufacturer's instructions. The resulting pri-microRNA substrates were (pri-microRNA sequences are in bold type; flanking vector sequences are italicized):

Pri-Let-7g
(SEQ ID NO: 24)
5′-TCC GCT AGC CTA GCT ACT ACC AGG TGA GTG GAG
ATC TCG TTT CCT TTT GCC TGA TTC CAG GCT GAG GTA
GTC GTT TGT ACA GTT TGA GGG TCT ATG ATA CCA CCC
GGT ACA GGA GAT AAC TGT ACA GGC AAC TGC CTT GCC
AGG AAC AGC GCG CCA GCT GCC CTC GAG GTT TAA ACG
AAT TCA GGG TTT CCT TGA CAA TAT CAT ACT TAT-3′
Pri-miR-17
(SEQ ID NO: 25)
5′-TCC GCT AGC CTA GCT ACT ACC AGG TGA GTG GAG
ATC TAT TGT GAC CAG TCA GAA TAA TGT CAA AGT GCT
TAC AGT GCA GGT AGT GAT ATG TGC ATC TAC TGC AGT
GAA GGC ACT TGT AGC ATT ATG GTG ACA GCT GCC TCG
CTC GAG GTT TAA ACG AAT TCA GGG TTT CCT TGA CAA
TAT CAT ACT TAT-3′

Cell Culture and Nuclear Extract Preparation

P19 cells were differentiated for 10 days in retinoic acid, as described (Thomson et al. (2006) Genes Dev 20:2202-2207). Undifferentiated and differentiated P19 cells, and Hela S3 nuclear extracts were prepared as previously described (Dignam (1990) Methods Enzymol 182:194-203). In brief, cell pellets, washed once in phosphate buffered saline (PBS), were resuspended in 2.5 volumes of Buffer A [10 mM HEPES, pH 7.9, 10 mM KCl, 1.5 mM MgCl₂, 0.2 mM EDTA, 1 mM dithiotreitol (DTT), 1 mM phenylmethylsulfonyl fluoride (PMSF), 20% glycerol and Complete protease inhibitors (CPI, Roche)] and incubated on ice for 10 minutes. Cells were then lysed using 5-10 passes in a dounce homogenizer. Nuclei were centrifuged at 1200 rpm for 5 minutes, followed by centrifugation 15,000×g for 15 minutes and the resulting supernatant was discarded. Pelleted nuclei were extracted in buffer C [20 mM HEPES, pH 7.9, 420 mM NaCl, 1.5 mM MgCl₂, 0.2 mM EDTA, 1 mM DTT, 1 mM PMSF, 20% glycerol and CPI] at 4° C. for 30 minutes (3 ml of buffer C was used per 1×10⁹ cells). Nuclear debris was then pelleted by centrifugation at 15,000 rpm for 30 minutes and the resulting nuclear extract was dialyzed for 5 hours against >50 volumes of buffer D [20 mM HEPES, pH 7.9, 100 mM KCl, 1.5 mM MgCl₂, 0.2 mM EDTA, 1 mM DTT, 1 mM PMSF and 20% glycerol).

### Cell Free Drosha Processing Assay

Drosha assay reactions were carried out as previously described with some modifications (Lee et al. (2002) Embo J 21:4663-4670, which is herein incorporated by reference in its entirety). 1×10⁵ cpm each of pri-Let-7g and pri-miR-17 transcripts were combined and incubated with 100 ng of nuclear extract (where indicated, either 100 ng P19 extract, 100 ng of differentiated P19 extract, 100 ng of Hela extract or 50 ng P19+50 μg HeLa extract)+0.1 n1 RNasin (Promega) for 30 minutes at 37° C. RNA was isolated using Trizol and precipitated in cold isopropanol. RNA pellets were washed once in 70% ethanol and resuspended in 15 μl of formamide loading buffer. Processed pri-microRNA products were resolved on a 12.5% acrylamide-8M urea sequencing gel and visualized by autoradiography using a Storm phosphoimager (GE Life Sciences); pre-microRNA band intensities were quantitated using Imagequant software (GE Life Sciences). A molecular weight ladder of radiolabeled, concatamerized RNA oligos was used to determine the size of the pre-microRNA products.

### Drosha Immunoprecipitation

For Drosha assays using immunoprecipitated Drosha, we first generated rabbit polyclonal anti-Drosha antibody using the peptide sequence CPEEAEDIKK (SEQ ID NO: 15). 250 μl of P19 nuclear extract was precleared with 25 μl protein-A sepharose and incubated either with 40 μl of affinity-purified Drosha antibody and 45 μl of packed protein-A sepharose or with protein-A sepharose alone. Igepal CA-630 (Sigma) was added to 0.5% v/v and immunoprecipitation reactions were incubated for 1 hour at 4° C. Beads were then washed three times in buffer D+0.5% Igepal. Individual Drosha reactions were carried out as described above using 10 μl of immobilized protein from each immunoprecipitation reaction, pri-miRNA substrates (individually or in combination), buffer D, and 1 μl RNasin.

### Drosha Assay Northern Blot

To identify pre-microRNA products, Drosha assays were carried out as described above except that 0.15 μg of unlabeled pri-microRNA was individually incubated in Hela extract. Separated RNA products were then transferred onto Nylon (Amersham) which was then UV cross-linked and hybridized to locked nucleic acid (LNA) probes (Integrated DNA Technologies) that were labeled on the 5′ end with T4 polynucleotide kinase (NEB). Hybridizations occurred overnight at 65° C., were washed three times at 65° C. in 0.4×SSC+0.2% SDS and cross-reacted bands were visualized by autoradiography.

Northern probe sequences (lower case denotes LNA; upper case denotes DNA)

| | |
|---|---|
| Let-7g: | |
| 5′-ACTgTaCaAaCgAcTaCcTcA-3′ | (SEQ ID NO: 26) |
| miR-17-3p: | |
| 5′-ACAaGtGcCtTcAcTgCaGt-3′ | (SEQ ID NO: 27) |

### Stem-Loop RNA Competitor Assay

Drosha reactions were pre-incubated on ice for 30 minutes with unlabeled microRNA stemloop RNAs added to final concentrations of 12.5 nM, 125 nM and 1250 nM, respectively. The competitor RNAs were either 2′-O-methlyated RNA oligos (Dharmacon), or were in vitro transcribed from long double stranded DNAs (dsDNAs) encoding a T7 promoter, the upper 10 basepairs of stem and the loop of the indicated pri-microRNA. The dsDNAs were created by annealing a T7 adapter primer to a longer microRNA primer, followed by a brief PCR extension step with Pfu DNA polymerase (Promega) (95° C., 5 minutes, 59° C., 1 minute, 72° C., 10 minutes for 1 cycle).

T7 adapter:
(SEQ ID NO: 28)
5′-gatgTAATACGACTCACTATAGGG-3′;
hsa-let-7d:
(SEQ ID NO: 29)
5′-caggtcgtatagttacctccttgtgggcaaaatccctgccctaaa
actatgcaaccccctatagtgagtcgtattaCATC-3;′
hsa-let-7a-2:
(SEQ ID NO: 30)
5′-gaggctgtacagttatacccttgatgtaattctaaactatacaac
cccctatagtgagtcgtattaCATC-3′;
hsa-let-7f-1:
(SEQ ID NO: 31)
5′-agattgtatagttatctcctgaacagggtaaaatcactaccccac
aactatacaatcccctatagtgagtcgtattaCATC-3′;
hsa-let-7c:
(SEQ ID NO: 32)
5′-aaggttgtacagttaactcccagggtgtaactctaaaccatacaa
ccccctatagtgagtcgtattaCATC-3′;
hsa-let-7a-3:
(SEQ ID NO: 33)
5′-tagattgtatagttatcccatagcagggcagagccccaaactata
caaccccctatagtgagtcgtattaCATC-3′;
hsa-let-7i:
(SEQ ID NO: 34)
5′-agcttgcgcagttatctccacagcgggcaatgtcacaacccgacc
aacagcacaaacccctatagtgagtcgtattaCATC-3′;
hsa-let-7f-2:
(SEQ ID NO: 35)
5′-tagactgtatagttataccaagatggggtatgaccctaaaactat
acaatcccctatagtgagtcgtattaCATC-3′;
hsa-let-7g:
(SEQ ID NO: 36)
5′-ggcctgtacagttatctcctgtaccgggtggtatcatagaccctc
aaactgtacaaacccctatagtgagtcgtattaCATC-3′;
hsa-let-7a-1:
(SEQ ID NO: 37)
5′-tagattgtatagttatctcccagtggtgggtgtgaccctaaaact
atacaaccccctatagtgagtcgtattaCATC-3′;
hsa-let-7e:
(SEQ ID NO: 38)
5′-gaggccgtatagtgatctccttgggtgtcctcctcaactatacaa
ccccctatagtgagtcgtattaCATC-3′;
hsa-let-7b:
(SEQ ID NO: 39)
5′-aggttgtatagttatcttccgaggggcaacatcactgccctgaaa
ccacacaaccccctatagtgagtcgtattaCATC-3′;
hsa-mir-17:
(SEQ ID NO: 40)
5′-tcactgcagtagatgcacatatcactacctgcactgtccctatag
tgagtcgtattaCATC-3′;
hsa-mir-20a:
(SEQ ID NO: 41)
5′-ctcataatgcagtagataactaaacactacctgcactatccctat
agtgagtcgtattaCATC-3′

### Lin-28 Immunodepletion Drosha Assay

Protein A-sepharose beads were washed 3 times in “Hi DTT” immunoprecipitation (IP) buffer [20 mM HEPES, pH 7.6, 2 mM MgCl₂, 150 mM NaCl, 10 mM DTT, 1 mM PMSF, 0.5% NP-40] followed by 5 washes in IP buffer [20 mM HEPES, pH 7.6, 2 mM MgCl₂, 150 mM NaCl, 1 mM DTT, 1 mM PMSF, 0.5% NP-40]. 15 μL of anti-Lin-28 antibody (Abeam) was prebound to 30 μL protein A sepharose beads (Sigma) for 1 hour at 25° C. in IP buffer, followed by extensive washing with buffer D+0.5% NP-40. 100 μL of P19 nuclear lysate (5 mg/ml) was diluted 2-fold in buffer D+2 μl of RNasin and rotated overnight at 4° C. with either the antibody-bead mixture or beads alone as a mock sample.

### Recombinant Protein Competitor Assay

Drosha assays were carried out as described above except that, where indicated, 2 ng, 20 ng, and 200 ng of recombinant protein was added to Hela extract/pri-microRNA mixtures before the 37° C. incubation step.

### RNA Affinity Pulldown

30 μl of 100 μM Let-7d loop biotinylated 2′-O-methylated RNA oligonucleotide, (5′-UUmAmGmGmGmCmAmGmGmGmAmUmUmUmUmGmCmCmCmAmCmAmAmG mGmAmGmGmU-18s-8i-3′; SEQ ID NO: 42; Dharmacon) or a non-specific control oligo (5′-Bi-18S-18S-mAmUmAmAmGmUmAmUmGmAmUmAmUmUmGmUmC-3′; SEQ ID NO: 43; Dharmacon) were bound to 150 μl of streptdavidin-agarose beads (Fluka) for 1 hour at 37° C. in “high salt” buffer D (+1M KCl). Bead-oligo mixtures were washed 3 times in buffer D+0.2% Triton X-100 (buffer D+T). 15 mg of P19 cell nuclear extract (equivalent to 1×10⁹ cells) was diluted up to 20 ml in buffer D+T, pre-cleared with streptavidin agarose beads and incubated with bead-oligo mixtures at 25° C. for 3 hours. Beads were then washed 6 times in Buffer D+T and bound proteins were eluted using SDS protein loading buffer+50 mM DTT. Proteins were separated on a 4-20% pre-cast polyacrylamide gel (Jule Biotechnologies) and stained with Coomassie brilliant blue G-250 (Bio-Rad); appropriate bands were excised and submitted to the UNC-Duke Michael Hooker Proteomics Facility for tryptic digestion and MALDI-TOF footprinting.

### UV Crosslinking

Cross-linking experiments were carried out essentially as previously described (Myer, Fan & Steitz (1997) Embo J 16:2130-2139). In brief, 50 μg of P19 nuclear lysate, 3 μg of yeast tRNA and 5×10⁵ cpm of hsa-Let-7d or hsa-miR-20a loop probes (same competitor RNA sequences as in the “stem-loop RNA competitor assay” above) were combined and adjusted to a volume of 14 μl with buffer D [20 mM HEPES, pH 7.9, 20% glycerol, 100 mM KCl, 1.5 mM MgCl₂, 0.2 mM EDTA, 1 mM DTT, 1 mM PMSF]. Reaction mixtures were incubated for 30 minutes at 25° C., followed by irradiation with 1 Joule of UV light. Each sample was then incubated with 10 μg RNase A for 30 minutes at 37° C. The samples were then either prepared for immunoprecipitation (see below) or boiled in SDS protein loading buffer for 5 minutes (“input” samples). Where indicated, 2′-O-methyl oligonucleotides were added to a final concentration of 12.5 nM, 125 nM or 1250 nM to the reaction mixture and pre-incubated at 25° C. for 30 minutes prior to the addition of the labeled probe.

2′-O-methyl competitor sequences:
Let-7d loop 2′-O-methyl:
(SEQ ID NO: 42)
5′-mUmUmAmGmGmGmCmAmGmGmGmAmUmUmUmUmGmCmCmCmAmCm
AmAmGmGmAmGmGmU-3′;
Non-specific 2′-O-methyl:
(SEQ ID NO: 44)
5′-mAmCmCmAmAmCmAmGmGmCmCmGmGmGmAmCmAmAmGmUmGmCm
AmmAUmAmAmC-3′

### Crosslinking Immunoprecipitations

RNase A-treated samples were boiled in TSD buffer [50 mM Tris-C1, pH 7.5, 1% SDS, 5 mM DTT] for 10 minutes and diluted 10-fold in TNN buffer [50 mM Tris-C1, pH 7.5, 250 mM NaCl, 5 mM EDTA, 0.5% NP-40 and complete protease inhibitor (Roche)]. Diluted lysates were pre-cleared with protein-A sepharose beads (Sigma) followed by immunoprecipitation overnight at 4° C. with anti-Lin-28 antibody (Abcam) and protein A-beads or with protein-A beads alone (“mock” immunoprecipitation). Protein-bound beads were washed extensively with cold TNN buffer; proteins were eluted by boiling SDS protein loading buffer and separated on a 12% SDS-PAGE gel which was subsequently fixed and dried. Radiolabeled proteins were visualized by autoradiography.

### Recombinant Proteins

cDNAs were amplified from full-length ESTs coding for NFAT-45 and Lin-28 as well as a partial EST for Lin-28B (Open Biosystems) using the following primers:

Lin-28:
(SEQ ID NO: 45)
5′-tac gaa ttc ACC ATG GAC TAC AAA GAC GAT GAC
GAC AAG GGC TCG GTG TCC AAC CA-3′;
(SEQ ID NO: 46)
5′-cat gcg gcc gcT CAA TTC TGG GCT TCT GGG-3′
Lin-28B:
(SEQ ID NO: 47)
5′-AGC CAG AAA AAC TGC CCG GGC TGG CAG AGG ACG
AAC CCC AGG TTC TGC ATG GC-3′;
(SEQ ID NO: 48)
5′-TGA CGA CAA GGC CGA AGG CGG GGC AAG CAA AGG
TGA AGA GCC AGA AAA ACT GCC CG-3′

The following primers were used to rebuild the 5′ end of the Lin-28B gene by overlap extension PCR:

(SEQ ID NO: 49)
5′-tac gaa ttc ACC ATG GAC TAC AAA GAC GAT GAC
GAC AAG GCC GAA G-3′
and
(SEQ ID NO: 50)
5′-cat gcg gcc gcC TAA GTC TTT TTC CGT TTC TGA
ATC A-3′
NFAT 45:
(SEQ ID NO: 51)
5′-tac gaa ttc ACC ATG GAC TAC AAA GAC GAT GAC
GAC AAG AGG GGT GAC AGA GGAC-3′;
(SEQ ID NO: 52)
5′-cat gcg gcc gcT CAC TCC TGA GTC TCC ATG C-3′

Flag-tagged cDNAs were cloned into pcDNA 3.0 using 5′ EcoRI and 3′ NotI restriction sites. cDNAs were subsequently subcloned into pMSCV-puro-IRES-GFP (He et al. (2005) Nature 435:828-833). For over-expression studies, NIH-3T3 cells were transduced using virus generated with the LinXE ecotropic packaging line. Cells were selected with puromycin. 10 days post-infection, total RNA and protein samples were isolated using Trizol and SDS loading buffer, respectively. microRNA expression levels were examined by quantitative real-time PCR (qRT-PCR) as well as by microRNA microarray as previously reported (Thomson et al. (2006) Genes Dev 20:2202-2207; Thomson et al. (2004) Nat Methods 1:47-53). Protein over-expression was verified by western blot with a Lin-28 antibody (Abeam).

### Purification of His-Tagged Lin-28 and NF-45

The following primers were used to amplify mouse coding sequences for NF-45 and Lin-28; 6×His-NF45 5′-GGC CAT CAT CAT CAT CAT CAC AGG GGT GAC AGA GGA-3′ (SEQ ID NO: 53); BsmFI_{—}6xHis_NF45_push 5′-CAG TGG GAC GCT GTC TCA CCA TGG GCC ATC ATC ATC-3′ (SEQ ID NO: 54); XhoI_NF45 5′-CAT GCT CGA GTC ACT CCT GAG TCT CCA TGC-3′ (SEQ ID NO: 55); 6x_His_Lin-28 5′-GGC CAT CAT CAT CAT CAT CAC GGC TCG GTG TCC AAC-3′ (SEQ ID NO: 56); NcoI_{—}6xHis_Lin-28_push 5′-ATA CCA TGG GCC ATC ATC ATC ATC AC-3′ (SEQ ID NO: 57); Lin-28_XhoI 5′-CAT CTC GAG TCA ATT CTG GGC TTC TGG G-3′ (SEQ ID NO: 58). PCR products were inserted into the pET28b vector (Novagen) after NcoI/XhoI (vector; Lin-28) or BsmFI/XhoI (NF45) digestion. BL21 (DE3) pRIL E. coli (Stratagene) served as the host for protein expression. Briefly, positive clones were grown in 1 liter of LB to a density of 0.6 OD₆₀₀ at which time, recombinant protein was induced by the addition of 0.25 mM IPTG and cells were allowed to grow for an additional 3 hours. Cells were harvested by centrifugation and cell pellets suspended in 30 ml Talon resin equilibration/wash buffer [50 mM sodium phosphate, pH 7.0; 300 mM NaCl (Clontech)]. Cells were lysed by sonication and cleared by centrifugation. The cell lysate was incubated with 5 ml of equilibrated Talon resin beads and allowed to bind at 4′C for 1 hour. The resin was batch washed 3×20 minutes in 30 ml of equilibration/wash buffer and applied to a disposable column. The protein was eluted along a 50 ml gradient of equilibration buffer containing 0-200 mM imidazole and fractions were collected. Pure fractions were combined and dialysed in buffer D [20 mM Hepes, pH 7.9; 100 mM KCl; 0.2 mM EDTA; 1.5 mM MgCl₂; 1 mM PMSF; 1 mM DTT; and 20% glycerol]. Aliquots were stored at −80′C.

### Purification of Flag-Tagged Recombinant Lin-28

Twenty 10 cm plates of HEK293 cells were transiently transfected with either Flag-tagged NFAT-45, Lin-28 or Lin-28B over-expression constructs using Fugene 6 (Roche) according to the manufacturer's instructions. After 48 hours, cells were harvested by scraping in cold PBS, centrifuged at 1200 rpm for 5 minutes and lysed in IP buffer. Lysates were pre-cleared with 50 μA of protein A beads for 1 hour followed by immunoprecipitation with 50 μA of M2 flag agarose beads (Sigma) overnight at 4° C. Beads were then pelleted and washed 10 times in cold buffer D followed by elution with Flag peptide (Sigma) at 400 μg/ml in buffer D. Purity of eluted proteins was verified by SDS-PAGE followed by Coomassie blue staining of the gel.

### Lin-28 Knockdown Studies

P19 cells were seeded in six-well plates at ˜40% confluency and 2 hours later, were transfected with siRNAs to either GAPDH or Lin-28 (Dharmacon) using Lipofectamine 2000 (Invitrogen) as instructed by the manufacturer. Total RNA and protein was isolated approximately 72 hours later using Trizol and SDS loading buffer, respectively. microRNA expression levels were examined by quantitative real-time PCR (qRT-PCR) (Thomson et al. (2006) Genes Dev 20:2202-2207). siRNA sequences:

| | |
|---|---|
| si#2: | |
| 5′-GGA GAC AGG UGC UAC AAC UUU-3′ | (SEQ ID NO: 59) |
| si#9: | |
| 5′-AAU GAC GUC UUG UGC GUU UUU-3′ | (SEQ ID NO: 60) |
| si#12: | |
| 5′-AAA UGU GUC UCA CGG GUU UUU-3′ | (SEQ ID NO: 61) |

Table 1 presents the sequences and corresponding SEQ ID NOs of the sequences present within the non-limiting embodiments presented herein.

TABLE 1
Polynucleotide sequences and corresponding SEQ ID NOs.

| | Corresponding | | | |
|---|---|---|---|---|
| SEQ ID | DNA SEQ | | | |
| NO. | ID NO. | Identifier | Description | Sequence |
| 1 | 78 | hsa-let- | Homo sapiens let-7d | 5′-UUA GGG CAG |
| | | 7d | microRNA loop | GGA UUU UGC CCA |
| | | | sequence | CAA GGA GGU-3′ |
| 2 | 79 | hsa-let- | Homo sapiens let- | 5′-UAG AAU UAC |
| | | 7a-2 | 7a-2 microRNA | AUC AAG GGA GAU-3′ |
| | | | loop sequence | |
| 3 | 80 | hsa-let- | Homo sapiens let-7f- | 5′-GUG GGG UAG |
| | | 7f-1 | 1 microRNA loop | UGA UUU UAC CCU |
| | | | sequence | GUU CAG GAG AU-3′ |
| 4 | 81 | hsa-let-7c | Homo sapiens let-7c | 5′-UAG AGU UAC |
| | | | microRNA loop | ACC CUG GGA GUU-3′ |
| | | | sequence | |
| 5 | 82 | hsa-let- | Homo sapiens let- | 5′-UGG GGC UCU |
| | | 7a-3 | 7a-3 microRNA | GCC CUG CUA UGG |
| | | | loop sequence | GAU-3′ |
| 6 | 83 | hsa-let- | microRNA loop | 5′-GGU CGG GUU |
| | | 7i; mmu- | sequence of Homo | GUG ACA UUG CCC |
| | | let-7i; | sapiens let-7i; Mus | GCU GUG GAG AU-3′ |
| | | gga-let-7i | musculus let-7i; and | |
| | | | Gallus gallus let-7i | |
| 7 | 84 | hsa-let- | Homo sapiens let-7f- | 5′-UUA GGG UCA |
| | | 7f-2 | 2 microRNA loop | UAC CCC AUC UUG |
| | | | sequence | GAG AU-3′ |
| 8 | 85 | hsa-let- | microRNA loop | 5′-UGA GGG UCU |
| | | 7g; mmu- | sequence of Homo | AUG AUA CCA CCC |
| | | let-7g; | sapiens let-7g, Mus | GGU ACA GGA GAU-3′ |
| | | gga-let- | musculus let-7g, | |
| | | 7g; bta- | Gallus gallus let-7g, | |
| | | let-7g | and Bos taurus let- | |
| | | | 7g | |
| 9 | 86 | hsa-let- | Homo sapiens let- | 5′-UUA GGG UCA |
| | | 7a-1 | 7a-1 microRNA | CAC CCA CCA CUG |
| | | | loop sequence | GGA GAU-3′ |
| 10 | 87 | hsa-let-7e | Homo sapiens let-7e | 5′-GAG GAG GAC |
| | | | microRNA loop | ACC CAA GGA GAU |
| | | | sequence | C-3′ |
| 11 | 88 | hsa-let- | Homo sapiens let-7b | 5′-UCA GGG CAG |
| | | 7b | microRNA loop | UGA UGU UGC CCC |
| | | | sequence | UCG GAA GAU-3′ |
| 12 | 89 | hsa-miR- | Homo sapiens miR- | 5′-GUG GGG UAG |
| | | 98 | 98 microRNA loop | GGA UAU UAG GCC |
| | | | sequence | CCA AUU AGA AGA |
| | | | | U-3′ |
| 13 | 90 | xtr-let-7g | Xenopus tropicalis | 5′-UAA GGG UCU |
| | | | let-7g microRNA | GUG ACA CCA CCC |
| | | | loop sequence | UCU GUU GGA GAU-3′ |
| 14 | 91 | Cel-let-7 | Caenorhabditis | 5′-UGG AAU AUU |
| | | | elegans let-7 | ACC ACC GGU G-3′ |
| | | | microRNA loop | |
| | | | sequence | |

### Example 2

### Lin-28 Blocks Dicer-Mediated Processing of Precursor miRNAs

Experiments were performed to determine if Lin-28 can block Dicer-mediated processing of precursor miRNAs, similar to the Lin-28-induced block on Drosha-mediated processing of primary miRNA transcripts.

RNA oligonucleotides comprising a polo-like kinase-1 (PLK-1) miRNA sequence and a Let-7a2 loop sequence or a PLK-1 miRNA sequence and a control loop sequence were end labeled with ³²P and incubated in HeLa cell cytoplasmic extracts with decreasing concentrations of recombinant Lin-28: 100 ng/μl, 30 ng/μl, 10 ng/μl, 1 ng/μl. Reactions were performed in Buffer D supplemented with 500 μM ATP. After a ninety minute incubation at 37° C., RNA was isolated and separated on a 15% acrylamide/UREA/TBE gel. Products were visualized by autoradiography.

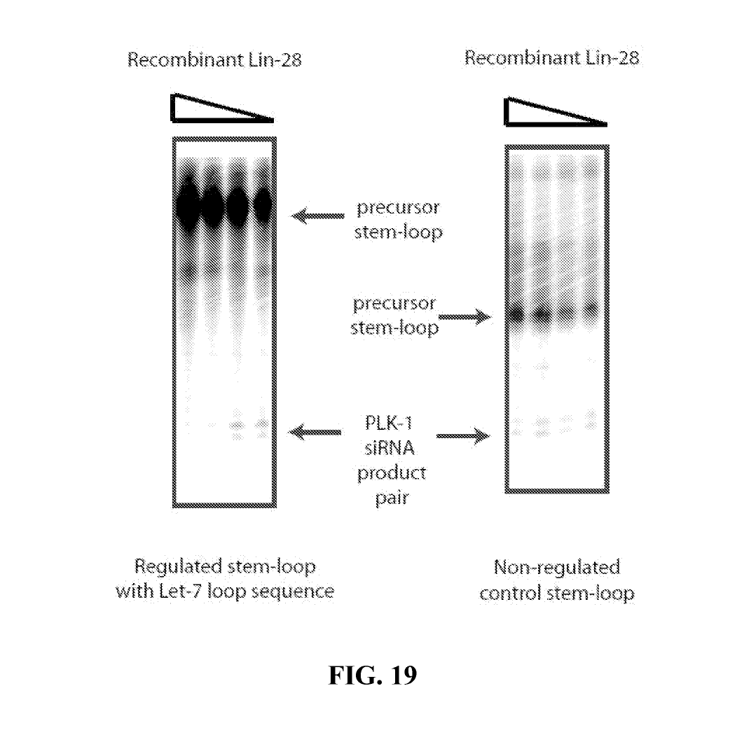

RNA oligonucleotides comprising a PLK-1 miRNA sequence and a Let-7a2 loop sequence are not efficiently converted to miRNA duplexes in the presence of recombinant Lin-28 (see left gel of FIG. 19). A control RNA oligonucleotide comprising the identical PLK-1 miRNA but with an artificial control loop sequence is converted to the miRNA duplex in the presence of identical concentrations of Lin-28 (see right gel of FIG. 19). The PLK-1 oligonucleotide with the Let-7a2 loop had the following sequence: agagacuuaggcacaaucuUUUAGAAUUACAUCAAGGGAGAUAAAGAUUGUGCCUAA GUCUCUuu, which is set forth in SEQ ID NO: 100. The PLK-1 oligonucleotide with the control loop sequence had the following sequence:

SEQ ID NO: 101
agagacuuaggcacaaucuUUCAAGAGAAGAUUGUGCCUAAGUCUCUuu,
which is set forth in.

1. An isolated polynucleotide comprising or encoding a nucleotide sequence having at least 70% sequence identity to any one of the sequences set forth in SEQ ID NO: 1, 2, 3, 4, 5, 6, 7, 8, 9, 10, 11, 12, 13, or 14, or an active fragment thereof, wherein said polynucleotide or an expression product thereof can not be processed to form a mature let-7 miRNA when introduced into or expressed in a cell, and wherein said polynucleotide promotes the processing of at least one of a regulated primary microRNA transcript by Drosha and a regulated precursor microRNA by Dicer when introduced into or expressed in a cell.

2. The isolated polynucleotide of claim 1, wherein said polynucleotide comprises or encodes the sequence set forth in SEQ ID NO: 1, 2, 3, 4, 5, 6, 7, 8, 9, 10, 11, 12, 13, or 14.

3. The isolated polynucleotide of claim 1, wherein said polynucleotide inhibits the activity of a Lin-28-like polypeptide when introduced into or expressed in a cell, wherein said activity comprises the inhibition of the processing of at least one of a regulated primary microRNA transcript by Drosha and a regulated precursor microRNA by Dicer.

4. The isolated polynucleotide of claim 3, wherein said polynucleotide inhibits the interaction between said Lin-28-like polypeptide and at least one of a regulated primary microRNA transcript and a regulated precursor microRNA when introduced into or expressed in a cell.

5. The isolated polynucleotide of claim 3, wherein said Lin-28-like polypeptide comprises Lin-28 or Lin-28B.

6. The isolated polynucleotide of claim 1, wherein said regulated primary microRNA transcript comprises a primary Let-7 microRNA transcript or said regulated precursor microRNA comprises a precursor Let-7 microRNA.

7. The isolated polynucleotide of claim 6, wherein said primary Let-7 microRNA transcript or precursor Let-7 microRNA comprises a microRNA selected from the group consisting of Let-7, Let-7a-1, Let-7a-2, Let-7a-3, Let-7b, Let-7c, Let-7d, Let-7e, Let-7f-1, Let-7f-2, Let-7g, Let-71, and miR-98.

8. An isolated polynucleotide wherein said polynucleotide or expression product thereof comprises a stem-loop structure and can be recognized and processed by Dicer or Drosha, wherein the loop of said stem-loop structure has a nucleotide sequence having at least 70% sequence identity to any one of the sequences set forth in SEQ ID NO: 1, 2, 3, 4, 5, 6, 7, 8, 9, 10, 11, 12, 13, or 14, or an active fragment thereof, wherein the stem of said stem-loop structure comprises a miRNA, wherein the loop has a sequence that is heterologous to the sequence of said stem of said stem-loop structure, and wherein the processing of said polynucleotide or expression product thereof by at least one of Dicer and Drosha is inhibited by a Lin-28-like polypeptide.

9. The isolated polynucleotide of claim 8, wherein said polynucleotide comprises or encodes the sequence set forth in SEQ ID NO: 1, 2, 3, 4, 5, 6, 7, 8, 9, 10, 11, 12, 13, or 14.

10. The isolated polynucleotide of claim 8, wherein a target polynucleotide of said miRNA is a disease-causing gene.

11. The isolated polynucleotide of claim 8, wherein said miRNA has at least 75% complementarity to the target mRNA of said miRNA.

12. The isolated polynucleotide of claim 8, wherein the sequence of said miRNA has at least 75% sequence identity to an endogenous microRNA.

13. The isolated polynucleotide of claim 8, wherein a target polynucleotide of said miRNA is selected from the group consisting of a polo-like kinase-1 (PLK1) gene, an origin recognition complex-2 (ORC2) gene, and a cell-divisional cycle-2 (CDC2) gene.

14-15. (canceled)

16. The isolated polynucleotide of claim 8, wherein a reduction in the expression level of a target polynucleotide of said miRNA suppresses the growth or viability of cells in which said isolated polynucleotide has been introduced or expressed.

17. The isolated polynucleotide of claim 8, wherein said loop of said stem-loop structure can interact with a Lin-28-like polypeptide.

18. The isolated polynucleotide of claim 8, wherein said Lin-28-like polypeptide comprises Lin-28 or Lin-28B.

19. A host cell comprising the polynucleotide of claim 8.

20. A pharmaceutical composition comprising the polynucleotide of claim 8 and a pharmaceutically acceptable carrier.

21. An expression cassette comprising a polynucleotide encoding a nucleotide sequence having at least 70% sequence identity to any one of the sequences set forth in SEQ ID NO: 1, 2, 3, 4, 5, 6, 7, 8, 9, 10, 11, 12, 13, or 14, or an active fragment thereof, wherein the expression product of said polynucleotide can not be processed to form a mature Let-7 miRNA, and wherein said polynucleotide promotes the processing of at least one of a regulated primary microRNA transcript by Drosha and a regulated precursor microRNA by Dicer when expressed in a cell.

22. The expression cassette of claim 21, wherein said polynucleotide encodes the sequence set forth in SEQ ID NO: 1, 2, 3, 4, 5, 6, 7, 8, 9, 10, 11, 12, 13, or 14.

23. The expression cassette of claim 21, wherein said polynucleotide inhibits the activity of a Lin-28-like polypeptide when expressed in a cell, wherein said activity comprises the inhibition of the processing of at least one of a regulated primary microRNA transcript by Drosha and a regulated precursor microRNA by Dicer.

24. The expression cassette of claim 23, wherein said polynucleotide inhibits the interaction between said Lin-28-like polypeptide and at least one of a regulated primary microRNA transcript and a regulated precursor miRNA when expressed in the cell.

25. The expression cassette of claim 23, wherein said Lin-28-like polypeptide comprises Lin-28 or Lin-28B.

26. The expression cassette of claim 21, wherein said regulated primary microRNA transcript comprises a primary Let-7 microRNA transcript or said regulated precursor microRNA comprises a precursor Let-7 microRNA.

27. The expression cassette of claim 26, wherein said primary Let-7 microRNA transcript or said precursor Let-7 microRNA comprises a microRNA that is selected from the group consisting of Let-7, Let-7a-1, Let-7a-2, Let-7a-3, Let-7b, Let-7c, Let-7d, Let-7e, Let-7f-1, Let-7f-2, Let-7g, Let-71, and miR-98.

28. An expression cassette comprising a polynucleotide, wherein the expression product of said polynucleotide comprises a stem-loop structure and can be recognized and processed by Drosha or Dicer, wherein the loop of said stem-loop structure has the nucleotide sequence having at least 70% sequence identity to any one of the sequences set forth in SEQ ID NO: 1, 2, 3, 4, 5, 6, 7, 8, 9, 10, 11, 12, 13, or 14, or an active fragment thereof, wherein the stem of said stem-loop structure comprises a miRNA, wherein the loop has a sequence that is heterologous to the sequence of said stem of said stem-loop structure, and wherein the processing of said expression product by at least one of Drosha and Dicer is inhibited by a Lin-28-like polypeptide.

29. The expression cassette of claim 28, wherein said polynucleotide encodes the sequence set forth in SEQ ID NO: 1, 2, 3, 4, 5, 6, 7, 8, 9, 10, 11, 12, 13, or 14.

30. The expression cassette of claim 28, wherein a target polynucleotide of said miRNA is a disease-causing gene.

31. The expression cassette of claim 28, wherein said miRNA has at least 75% complementarity to a target mRNA of said miRNA.

32. The expression cassette of claim 28, wherein the sequence of said miRNA has at least 75% sequence identity to an endogenous microRNA.

33. The expression cassette of claim 28, wherein a target polynucleotide of said miRNA is selected from the group consisting of a polo-like kinase-1 (PLK1) gene, an origin recognition complex-2 (ORC2) gene, and a cell-divisional cycle-2 (CDC2) gene.

34-35. (canceled)

36. The expression cassette of claim 28, wherein a reduction in the expression level of a target polynucleotide of said miRNA suppresses the growth or viability of cells in which said expression cassette has been expressed.

37. The expression cassette of claim 28, wherein said loop of said stem-loop structure can interact with a Lin-28-like polypeptide.

38. The expression cassette of claim 28, wherein said Lin-28-like polypeptide comprises Lin-28 or Lin-28B.

39. (canceled)

40. A host cell comprising the expression cassette of claim 28.

41. A pharmaceutical composition comprising the expression cassette of claim 28 and a pharmaceutically acceptable carrier.

42. A method for inhibiting the activity of a Lin-28-like polypeptide, said method comprising contacting a cell with a compound, wherein said compound inhibits the Lin-28-like polypeptide-mediated inhibition of the processing of at least one of a regulated primary microRNA transcript by Drosha and a regulated precursor microRNA by Dicer, thereby inhibiting the activity of said Lin-28-like polypeptide.

43. A method for treating a cancer in a subject, said method comprising administering to said subject an effective amount of a compound, wherein said compound inhibits the activity of a Lin-28-like polypeptide, wherein said activity comprises the inhibition of the processing of at least one of a regulated primary microRNA transcript by Drosha and a regulated precursor microRNA by Dicer.

44. The method of claim 42, wherein said compound binds to said Lin-28-like polypeptide.

45. The method of claim 42, wherein said compound inhibits the interaction between said Lin-28-like polypeptide and at least one of a regulated primary microRNA transcript and a regulated precursor microRNA.

46. The method of claim 42, wherein said Lin-28-like polypeptide comprises Lin-28 or Lin-28B.

47. The method of claim 42, wherein said regulated primary microRNA transcript comprises a primary Let-7 microRNA transcript or said regulated precursor microRNA comprises a precursor Let-7 microRNA.

48. The method of claim 47, wherein said primary Let-7 microRNA transcript or said precursor Let-7 microRNA comprise a microRNA selected from the group consisting of Let-7, Let-7a-1, Let-7a-2, Let-7a-3, Let-7b, Let-7c, Let-7d, Let-7e, Let-7f-1, Let-7f-2, Let-7g, Let-71, and miR-98.

49. The method of claim 42, wherein said compound comprises a polynucleotide that when introduced into or expressed in a cell inhibits said activity of said Lin-28-like polypeptide.

50. The method of claim 49, wherein said polynucleotide comprises or encodes a sequence of a loop of a Lin-28-like polypeptide-regulated primary microRNA transcript or a Lin-28-like polypeptide-regulated precursor microRNA or an active variant or fragment thereof.

51. The method of claim 49, wherein said polynucleotide comprises or encodes a nucleotide sequence having at least 70% sequence identity to any one of the sequences set forth in SEQ ID NO:1, 2, 3, 4, 5, 6, 7, 8, 9, 10, 11, 12, 13, or 14, or an active fragment thereof, and wherein said polynucleotide or an expression product thereof can not be processed to form a mature Let-7 miRNA when introduced into or expressed in a cell.

52. The method of claim 51, wherein said polynucleotide comprises or encodes the sequence set forth in SEQ ID NO:1, 2, 3, 4, 5, 6, 7, 8, 9, 10, 11, 12, 13, or 14.

53. A method for treating a disease in a subject, said method comprising administering to said subject an effective amount of a polynucleotide, wherein said polynucleotide or expression product thereof comprises a stem-loop structure and can be recognized and processed by at least one of Dicer and Drosha, wherein the loop of said stem-loop structure has a nucleotide sequence of a loop of a Lin-28-like polypeptide-regulated primary microRNA transcript, a Lin-28-like polypeptide regulated precursor miRNA, or an active variant or fragment thereof, wherein the stem of said stem-loop structure comprises a miRNA, wherein a target polynucleotide of said miRNA is a disease-causing gene, wherein the loop has a sequence that is heterologous to the sequence of said stem of said stem-loop structure, and wherein the processing of said polynucleotide or expression product thereof by at least one of Dicer and Drosha is inhibited by a Lin-28-like polypeptide.

54. The method of claim 53, wherein said loop has a nucleotide sequence having at least 70% sequence identity to any one of the sequences set forth in SEQ ID NO:1, 2, 3, 4, 5, 6, 7, 8, 9, 10, 11, 12, 13, or 14, or an active fragment thereof.

55. The method of claim 54, wherein said loop has the nucleotide sequence set forth in SEQ ID NO: 1, 2, 3, 4, 5, 6, 7, 8, 9, 10, 11, 12, 13, or 14.

56. The method of claim 53, wherein said disease comprises a cancer, and wherein said disease-causing gene comprises an oncogene.

57. The method of claim 53, wherein said loop of said stem-loop structure can interact with a Lin-28-like polypeptide.

58. The method of claim 53, wherein said Lin-28-like polypeptide comprises Lin-28 or Lin-28B.

59. A method for differentiating a cell or inhibiting the growth of a cell, said method comprising introducing into a cell a polynucleotide comprising or encoding a nucleotide sequence having at least 70% sequence identity to any one of the sequences set forth in SEQ ID NO:1, 2, 3, 4, 5, 6, 7, 8, 9, 10, 11, 12, 13, or 14, or an active fragment thereof, wherein said polynucleotide or an expression product thereof can not be processed to form a mature Let-7 miRNA when introduced into or expressed in a cell, and wherein said polynucleotide promotes the processing of at least one of a regulated primary microRNA transcript by Drosha and a regulated precursor microRNA by Dicer when introduced into or expressed in a cell.

60. The method of claim 59, wherein said polynucleotide comprises or encodes the sequence set forth in SEQ ID NO:1, 2, 3, 4, 5, 6, 7, 8, 9, 10, 11, 12, 13, or 14.

61. The method of claim 59, wherein said polynucleotide inhibits the activity of a Lin-28-like polypeptide when introduced into or expressed in a cell, wherein said activity comprises the inhibition of the processing of at least one of a regulated primary microRNA transcript by Drosha and a regulated precursor microRNA by Dicer.

62. The method of claim 59, wherein said polynucleotide inhibits the interaction between said Lin-28-like polypeptide and at least one of a regulated primary microRNA transcript and a regulated precursor microRNA when introduced into or expressed in a cell.

63. The method of claim 59, wherein said Lin-28-like polypeptide comprises Lin-28 or Lin-28B.

64. The method of claim 59, wherein said regulated primary microRNA transcript comprises a primary Let-7 microRNA transcript or said regulated precursor microRNA comprises a precursor Let-7 microRNA.

65. The method of claim 64, wherein said primary Let-7 microRNA transcript or said precursor Let-7 microRNA comprises a microRNA selected from the group consisting of Let-7, Let-7a-1, Let-7a-2, Let-7a-3, Let-7b, Let-7c, Let-7d, Let-7e, Let-7f-1, Let-7f-2, Let-7g, Let-71, and miR-98.

66. A method for selectively suppressing the expression of a target polynucleotide in a population of cells, said method comprising introducing a polynucleotide into said cells, wherein said polynucleotide or an expression product thereof comprises a stem-loop structure and can be recognized and processed by Drosha or Dicer, wherein the loop of said stem-loop structure has a nucleotide sequence of a loop of a Lin-28-like polypeptide-regulated primary microRNA transcript, a Lin-28-like polypeptide-regulated precursor miRNA, or an active variant or fragment thereof, wherein the stem of said stem-loop structure comprises a miRNA, wherein the loop sequence is heterologous to said stem sequence, and wherein the level of suppression of a target polynucleotide by said miRNA upon introduction or expression of said polynucleotide in a cell is negatively correlated with the expression level of a Lin-28-like polypeptide in said cell.

67. The method of claim 66, wherein said loop has a nucleotide sequence having at least 70% sequence identity to any one of the sequences set forth in SEQ ID NO: 1, 2, 3, 4, 5, 6, 7, 8, 9, 10, 11, 12, 13, or 14, or an active fragment thereof.

68. The method of claim 67, wherein said loop sequence has the nucleotide sequence set forth in SEQ ID NO: 1, 2, 3, 4, 5, 6, 7, 8, 9, 10, 11, 12, 13, or 14.

69. The method of claim 66, wherein said polynucleotide comprises a single-stranded RNA oligonucleotide.

70. The method of claim 66, wherein a target polynucleotide of said miRNA is a disease-causing gene.

71. The method of claim 66, wherein said miRNA has at least 75% complementarity to a target mRNA of said miRNA.

72. The method of claim 66, wherein the sequence of said miRNA has at least 75% sequence identity to an endogenous microRNA.

73. The method of claim 66, wherein said loop of said stem-loop structure can interact with a Lin-28-like polypeptide.

74. The method of claim 66, wherein said Lin-28-like polypeptide is Lin-28 or Lin-28B.

75. The method of claim 66, wherein said population of cells comprises stem cells, wherein a reduction in the expression level of a target polynucleotide of said microRNA suppresses the growth or viability of cells in which said polynucleotide has been introduced or expressed, thereby enriching the population of cells for stem cells.

76. The method of claim 75, wherein said stem cells comprise induced pluripotent stem cells.

77. The method of claim 75, wherein a target polynucleotide of said miRNA is selected from the group consisting of a polo-like kinase-1 (PLK1) gene, an origin recognition complex-2 (ORC2) gene, and a cell-divisional cycle-2 (CDC2) gene.

78. A method for enriching a population of cells for stem cells, wherein said method comprises introducing a polynucleotide into cells of said population of cells, wherein said population of cells comprises stem cells, wherein said polynucleotide or an expression product thereof comprises a stem-loop structure and can be recognized and processed by Drosha or Dicer, wherein the loop of said stem-loop structure has a nucleotide sequence of a loop of a Lin-28-like polypeptide-regulated primary microRNA transcript, a Lin-28-like polypeptide-regulated precursor miRNA, or an active variant or fragment thereof, wherein the stem of said stem-loop structure comprises a miRNA, wherein the loop sequence is heterologous to said stem sequence, wherein the level of suppression of a target polynucleotide by said miRNA upon introduction or expression of said polynucleotide in a cell is negatively correlated with the expression level of a Lin-28-like polypeptide in said cell, and wherein a reduction in the expression level of a target polynucleotide of said microRNA suppresses the growth or viability of cells in which said polynucleotide has been introduced or expressed, thereby enriching the population of cells for stem cells.

79. The method of claim 78, wherein said stem cells comprise induced pluripotent stem cells.

80. A host cell comprising the polynucleotide of claim 1.

81. A pharmaceutical composition comprising the polynucleotide of claim 1 and a pharmaceutically acceptable carrier.

82. A host cell comprising the expression cassette of claim 21.

83. A pharmaceutical composition comprising the expression cassette of claim 21 and a pharmaceutically acceptable carrier.


SEQUENCE LISTING (102 sequences)

SEQ ID NO: 1 - RNA, 30 nt, Homo sapiens
uuagggcagggauuuugcccacaaggaggu

SEQ ID NO: 2 - RNA, 21 nt, Homo sapiens
uagaauuacaucaagggagau

SEQ ID NO: 3 - RNA, 32 nt, Homo sapiens
gugggguagugauuuuacccuguucaggagau

SEQ ID NO: 4 - RNA, 21 nt, Homo sapiens
uagaguuacacccugggaguu

SEQ ID NO: 5 - RNA, 24 nt, Homo sapiens
uggggcucugcccugcuaugggau

SEQ ID NO: 6 - RNA, 32 nt, Homo sapiens
ggucggguugugacauugcccgcuguggagau

SEQ ID NO: 7 - RNA, 26 nt, Homo sapiens
uuagggucauaccccaucuuggagau

SEQ ID NO: 8 - RNA, 33 nt, Homo sapiens
ugagggucuaugauaccacccgguacaggagau

SEQ ID NO: 9 - RNA, 27 nt, Homo sapiens
uuagggucacacccaccacugggagau

SEQ ID NO: 10 - RNA, 22 nt, Homo sapiens
gaggaggacacccaaggagauc

SEQ ID NO: 11 - RNA, 30 nt, Homo sapiens
ucagggcagugauguugccccucggaagau

SEQ ID NO: 12 - RNA, 34 nt, Homo sapiens
gugggguagggauauuaggccccaauuagaagau

SEQ ID NO: 13 - RNA, 33 nt, Xenopus tropicalis
uaagggucugugacaccacccucuguuggagau

SEQ ID NO: 14 - RNA, 19 nt, Caenorhabditis elegans
uggaauauuaccaccggug

SEQ ID NO: 15 - protein, 10 aa, Homo sapiens
CPEEAEDIKK

SEQ ID NO: 16 - DNA, 106 bp, Artificial Sequence
  Oligonucleotide template
tgcctgattccaggctgaggtagtcgtttgtacagtttgagggtctatgataccacccggtacaggagataactgtacaggcaactgccttgccaggaacagcgcg

SEQ ID NO: 17 - DNA, 84 bp, Artificial Sequence
  Oligonucleotide template
gtcagaataatgtcaaagtgcttacagtgcaggtagtgatatgtgcatctactgcagtgaaggcacttgtagcattatggtgac

SEQ ID NO: 18 - DNA, 38 bp, Artificial Sequence
  Oligonucleotide primer
gtcaagatctcgtttccttttgcctgattccaggctga

SEQ ID NO: 19 - DNA, 34 bp, Artificial Sequence
  Oligonucleotide primer
gtcactcgagggcagctggcgcgctgttcctggc

SEQ ID NO: 20 - DNA, 47 bp, Artificial Sequence
  Oligonucleotide primer
gtcaagatctattgtgaccagtcagaataatgtcaaagtgcttacag

SEQ ID NO: 21 - DNA, 44 bp, Artificial Sequence
  Oligonucleotide primer
gtcactcgagcgaggcagctgtcaccataatgctacaagtgcct

SEQ ID NO: 22 - DNA, 45 bp, Artificial Sequence
  Oligonucleotide primer
tcgtaatacgactcactatagggtccgctagcctagctactacca

SEQ ID NO: 23 - DNA, 27 bp, Artificial Sequence
  Oligonucleotide primer
ataagtatgatattgtcaaggaaaccc

SEQ ID NO: 24 - DNA, 210 bp, Artificial Sequence
  Oligonucleotide substrate
tccgctagcctagctactaccaggtgagtggagatctcgtttccttttgcctgattccaggctgaggtagtcgtttgtacagtttgagggtctatgataccacccggtacaggagataactgtacaggcaactgccttgccaggaacagcgcgccagctgccctcgaggtttaaacgaattcagggtttccttgacaatatcatacttat

SEQ ID NO: 25 - DNA, 189 bp, Artificial Sequence
  Oligonucleotide substrate
tccgctagcctagctactaccaggtgagtggagatctattgtgaccagtcagaataatgtcaaagtgcttacagtgcaggtagtgatatgtgcatctactgcagtgaaggcacttgtagcattatggtgacagctgcctcgctcgaggtttaaacgaattcagggtttccttgacaatatcatacttat

SEQ ID NO: 26 - DNA, 21 bp, Artificial Sequence
  Oligonucleotide probe
actgtacaaacgactacctca

SEQ ID NO: 27 - DNA, 20 bp, Artificial Sequence
  Oligonucleotide probe
acaagtgccttcactgcagt

SEQ ID NO: 28 - DNA, 24 bp, Artificial Sequence
  Oligonucleotide primer
gatgtaatacgactcactataggg

SEQ ID NO: 29 - DNA, 80 bp, Artificial Sequence
  Oligonucleotide primer
caggtcgtatagttacctccttgtgggcaaaatccctgccctaaaactatgcaaccccctatagtgagtcgtattacatc

SEQ ID NO: 30 - DNA, 71 bp, Artificial Sequence
  Oligonucleotide primer
gaggctgtacagttatctcccttgatgtaattctaaactatacaaccccctatagtgagtcgtattacatc

SEQ ID NO: 31 - DNA, 81 bp, Artificial Sequence
  Oligonucleotide primer
agattgtatagttatctcctgaacagggtaaaatcactaccccacaactatacaatcccctatagtgagtcgtattacatc

SEQ ID NO: 32 - DNA, 71 bp, Artificial Sequence
  Oligonucleotide primer
aaggttgtacagttaactcccagggtgtaactctaaaccatacaaccccctatagtgagtcgtattacatc

SEQ ID NO: 33 - DNA, 74 bp, Artificial Sequence
  Oligonucleotide primer
tagattgtatagttatcccatagcagggcagagccccaaactatacaaccccctatagtgagtcgtattacatc

SEQ ID NO: 34 - DNA, 81 bp, Artificial Sequence
  Oligonucleotide primer
agcttgcgcagttatctccacagcgggcaatgtcacaacccgaccaacagcacaaacccctatagtgagtcgtattacatc

SEQ ID NO: 35 - DNA, 76 bp, Artificial Sequence
  Oligonucleotide primer
tagactgtatagttatctccaagatggggtatgaccctaaaactatacaatcccctatagtgagtcgtattacatc

SEQ ID NO: 36 - DNA, 82 bp, Artificial Sequence
  Oligonucleotide primer
ggcctgtacagttatctcctgtaccgggtggtatcatagaccctcaaactgtacaaacccctatagtgagtcgtattacatc

SEQ ID NO: 37 - DNA, 77 bp, Artificial Sequence
  Oligonucleotide primer
tagattgtatagttatctcccagtggtgggtgtgaccctaaaactatacaaccccctatagtgagtcgtattacatc

SEQ ID NO: 38 - DNA, 71 bp, Artificial Sequence
  Oligonucleotide primer
gaggccgtatagtgatctccttgggtgtcctcctcaactatacaaccccctatagtgagtcgtattacatc

SEQ ID NO: 39 - DNA, 79 bp, Artificial Sequence
  Oligonucleotide primer
aggttgtatagttatcttccgaggggcaacatcactgccctgaaaccacacaaccccctatagtgagtcgtattacatc

SEQ ID NO: 40 - DNA, 61 bp, Artificial Sequence
  Oligonucleotide primer
tcactgcagtagatgcacatatcactacctgcactgtccctatagtgagtcgtattacatc

SEQ ID NO: 41 - DNA, 63 bp, Artificial Sequence
  Oligonucleotide primer
ctcataatgcagtagataactaaacactacctgcactatccctatagtgagtcgtattacatc

SEQ ID NO: 42 - RNA, 30 nt, Artificial Sequence
  Oligonucleotide
uungggcngggnuuuugcccncnnggnggu

SEQ ID NO: 43 - RNA, 17 nt, Artificial Sequence
  Oligonucleotide
nunngunugnunuuguc

SEQ ID NO: 44 - RNA, 28 nt, Artificial Sequence
  Oligonucleotide
nccnncnggccgggncnngugcnnunnc

SEQ ID NO: 45 - DNA, 56 bp, Artificial Sequence
  Oligonucleotide primer
tacgaattcaccatggactacaaagacgatgacgacaagggctcggtgtccaacca

SEQ ID NO: 46 - DNA, 30 bp, Artificial Sequence
  Oligonucleotide primer
catgcggccgctcaattctgggcttctggg

SEQ ID NO: 47 - DNA, 53 bp, Artificial Sequence
  Oligonucleotide primer
agccagaaaaactgcccgggctggcagaggacgaaccccaggttctgcatggc

SEQ ID NO: 48 - DNA, 56 bp, Artificial Sequence
  Oligonucleotide primer
tgacgacaaggccgaaggcggggcaagcaaaggtgaagagccagaaaaactgcccg

SEQ ID NO: 49 - DNA, 46 bp, Artificial Sequence
  Oligonucleotide primer
tacgaattcaccatggactacaaagacgatgacgacaaggccgaag

SEQ ID NO: 50 - DNA, 37 bp, Artificial Sequence
  Oligonucleotide primer
catgcggccgcctaagtctttttccgtttctgaatca

SEQ ID NO: 51 - DNA, 55 bp, Artificial Sequence
  Oligonucleotide primer
tacgaattcaccatggactacaaagacgatgacgacaagaggggtgacagaggac

SEQ ID NO: 52 - DNA, 31 bp, Artificial Sequence
  Oligonucleotide primer
catgcggccgctcactcctgagtctccatgc

SEQ ID NO: 53 - DNA, 36 bp, Artificial Sequence
  Oligonucleotide primer
ggccatcatcatcatcatcacaggggtgacagagga

SEQ ID NO: 54 - DNA, 36 bp, Artificial Sequence
  Oligonucleotide primer
cagtgggacgctgtctcaccatgggccatcatcatc

SEQ ID NO: 55 - DNA, 30 bp, Artificial Sequence
  Oligonucleotide primer
catgctcgagtcactcctgagtctccatgc

SEQ ID NO: 56 - DNA, 36 bp, Artificial Sequence
  Oligonucleotide primer
ggccatcatcatcatcatcacggctcggtgtccaac

SEQ ID NO: 57 - DNA, 26 bp, Artificial Sequence
  Oligonucleotide primer
ataccatgggccatcatcatcatcac

SEQ ID NO: 58 - DNA, 28 bp, Artificial Sequence
  Oligonucleotide primer
catctcgagtcaattctgggcttctggg

SEQ ID NO: 59 - RNA, 21 nt, Artificial Sequence
  siRNA
ggagacaggugcuacaacuuu

SEQ ID NO: 60 - RNA, 21 nt, Artificial Sequence
  siRNA
aaugacgucuugugcguuuuu

SEQ ID NO: 61 - RNA, 21 nt, Artificial Sequence
  siRNA
aaaugugucucacggguuuuu

SEQ ID NO: 62 - RNA, 69 nt, Homo sapiens
ugagguaguagguuguauaguuuggggcucugcccugcuaugggauaacuauacaaucuacugucuuuc

SEQ ID NO: 63 - RNA, 77 nt, Homo sapiens
ugagguaguagauuguauaguugugggguagugauuuuacccuguucaggagauaacuauacaaucuauugccuucc

SEQ ID NO: 64 - RNA, 75 nt, Homo sapiens
ugagguaguagguugugugguuucagggcagugauguugccccucggaagauaacuauacaaccuacugccuucc

SEQ ID NO: 65 - RNA, 71 nt, Homo sapiens
ugagguaguagauuguauaguuuuagggucauaccccaucuuggagauaacuauacagucuacugucuuuc

SEQ ID NO: 66 - RNA, 72 nt, Homo sapiens
ugagguaguagguuguauaguuuuagggucacacccaccacugggagauaacuauacaaucuacugucuuuc

SEQ ID NO: 67 - RNA, 66 nt, Homo sapiens
ugagguaguagguuguauaguuuagaauuacaucaagggagauaacuguacagccuccuagcuuuc

SEQ ID NO: 68 - RNA, 66 nt, Homo sapiens
ugagguaguagguuguaugguuuagaguuacacccugggaguuaacuguacaaccuucuagcuuuc

SEQ ID NO: 69 - RNA, 66 nt, Homo sapiens
ugagguaggagguuguauaguugaggaggacacccaaggagaucacuauacggccuccuagcuuuc

SEQ ID NO: 70 - RNA, 75 nt, Homo sapiens
agagguaguagguugcauaguuuuagggcagggauuuugcccacaaggagguaacuauacgaccugcugccuuuc

SEQ ID NO: 71 - RNA, 77 nt, Homo sapiens
ugagguaguaguuugugcuguuggucggguugugacauugcccgcuguggagauaacugcgcaagcuacugccuugc

SEQ ID NO: 72 - RNA, 78 nt, Homo sapiens
ugagguaguaguuuguacaguuugagggucuaugauaccacccgguacaggagauaacuguacaggccacugccuugc

SEQ ID NO: 73 - RNA, 78 nt, Mus musculus; identical to SEQ ID NO: 72

SEQ ID NO: 74 - RNA, 78 nt, Gallus gallus; identical to SEQ ID NO: 72

SEQ ID NO: 75 - RNA, 78 nt, Bos taurus; identical to SEQ ID NO: 72

SEQ ID NO: 76 - RNA, 78 nt, Xenopus tropicalis
ugagguaguuguuuguacaguuuaagggucugugacaccacccucuguuggagauaacuguacaggccacugccuugc

SEQ ID NO: 77 - RNA, 64 nt, Caenorhabditis elegans
ugagguaguagguuguauaguuuggaauauuaccaccggugaacuaugcaauuuucuaccuuac

SEQ ID NO: 78 - DNA, 30 bp, Homo sapiens
ttagggcagggattttgcccacaaggaggt

SEQ ID NO: 79 - DNA, 21 bp, Homo sapiens
tagaattacatcaagggagat

SEQ ID NO: 80 - DNA, 32 bp, Homo sapiens
gtggggtagtgattttaccctgttcaggagat

SEQ ID NO: 81 - DNA, 21 bp, Homo sapiens
tagagttacaccctgggagtt

SEQ ID NO: 82 - DNA, 24 bp, Homo sapiens
tggggctctgccctgctatgggat

SEQ ID NO: 83 - DNA, 32 bp, Homo sapiens
ggtcgggttgtgacattgcccgctgtggagat

SEQ ID NO: 84 - DNA, 26 bp, Homo sapiens
ttagggtcataccccatcttggagat

SEQ ID NO: 85 - DNA, 33 bp, Homo sapiens
tgagggtctatgataccacccggtacaggagat

SEQ ID NO: 86 - DNA, 27 bp, Homo sapiens
ttagggtcacacccaccactgggagat

SEQ ID NO: 87 - DNA, 22 bp, Homo sapiens
gaggaggacacccaaggagatc

SEQ ID NO: 88 - DNA, 30 bp, Homo sapiens
tcagggcagtgatgttgcccctcggaagat

SEQ ID NO: 89 - DNA, 34 bp, Homo sapiens
gtggggtagggatattaggccccaattagaagat

SEQ ID NO: 90 - DNA, 33 bp, Xenopus tropicalis
taagggtctgtgacaccaccctctgttggagat

SEQ ID NO: 91 - DNA, 19 bp, Caenorhabditis elegans
tggaatattaccaccggtg

SEQ ID NO: 92 - protein, 209 aa, Homo sapiens
MGSVSNQQFAGGCAKAAEEAPEEAPEDAARAADEPQLLHGAGICKWFNVRMGFGFLSMTARAGVALDPPVDVFVHQSKLHMEGFRSLKEGEAVEFTFKKSAKGLESIRVTGPGGVFCIGSERRPKGKSMQKRRSKGDRCYNCGGLDHHAKECKLPPQPKKCHFCQSISHMVASCPLKAQQGPSAQGKPTYFREEEEEIHSPTLLPEAQN

SEQ ID NO: 93 - protein, 250 aa, Homo sapiens
MAEGGASKGGGEEPGKLPEPAEEESQVLRGTGHCKWFNVRMGFGFISMINREGSPLDIPVDVFVHQSKLFMEGFRSLKEGEPVEFTFKKSSKGLESIRVTGPGGSPCLGSERRPKGKTLQKRKPKGDRCYNCGGLDHHAKECSLPPQPKKCHYCQSIMHMVANCPHKNVAQPPASSQGRQEAESQPCTSTLPREVGGGHGCTSPPFPQEARAEISERSGRSPQEASSTKSSIAPEEQSKKGPSVQKRKKT

SEQ ID NO: 94 - protein, 17 aa, Homo sapiens
RCYNCGGLDHHAKECKL

SEQ ID NO: 95 - protein, 17 aa, Homo sapiens
KCHFCQSISHMVASCPL

SEQ ID NO: 96 - protein, 17 aa, Homo sapiens
RCYNCGGLDHHAKECSL

SEQ ID NO: 97 - protein, 17 aa, Homo sapiens
KCHYCQSIMHMVANCPH

SEQ ID NO: 98 - protein, 72 aa, Homo sapiens
AGICKWFNVRMGFGFLSMTARAGVALDPPVDVFVHQSKLHMEGFRSLKEGEAVEFTFKKSAKGLESIRVTGP

SEQ ID NO: 99 - protein, 72 aa, Homo sapiens
TGHCKWFNVRMGFGFISMINREGSPLDIPVDVFVHQSKLFMEGFRSLKEGEPVEFTFKKSSKGLESIRVTGP

SEQ ID NO: 100 - RNA, 65 nt, Artificial Sequence
  PLK-1 miRNA and Let-7a2 chimeric oligonucleotide
agagacuuaggcacaaucuuuuagaauuacaucaagggagauaaagauugugccuaagucucuuu

SEQ ID NO: 101 - RNA, 49 nt, Artificial Sequence
  PLK-1 miRNA and control loop chimeric oligonucleotide
agagacuuaggcacaaucuuucaagagaagauugugccuaagucucuuu

SEQ ID NO: 102 - RNA, 34 nt, Artificial Sequence
  Consensus sequence
gguagggucurugayayyrcccgsuryrggagau